SEQUENCE LISTING (1 sequence)

SEQ ID NO: 1 - protein, 4 aa, unidentified
NANP

(19) United States
(12) Patent Application Publication
(10) Pub. No.: US 2025/0360086 A1
(43) Pub. Date: Nov. 27, 2025

(54) ENCAPSULATION METHOD AND PARTICLE

(71) Applicants: Oxford University Innovation Limited, Oxford (GB); Coventry University, Coventry (GB)

(72) Inventors: Romain GUYON, Oxford (Oxfordshire) (GB); Anita MILICIC, Oxford (Oxfordshire) (GB); Eleanor STRIDE, Oxford (Oxfordshire) (GB); Adrian HILL, Oxford (Oxfordshire) (GB)

(21) Appl. No.: 18/851,749

(22) PCT Filed: Apr. 6, 2023
(86) PCT No.: PCT/GB2023/050933

§ 371 Date: Sep. 27, 2024

(30) Foreign Application Priority Data
Apr. 7, 2022 (GB) 2205123.9

(51) Int. Cl. A61K 9/50; A61K 39/385

(52) U.S. Cl. CPC A61K 9/5021; A61K 9/5089; A61K 39/385

(57) ABSTRACT

Provided herein is a device for producing an aqueous-core polymeric-shell particle as described herein. Also provided are methods of preparing such particles, as well as the particles themselves. The particles are useful in medicine, particular in the context of vaccines.

### FIELD

The present disclosure relates to the preparation of particles comprising an aqueous core including a pharmaceutical agent encapsulated by a polymer shell.

### BACKGROUND

A cornerstone of pharmaceutical therapy is ensuring that the administered pharmaceutical agent reaches the appropriate part of the body in such a manner to exert its therapeutic effect. Whilst for some therapies, such as single dose vaccinations, immediate release of the active agent into the body is required (e.g. to exert the clinical benefit as soon as possible), this is not always desired. In some cases, delayed or sustained release formulation can be used in addition to or instead of immediate release formulations.

Delayed drug release can be achieved in a variety of means according to the therapeutic application.

Certain agents can be beneficially delivered by controlled release devices such as infusion pumps, which can be programmed to administer therapeutic agents according to a predetermined schedule. Such pumps range from large fixed-position devices to smaller wearable devices. However, they share disadvantages of being physically invasive, expensive, and requiring careful control to ensure the correct dose of medicament.

Other methods have relied upon formulation chemistry to provide medicaments in a form appropriate for controlled release therapy. A wide variety of approaches have been considered.

For pharmaceutical agents preferentially administered systemically (e.g. by intravenous injection), droplets and microencapsulation technologies have been widely proposed. One example relates to the use of liposomes. Liposomes are spherical vesicles having at least one lipid (e.g. phospholipid) layer surrounding an aqueous core. Delivery can be effected by fusing the liposome to a cell membrane, or via diffusion. However, whilst liposomes have shown some promise, problems remain. Ensuring sufficient stability of liposomes can be challenging, with the lipids used to form the liposomes typically susceptible to degradation by oxidation and hydrolysis. Physical damage to the fragile liposome structure can also result in uncontrolled drug release. Furthermore, the reticular-endothelial system typically operates to rapidly clear liposomes from the body once administered. In addition, the cost of the lipids used in their production can be high, and the complexity of forming homogenous samples appropriate for drug delivery requires specialist equipment which further increases costs. Alternative and/or improved methods are therefore required.

One alternative strategy that has been proposed is the formation of droplets wherein the pharmaceutical agent is encapsulated in a polymer. The usual approach adopted in the art involves dissolving the active agent and a polymer in an appropriate solvent and forming a single emulsion in an aqueous phase for delivery. Extraction of the solvent leads to a porous matrix comprising the active agent, which is released over time as a function of (e.g.) the porous nature of the matrix and the degradation of the polymer in vivo. However, whilst such methods typically result in a sustained release profile, the controlled delay release of the agent is much more challenging: in these matrix-based approaches release of the pharmaceutical agent typically occurs immediately following administration with a release profile determined by the gradual breakdown of the matrix over time. These approaches are therefore unsuitable for applications wherein a delayed release profile is required, in which release of the agent occurs only after some time, rather than immediately upon administration.

One context in which delayed (as opposed to sustained) release formulations are required is vaccination. Vaccines have been developed against some of the most prevalent diseases worldwide such as diphtheria, tetanus, pertussis, influenza and measles and contribute to saving millions of lives per annum.

Many vaccines require multiple doses to induce long-lasting and strong protective immunity. For example, multiple vaccine doses may be administered with the timing between doses controlled in order to enhance adaptive immune response. Repeated administrations may be referred to prime/boost regimens, with the first administration “priming” the immune system e.g. by activating naïve T and B cells to proliferate and differentiate into memory cells. A second administration may then “boost” the action of memory cells both by restimulation and also by priming new naïve cells. Prime/boost vaccination regimens include homologous prime/boost strategies in which the same active agent is administered in both the prime and boost stages, and heterologous prime/boost strategies in which different active agents are administered at the prime and boost stages.

Regardless of the exact agents administered, prime/boost vaccination strategies rely on multiple doses of the or each therapeutic agent. This is disadvantageous compared to a single administration for several reasons. Patient compliance may be reduced due to the need to have multiple doses administered. Costs of medical personnel may be elevated due to the need to administered multiple doses. Costs of distributing and storing multiple doses of a vaccine may be higher than handling of a single dose. Furthermore, any deviation from the optimum timeframe between prime and boost administrations (e.g. caused by logistical difficulties in arranging for patients to be at medical facilities at the right time) can result in decreased immunogenicity.

Accordingly, there is a pressing need for new technologies for delivery of vaccines, including delivery of multiple doses of agents such as could be used in a prime/boost vaccination regimen.

The present inventors have sought to develop delayed release drug formulations that can overcome the problems of current delivery systems for vaccines and related therapeutic agents. The inventors have recognised that a formulation method for precisely controlling drug release would have widespread application, including (but not exclusively) in the field of vaccinations. For example, a controlled delayed release formulation technique could be used in conjunction with a conventional formulation of a vaccine, thus providing both prime and boost doses in a single administration. The use of such formulations would allow for reduced costs, simplified storage, would increase patient compliance, and would ensure a precise timeframe between the prime and boost doses. The disclosed devices and methods address some or all of these limitations.

### SUMMARY

According to a first aspect of the present invention, there is provided a method of preparing particles comprising an aqueous core encapsulated by a polymer shell, the method using a flow chip having channels formed therein including a main channel, the method comprising: flowing a solvent phase having droplets of the aqueous phase entrained therein through a transfer section of the main channel, wherein the aqueous phase comprises a pharmaceutical agent and an aqueous solvent, and the solvent phase comprises a polymer and a non-aqueous solvent; flowing an extraction phase through extraction phase inlet channels in the flow chip, the extraction phase comprising an extraction solvent, wherein the extraction phase inlet channels are provided on opposite sides of the main channel, and the transfer section and the extraction phase inlet channels open into an extraction phase intersection section of the main channel, so that droplets of the solvent phase, which encapsulate droplets of the aqueous phase, are formed in the extraction phase; flowing the extraction phase having the droplets of the solvent phase entrained therein through an extraction section of the main channel extending from the extraction phase intersection section; and extracting the non-aqueous solvent of the aqueous phase, so that the particles are formed with the aqueous core being formed by the droplets of the aqueous phase and the shell being formed by the polymer, wherein the main channel has an increase in height at a location where the transfer section opens into the extraction phase intersection section or downstream thereof.

Using this method, it is possible to prepare particles comprising an aqueous core encapsulated by a polymer shell that are robust and homogenous, and have controllable core-shell properties.

As described in more detail herein, the particles made by this method typically comprise a pharmaceutical agent in an aqueous phase comprising an aqueous solvent; surrounded by a homogenous, uniform, solid shell comprising a biocompatible polymer. The particles themselves are typically robust, spherical, and uniform in morphology and structure. As their physiochemical properties can be controlled as described herein, they are ideal for delivery of drugs in many different contexts, including in the context of vaccine delivery. For example, the particles may be used to deliver a discrete “boost” dose of a pharmaceutical agent in a prime/boost vaccination strategy.

Whilst polymeric particles comprising a pharmaceutical agent have been disclosed in the art, the inventors have found that such particles are typically incapable of providing a controlled delayed release of the agent. In particular, whilst many particles are inaccurately depicted in the literature as having a core surrounded by an intact shell, the reality is that such particles typically comprise a complex matrix of the polymer in which the agent is dispersed. Accordingly, as discussed above, release of the agent from the polymer is typically not discrete: rather, gradual uncontrolled dispersion of the active agent from the polymer occurs over time. Furthermore, the particle produced in accordance with previously known methods are typically non-uniform in size and thickness. They are further typically unsuitable for the effective delivery of pharmaceutical agents in practice as the incorporation of the pharmaceutical agent into the polymeric matrix typically leads to degradation of the pharmaceutical agent. For example, the polymeric matrix itself can in some circumstances lead to degradation, for example by reaction of the pharmaceutical agent with functional groups on the polymer. Alternatively or additionally, the exposure of the pharmaceutical agent to harsh conditions (for example, contact with organic solvents and/or low pH solutions) during formation of the matrix and subsequent particle production can often lead to degradation of the pharmaceutical agent, for instance through loss of conformational structure (particularly for protein-based pharmaceutical agents); chemical degradation; and/or aggregation. Furthermore, the efficiency of encapsulation of the pharmaceutical agent (i.e. drug loading) is poor as much of the internal volume of each particle consists of inactive polymer. Quality control is also hampered due to the non-homogeneity of the population. Such prior art particles would not be useful in applications wherein the controlled discrete release of active agent is required, for example in a prime/boost vaccine.

By contrast, the present inventors have found that the disclosed methods produce true core-shell microparticles having an intact shell surrounding an aqueous core. The particles allow the demonstrable controlled, discrete release of the core from the shell and thus are significantly different to those in the art which allow only gradual and/or uncontrolled release of the agent from the matrix. Furthermore, the particles are uniform in size and thickness. They are ideally suited to the effective delivery of pharmaceutical agents without degradation of the agent, in particular because the pharmaceutical active can be isolated from any solvent and/or polymer phase involved in the production and storage of the particles. The efficiency of encapsulation of the pharmaceutical agent (i.e. drug loading) is also high. Quality control is also improved due to the homogeneity of the population.

The advantageous preparation of robust and homogenous particles having controllable core-shell properties is achieved by arranging the main channel to have an increase in height at a location where the transfer section opens into the extraction phase intersection section or downstream thereof. This reduces the compression of the droplets in the main channel with the result of providing stable particles with good loading of the pharmaceutical agent and high levels of mono-dispersity of the aqueous core.

Preferably, the main channel may have an increase in height at a location where the transfer section opens into the extraction phase intersection section. This location for the increase in height has been found to produce particles having particularly advantageous properties.

Alternatively, the main channel may have an increase in height downstream of the location where the transfer section opens into the extraction phase intersection section and upstream of the end of the nozzle section. This location for the increase in height has also been found to produce particles having advantageous properties.

The method may further comprise forming the droplets of the aqueous phase in the solvent phase by: flowing the aqueous phase through an aqueous phase inlet section of the main channel; and flowing a solvent phase through solvent phase inlet channels in the flow chip, wherein the solvent phase inlet channels are provided on opposite sides of the main channel, and the aqueous phase inlet section and the solvent phase inlet channels open into an solvent phase intersection section of the main channel, so that the droplets of the aqueous phase are formed in the solvent phase, the transfer section extending from the solvent phase intersection section.

According to a second aspect of the present invention, there is provided an aqueous-core polymeric-shell particle, comprising:
an aqueous core comprising a pharmaceutical agent; and
a homogeneous solid biodegradable polymeric shell encapsulating the aqueous core.

The aqueous core comprises the pharmaceutical agent in an aqueous phase comprising an aqueous solvent; and is surrounded by a homogenous, uniform, solid shell comprising the biocompatible polymer.

Such particles may be manufactured using the methods disclosed herein.

The particles are typically robust, spherical, and uniform in morphology and structure. As their physiochemical properties can be controlled as described herein, they are ideal for delivery of drugs in many different contexts, including in the context of vaccine delivery. For example, the particles may be used to deliver a discrete “boost” dose of a pharmaceutical agent in a prime/boost vaccination strategy.

The particles can be present in the form of a pharmaceutical composition. The pharmaceutical composition comprises the particle (or a plurality thereof) and one or more pharmaceutically acceptable excipient, diluent or adjuvant. Such components are described in more detail herein.

A disclosed pharmaceutical composition may comprise one or more additional pharmaceutical agents in addition to the particles provided herein. Such a composition may be particularly useful as a vaccine, for example as a prime/boost vaccine composition.

According to a third aspect of the present invention, there is provided a flow chip for preparing particles comprising an aqueous core encapsulated by a polymer shell, the flow chip having channels formed therein which comprise a main channel and extraction phase inlet channels provided on opposite sides of the main channel, wherein the main channel comprises: a transfer section; a extraction phase intersection section, into which the transfer section and the extraction phase inlet channels open; and an extraction section extending from the extraction phase intersection section, wherein the main channel has an increase in height at a location where the transfer section opens into the extraction phase intersection section or downstream thereof.

Such a flow chip may be used in a method according to the first aspect of the present invention.

Advantageously, the flow chip may be further configured so that the channels further comprise solvent phase inlet channels on opposite sides of the main channel, and the main channel further comprises: an aqueous phase inlet section; and a solvent phase intersection section, into which the aqueous phase inlet section and the solvent phase inlet channels open, the transfer section extending from the solvent phase intersection section.

### BRIEF DESCRIPTION OF THE FIGURES

FIG. 1 is a plan view of a flow chip used to prepare particles;

FIGS. 2a and 2b are each an aligned plan view and oblique cross-sectional view of the flow chip, the cross section taken along a main channel, for two alternative constructions;

FIGS. 3a to 3c are each an aligned plan view and oblique cross-sectional side view of the flow chip the cross section taken along a main channel, for three further alternative constructions;

FIG. 4 is an image of the flow chip captured during the preparation of the particles;

FIG. 5 is a set of three images of the downstream part of the flow chip showing the formation of droplets of the aqueous phase are formed in the solvent phase;

FIG. 6 is a set of three time-sequence images of the upstream part of the flow chip showing the formation of droplets of the solvent phase are formed in the extraction phase in a dripping regime;

FIG. 7 is a another set of three time-sequence images of the upstream part of the flow chip showing the formation of droplets of the solvent phase are formed in the extraction phase in a jetting regime;

FIG. 8 is a pair of images of the prepared particles, the left image showing the particles output from the flow chip and the right image showing the particles after washing.

and wherein the signal corresponding to a labelled API (fluorescent dextran) is shown in red in the original colour images. Results are described in Example 1;

FIG. 9 is an image of use of a comparative example of a flow chip without a change in height in the main channel captured during the preparation of the particles;

FIG. 10 is an image of the particles resulting from the preparation shown in FIG. 9, wherein the signal corresponding to a labelled API (fluorescent dextran) is shown in yellow in the original colour images;

FIG. 11 is an image of the flow chip captured during the preparation of the particles showing the effect of the change in height of the main channel at the location shown in FIG. 2a;

FIG. 12 is an image of the particles resulting from the preparation shown in FIG. 11, wherein the signal corresponding to a labelled API (fluorescent dextran) is shown in yellow in the original colour images;

FIG. 13 is a plan view of the flow chip showing regions where the surfaces of the channels hydrophobic and hydrophilic;

FIG. 14 shows the effect of varying polymer molecule weight on the release profile of the particles produced in accordance with the present disclosure. Results are described in Example 2;

FIG. 15 shows the effect of varying the polymeric shell thickness on the release profile of the particles produced in accordance with the present disclosure. Results are described in Example 3;

FIG. 16 shows the effect of varying the L:G ratio on the release profile of the particles produced in accordance with the present disclosure wherein the polymer used is PLGA. This effect is shown as a graph of cumulative release over time. Results are described in Example 4;

FIG. 17 shows the effect of varying the relative injection pressures of the aqueous phase and solvent phase used in the disclosed methods on the morphology of the particles produced in accordance with the present disclosure. Results are described in Example 5;

FIG. 18 shows a comparison of the immunological response in vivo following administration of ovalbumin formulated in intact particles in accordance with the present disclosure, compared to when formulated in crushed particles of the present disclosure, or when formulated together with particles produced in accordance with the present disclosure but without a pharmaceutical agent in the aqueous core. Results are described in Example 6;

FIG. 19 shows a comparison of the IgG response in mice following immunization using R21 formulated either in a soluble form (Ag-Sol) or encapsulated inside particles in accordance with the present disclosure (Ag-Par). Results are described in Example 7;

FIG. 20 shows (A) morphology of heterogeneous particles produced by batch emulsification (vortexing of all phases) and (B) a comparison of the in vitro release profile of particles produced in accordance with the present disclosure (right hand trace; shown in red in the original colour images) against the heterogeneous particles (left hand trace; shown in blue in the original colour images) of (A). Results are described in Example 8.

FIG. 21 shows immune response kinetics of vaccination regiments including encapsulated R21 compared to the unencapsulated vaccine. Antigen-specific IgG titres were measured by ELISA against Cterm-epitopes of the R21 vaccine in BALB/c mice (n=8 per regimen) for each of the four regimens tested. Titres are measured from tail blood sampling every week following immunisation and represented by median and interquartile range. Results described in Example 9.

FIG. 22 shows that peak antibody response of encapsulated non-adjuvanted R21 is non-inferior to the non-encapsulated vaccine. Peak observed specific IgG against Cterm-epitopes are plotted for each mouse (n=8 per regimen). Fold change of the mean peak log titre Cterm-specific responses are estimated by bootstrapping and reported as geometric mean fold change with 95% CI (error bars) and sampling distributions (density plots). Non-inferiority intervals at a 0.5-fold change threshold are displayed as faded rectangles for each comparison.

FIG. 23 shows the effect of the addition of adjuvant on the immune response kinetics of encapsulated R21 regimens compared to unencapsulated controls. Specific IgG are measured by ELISA against Cterm epitopes of the R21 vaccine in BALB/c mice (Prime-Adj n=16, other regimens n=8). Titres are measured from tail blood sampling every week following immunisation and represented by the Median and interquartile range.

FIG. 24 shows peak antibody response of encapsulated R21 with adjuvant is noninferior to the unencapsulated control. Peak observed titres of specific IgG against Cterm-epitopes are shown for each mouse (Prime-Adj n=16, other regimens n=8). Fold change of the mean peak log titre for Cterm-specific responses are estimated by bootstrapping and reported as geometric mean fold change with 95% CI (error bars) and sampling distributions (density plots). Non-inferiority intervals at a 0.5-fold change threshold are displayed as shaded rectangles for each comparison.

FIG. 25 shows longitudinal Cterm-specific immune kinetic profiles for different LCSS particle formulations. Cterm-specific titres are measured by ELISA for adjuvanted R21, encapsulated in different LCSS particle formulations or nonencapsulated (prime only n=16, other regimens n=8). Titres are measured weekly following immunisation, and data for individual mice are joined by grey lines. Group-level responses from generalised additive model (GAM) fits are displayed for each regimen as coloured lines in original colour image, with the 95% CI represented by ribbons.

FIG. 26 shows estimates of the peak titre and time to 50% peak titre. Group-level responses from generalised additive model (GAM) fits for Cterm-specific IgG responses are displayed for each regimen as coloured lines in original colour image, with the 95% CI represented by ribbons. Vertical lines indicate the time to 50% of peak titre. Geometric mean peak antibody titre and mean time to 50% peak titre of the 10,000 simulated samples (faded dots for each simulated samples) with 95% confidence regions (shaded areas) and 95% marginal CI (error bars).

FIG. 27 shows fold change of peak antibody titres and additional immune response delay for different LCSS particle formulations compared to unencapsulated control. Geometric mean fold change of Cterm-specific peak antibody titre and mean additional delay in time to reach 50% Cterm-specific peak titre for different LCSS particle formulations compared to unencapsulated control. Error bars represent 95% CIs, density plots represent the simulated sampling distributions. Non-inferiority intervals at a 0.5-fold change threshold are displayed as shaded rectangles.

FIG. 28 shows design summary of the in vivo immunogenicity and malaria challenge experiment comparing the efficacy of the Prime+Particles regimen with Prime only. BALB/c mice were immunised IM as illustrated. Injections were performed using 100 μL Hamilton glass syringes sterilised with ethanol and 25G needles. Injection volume was 50 μL with 0.5% carboxymethylcellulose in PBS as a viscoenhancing agent to prevent LCSS particle aggregation in the syringe. Challenge was performed in two challenges sessions, by IV injection (into the tail vein) of 1000 P. falciparum sporozoites per mouse. Peak time windows displayed here correspond to the 95% CI of time to 50% of peak titre in Cterm-specific immunogenicity.

FIG. 29 shows immune response kinetics of different R21/adjuvant regimens in the Short study challenge. Specific IgG are measured by ELISA against Cterm epitopes of the R21 vaccine in BALB/c mice (Prime/Boost regimen n=8, other regimens n=16) for each of the three regimens tested. Titres are measured from tail blood sampling every week following immunisation until the challenge session and represented by the Median and interquartile range. Booster timing for the Prime/Boost regimen is represented by a vertical dashed line.

FIG. 30 shows peak antibody Cterm-specific response in the Prime and Short PAR regimen is superior to nonencapsulated Prime and Prime/Boost regimens. Peak observed titres of specific IgG against Cterm epitopes are shown for each mouse (all regimens n=8). Fold change of the mean peak log titre for Cterm-specific responses are estimated by bootstrapping and reported as geometric mean fold change with 95% CI (error bars) and sampling distributions (density plots).

FIG. 31 shows immune response kinetics of different R21/adjuvant regimens in the Short study challenge. Specific IgG are measured by ELISA against Cterm epitopes of the R21 vaccine in BALB/c mice (Prime/Boost regimen n=8, other regimens n=16) for each of the three regimens tested. Titres are measured from tail blood sampling every week following immunisation until the challenge session and represented by the Median and interquartile range. Booster timing for the Prime/Boost regimen is represented by a vertical dashed line.

FIG. 32 shows peak antibody Cterm-specific response of Prime and Medium PAR regimen is superior to nonencapsulated Prime regimen. Peak observed titres of specific IgG against Cterm epitopes are shown for each mouse (Prime/Boost regimen n=8, other regimens n=16). Fold change of the mean peak log titre for Cterm-specific responses are estimated by bootstrapping and reported as geometric mean fold change with 95% CI (error bars) and sampling distributions (density plots).

FIG. 33 shows that Prime+Short PAR is more protective and delays time to malaria infection compared to Prime only. BALB/c mice were immunized with different regimens (n=8 for Prime/Boost, n=16 for others) and challenged by IV injection of 1000 sporozoites. Prime/Boost mice were challenged 2 weeks after the booster (4 weeks after the first) immunisation, whereas other regimens were challenged at either 3 or 4 weeks after immunisation in two challenge sessions, n=8 per regimen at each challenge session. Blood stage parasitaemia was monitored for a week starting from day 5 after challenge. Kaplan Meier curves for infection (1% parasitaemia) are plotted for each regimen, pooling data from the two challenge sessions. The table shows the hazard ratios (estimated by Cox regression) for each regimen compared to Prime. Hazard ratios are displayed with their respective 95% CI and p values.

FIG. 34 shows that Prime+Medium PAR is more effective at delaying time to malaria infection compared to Prime only. BALB/c mice were immunized with different regimens (n=8 for Prime/Boost, n=16 for others) and challenged by IV injection of 1000 sporozoites. Mice receiving the Prime/Boost regimen were challenged 2 weeks after the boost (5 weeks following the first immunisation) whereas other regimens were challenged at either 5 or 6 weeks after immunisation in two challenge sessions, n=8 per regimen at each challenge session. Blood stage parasitaemia was monitored from day 5 following challenge. Kaplan Meier curves for infection (1% parasitaemia) are plotted for each regimen pooling challenge sessions. The table shows the hazard ratios (estimated by Cox regression) for each regimen compared to Prime. Hazard ratios are displayed with their respective 95% CI and p values.

### DETAILED DESCRIPTION

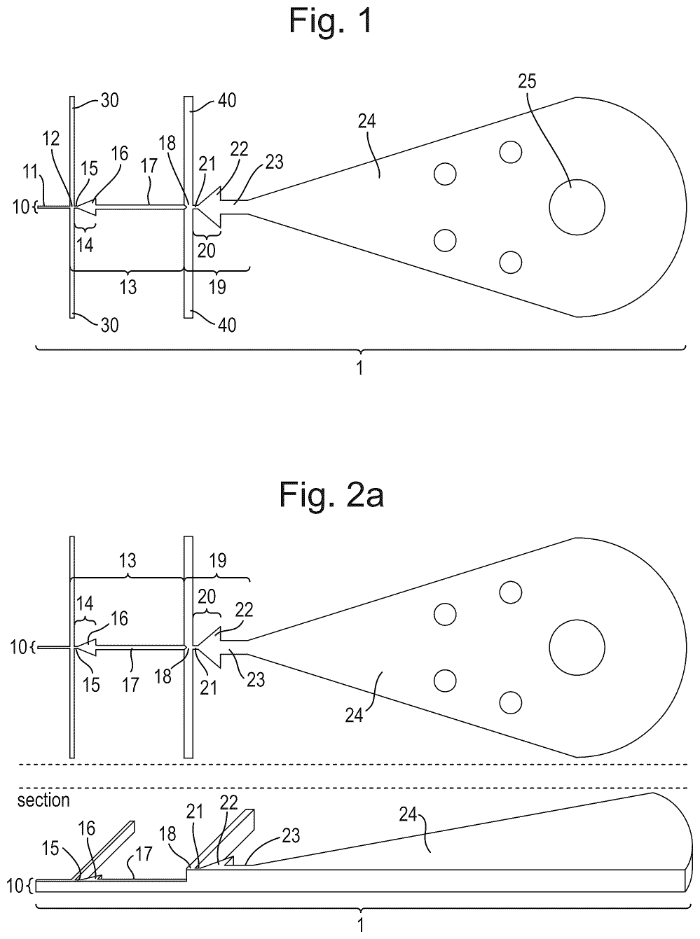

FIG. 1 shows a flow chip 1 for preparing particles comprising an aqueous core encapsulated by a polymer shell. The flow chip 1 is a body of material in which channels are formed. The arrangement of the channels from the upstream end to the downstream end is as follows. The channels have planar extent. The width of the channels is defined in a direction of the planar extent and the height of the channels is defined in a direction orthogonal to the planar extent.

The flow chip 1 has a main channel 10 in which the particles are formed. As described below, flow various liquids are flowed through the main channel 10, the upstream end being left and the downstream end being right in FIG. 1.

The main channel 10 comprises an aqueous phase inlet section 11 at the upstream end. In use, an aqueous phase is flowed into the flow chip 1 through the aqueous phase inlet section 11. As discussed further below, the aqueous phase comprises a pharmaceutical agent and an aqueous solvent.

The flow chip 1 has solvent phase inlet channels 30 on opposite sides of the main channel 1. In use, a solvent phase is flowed into the flow chip 1 through solvent phase inlet channels 30. As discussed further below, the solvent phase comprises a polymer and a non-aqueous solvent.

The main channel 10 comprises a solvent phase intersection section 12, into which the aqueous phase inlet section 11 and the solvent phase inlet channels 30 open. The solvent phase inlet channels 30 extend at 90° to aqueous phase inlet section 11 so that the solvent phase intersection section 12 provides an intersection that is a 90° cross-junction. In use, the aqueous phase and the solvent phase flowing into the solvent phase intersection section 12 form droplets of the aqueous phase in the solvent phase.

The main channel 10 comprises a transfer section 13 extending from the solvent phase intersection section 12.

The transfer section 13 comprises a solvent nozzle section 14 adjacent to, and downstream of, the solvent phase intersection section 12. The solvent nozzle section 14 has an increase in width with distance from the solvent phase intersection section 12. In particular, the solvent nozzle section 14 comprises a neck section 15 having constant width, and an expansion section 16 downstream of the neck section and having an increase in width with distance from the solvent phase intersection section 12. In use, the solvent nozzle section 14 assists in the formation of the droplets of the aqueous phase in the solvent phase.

The transfer section 13 comprises a flow section 17 downstream of the solvent nozzle section 14, the flow section 17 having a lower width than the solvent nozzle section 14, providing a constriction in the main channel 10.

The flow chip 1 has extraction phase inlet channels 40 provided on opposite sides of the main channel 10. In use, an extraction phase is flowed into the flow chip 1 through the extraction phase inlet channels 40. As discussed further below, the extraction phase comprises an extraction solvent for extracting the non-aqueous solvent from the solvent phase.

The main channel 10 comprises an extraction phase intersection section 18, into which the transfer section 13 and the extraction phase inlet channels 40 open. The extraction phase inlet channels 40 extend at 90° to transfer section 13 so that the extraction phase intersection section 18 provides an intersection that is a 90° cross-junction. In use, the solvent phase and the extraction phase flowing into the solvent phase intersection section 18 form droplets of the solvent phase, which encapsulate droplets of the aqueous phase, in the extraction phase.

The main channel 10 comprises an extraction section 19 extending from the extraction phase intersection section 18.

The extraction section 19 comprises an extraction nozzle section 20 adjacent to, and downstream of, the extraction phase intersection section 18. The extraction nozzle section 20 has an increase in width with distance from the extraction phase intersection section 18.

In particular, the extraction nozzle section 20 comprises a neck section 21 having constant width, and an expansion section 22 downstream of the neck section 21 and having an increase in width with distance from the extraction phase intersection section 18. In use, the extraction nozzle section 20 assists in the formation of the droplets of the solvent phase in the extraction phase.

The extraction section 19 comprises a flow section 23 downstream of the extraction nozzle section 20, the flow section 23 having a lower width than the extraction nozzle section 20, providing a constriction in the main channel 10.

In use, the non-aqueous solvent of the aqueous phase is extracted in the extraction section 19 and subsequently, so that the particles are formed with the aqueous core being formed by the droplets of the aqueous phase and the shell being formed by the polymer

The main channel 10 comprises an output section 24 downstream of the extraction section 19 having a higher width than the extraction section 19. In use, the particles are collected in the output section 24. The output section 24 has an outlet 25 through wich extracting the extraction phase and particles may be extracted from the flow chip 1.

The main channel 10 has an increase in height that assists the preparation of particles, as will now be described.

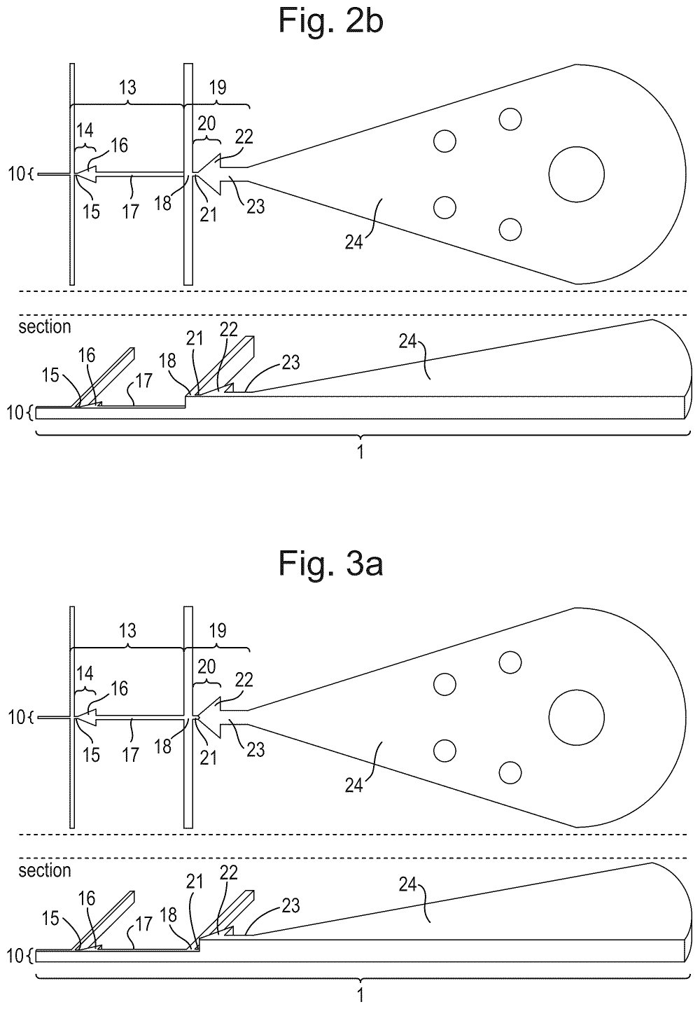

FIGS. 2a and 2b show two alternative constructions in which the main channel 19 has an increase in height at the location where the transfer section 13 opens into the extraction phase intersection section 18. In the alternative construction of FIG. 2a, the increase in height occurs along a line protruding downstream, i.e. into the extraction phase intersection section 18. In particular, the line is formed by two straight line sections meeting at a point (i.e. a V-shape), although the line could alternatively be curved. In the alternative construction of FIG. 2b, the increase in height occurs along a straight line.

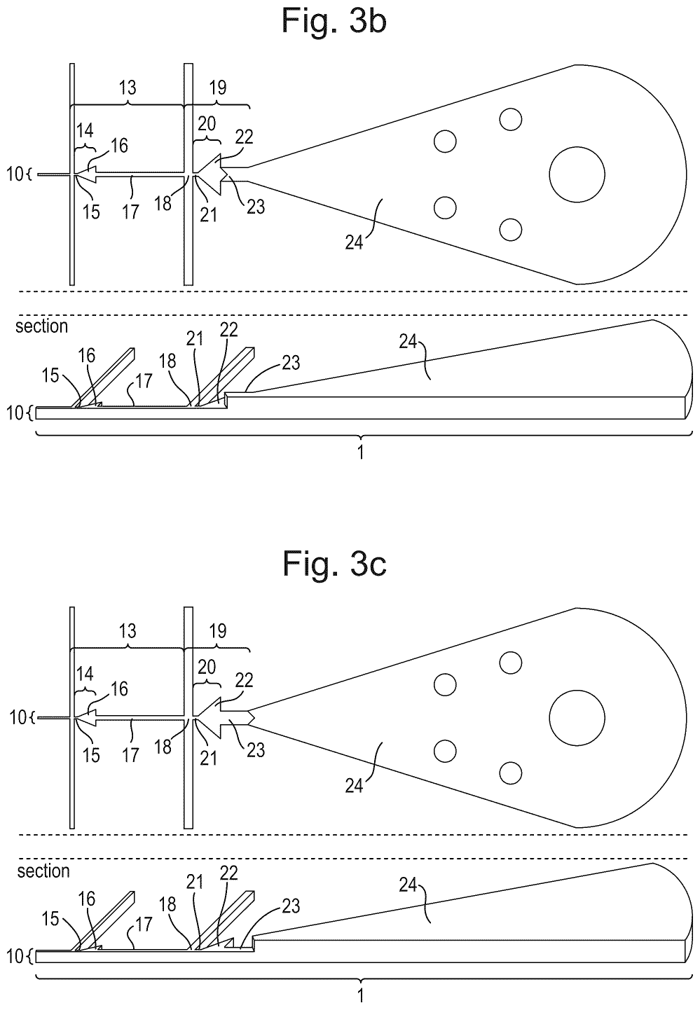

FIGS. 3a to 3c show a second alternative that the main channel 19 has an increase in height downstream of the location where the transfer section 13 opens into the extraction phase intersection section 18.

In the alternative construction of FIG. 3a, the increase in height occurs at the location the neck section 21 of the extraction nozzle section 20 meets the expansion section 22 of the extraction nozzle section 20.

In the alternative construction of FIG. 3b, the increase in height occurs at the location where the expansion section 22 of the extraction nozzle section 20 meets the flow section 23 of the transfer section 19.

In the alternative construction of FIG. 3c, the increase in height occurs at the location where the flow section 23 of the transfer section 19 meets the output section 24.

In each of the alternative construction of FIGS. 3a to 3c, the increase in height occurs along a line protruding downstream, i.e. into the extraction phase intersection section 18. In each case, the line is formed by two straight line sections meeting at a point (i.e. a V-shape)), although the line could alternatively be curved. In each of the alternative construction of FIGS. 3a to 3c, the increase in height could alternatively occur along a straight line.

In FIGS. 2a, 2b and 3a to 3c, the change in height of the main channel 10 is achieved by a step in one of the surfaces of the main channel that are opposed in the height direction. However, the change in height of the main channel 10 may alternatively be achieved by a step in each of the surfaces of the main channel that are opposed in the height direction.

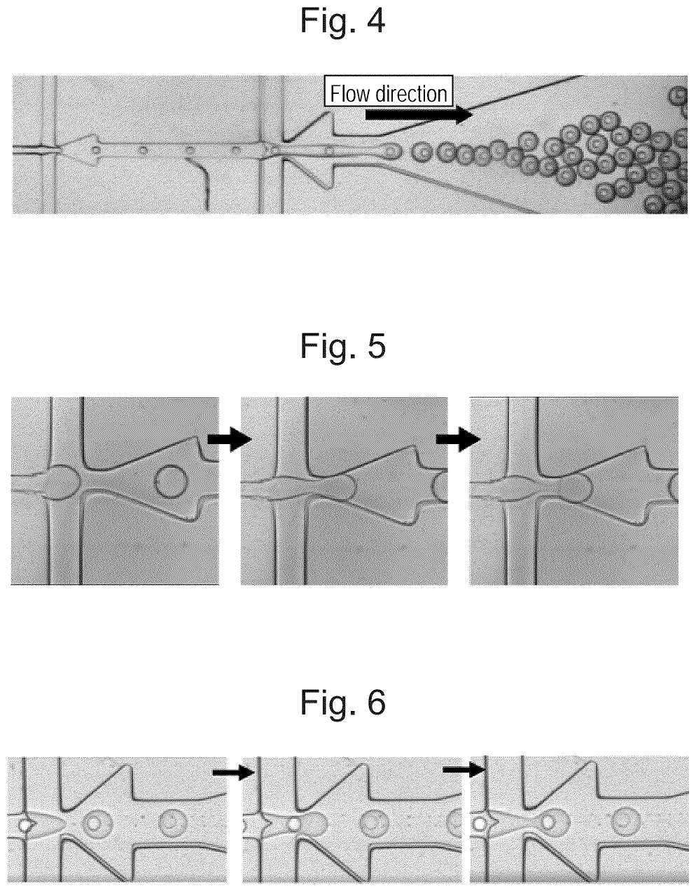

The method using the flow chip to prepare the particles will now be described, with reference to FIG. 4 which shows the formation of particles in the flow chip 1 having a cross-section as shown in FIG. 2a.

The aqueous phase is flowed through an aqueous phase inlet section 11 of the main channel 10.

The solvent phase is flowed through solvent phase inlet channels 30 and meets the aqueous phase in the solvent phase intersection section 12 of the main channel 10. The droplets of the aqueous phase are formed in the solvent phase as shown in FIG. 5.

When the aqueous phase meets the solvent phase in the solvent phase intersection section 12, the two phases are immiscible (completely or partly) such that mixing between the phases does not substantially occur. The solvent phase exerts a shear strain on the aqueous phase, the shear strain being balanced on each side of the aqueous phase, because the solvent phase inlet channels 30 are provided on opposite sides of the main channel 10.

This provides the formation of droplets once the interfacial tension is overcome. Droplets of the aqueous phase may be formed in a dripping manner, creating an emulsion having high mono-dispersity of aqueous droplets inside the solvent phase. The droplets flow in an equally spaced manner along the transfer section 13.

The solvent nozzle section 14 assists in the formation of the droplets of the aqueous phase in the solvent phase as follows. The solvent nozzle section 14 provides a flow focusing geometry after the solvent phase intersection section 12. In particular, the increasing width in the solvent nozzle section 14, in particular in the expansion section 16 of the solvent nozzle section 14, creates a velocity gradient, with the highest velocity being focused at the neck section 15 of solvent nozzle section 14. This velocity gradient increases the shear strain exerted on the aqueous phase by the solvent phase at this point, and causes the aqueous phase to break up into droplets in a repeatable manner. This enables a tight distribution of size of the droplets.

The extraction phase is flowed through the extraction phase inlet channels 40 in the flow chip 1.

The solvent phase having the droplets of the aqueous phase entrained therein are flowed through the transfer section 13 of the main channel 10 and meet the extraction phase in the extraction phase intersection section 18 of the main channel 10. The droplets of the solvent phase, which encapsulate droplets of the aqueous phase, are formed in the extraction phase as shown in FIG. 6.

When the solvent phase meets the extraction phase in the extraction phase intersection section 18, the two phases are immiscible (completely or partly) such that mixing between the phases does not substantially occur. The extraction phase exerts a shear strain on the solvent phase, the shear strain being balanced on each side of the solvent phase, because the extraction phase inlet channels 40 are provided on opposite sides of the main channel 10. This provides the formation of droplets of solvent phase once the interfacial tension is overcome. Droplets of the solvent phase, which themselves encapsulate droplets of the aqueous phase, may be formed in a dripping manner or a jetting manner as described further below. In a general sense, the formation process relies on the shear forces in a similar manner to the process of forming the droplets of aqueous phase in the solvent phase, but the droplets of the solvent phase are formed encapsulating droplets of the aqueous phase, thereby creating a double emulsion. The droplets flow in an equally spaced manner along the extraction section 19.

The extraction nozzle section 20 assists in the formation of the droplets of the solvent phase in the extraction phase, in a similar manner to the effect of the solvent nozzle section 14, as follows. The extraction nozzle section 20 provides a flow focusing geometry after the extraction phase intersection section 18. In particular, the increasing width in the extraction nozzle section 20, in particular in the expansion section 22 of the extraction nozzle section 20, creates a velocity gradient, with the highest velocity being focused at the neck section 21 of the extraction nozzle section 20. This velocity gradient increases the shear strain exerted on the solvent phase by the extraction phase at this point, and causes the solvent phase to break up into droplets in a repeatable manner. This allows the droplets of the solvent phase encapsulating droplets of the aqueous phase to be reliably formed.

The manner of formation of the droplets of solvent phase depends on the flow rate of the solvent phase as follows.

In one case, the droplets of the solvent phase may be formed in a dripping manner immediately after passing through extraction nozzle section 20 as shown in FIG. 6.

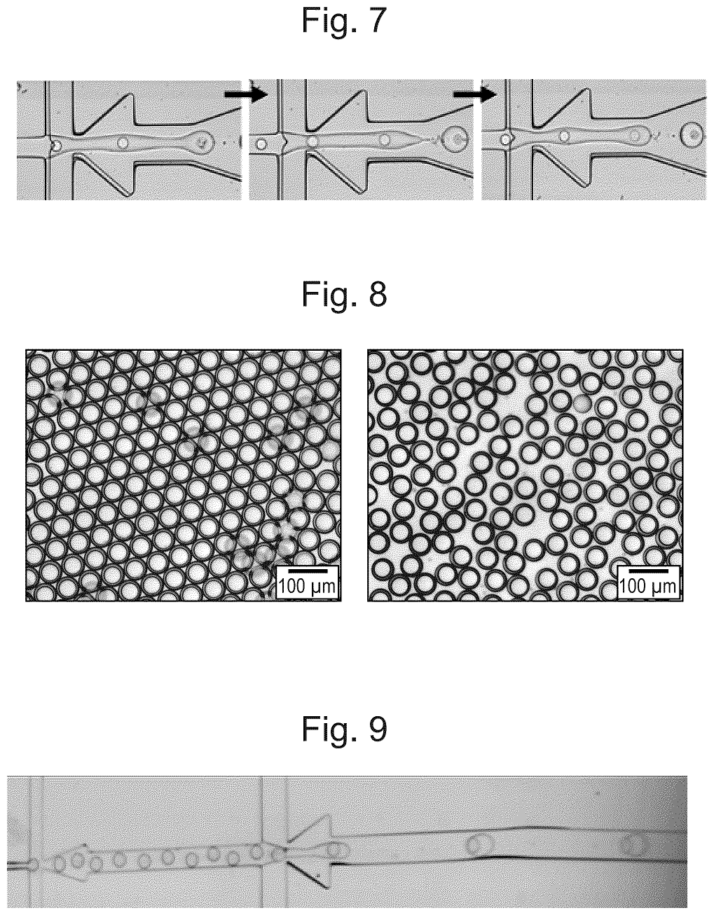

Alternatively, in another case, the solvent phase may produce a longer thread that can ultimately form droplets by jetting due to Rayleigh-Plateau instabilities causing breakup of the liquid thread as shown FIG. 7. In this case, the droplets may form in the flow section 23 downstream of the extraction nozzle section 20, where the constriction accelerates the flow. To summarise the droplet formation mechanism, a perturbation in the thread of solvent phase induced by the passing of an inner droplet of aqueous phase creates sinusoidal instabilities at the interface between the flow of the solvent phase and the outer flow of the extraction phase. Due to the surface tension between the two phases, it results in the break of the thread of the solvent phase into droplets around respective droplets of the aqueous phase.

To enable application of the particles in drug delivery, both a thick outer shell and a high production rate are desirable. These aims may be achieved by use of high flow rates and a solvent phase having a relatively high viscosity, with the highest concentration of dissolved polymer compatible with the microfluidic process. Hence, the extraction phase intersection section 18 is advantageously used in the jetting regime exemplified in FIG. 7, as this has a better throughput for viscous flows.

In these conditions, the solvent phase and entrained droplet of aqueous are confined into a jet that passes through the extraction nozzle section 20, are slowed down in the extraction nozzle section 20, then accelerated again in the following constriction at the flow section 23. This, combined with the passing of the droplet of aqueous phase within this jet of the solvent phase and successions of constriction-expansion-constriction channels, leads to the creation of an instability on the solvent jet, that breaks to form droplets of the solvent phase around each inner droplet of the aqueous phase. As the formation of a droplet of solvent phase is triggered by the passing inner droplet of aqueous phase, this enhances the encapsulation rate and throughput of the production. This is preferable to letting the solvent phase drip and synchronising this dripping with the passing of an inner droplet of aqueous phase, although that is also possible.

The change in height of the main channel 10 also has an effect, but this is described below.

The droplets of the solvent phase are flowed through the extraction section 19 into the output section 24. Thereafter the droplets may be collected from the output section 24 into another container.

The non-aqueous solvent of the aqueous phase is extracted from the droplets of the solvent phase, so that the particles are formed with the aqueous core being formed by the droplets of the aqueous phase and the shell being formed by the polymer. The extraction may occur by liquid phase extraction into the extraction phase, and/or by evaporation.

The extraction starts to occur in the extraction section 19. Depending on the length of the extraction section, the extraction may occur completely in the extraction section 19. Alternatively, the extraction may continue while the droplets are in the output section 24, and/or subsequently after the droplets have been collected from the flow chip 1, for example in an open container such as a petri dish.

In one example, the droplets may be left in the extraction phase in an open container to carry out the extraction of the solvent by evaporation, and thereafter washed to perform a liquid extraction, especially where the solvent used is miscible in water (for example in the case of DMC which is miscible at 13% with water. After a predetermined time, being 48 hrs in this example, chosen to ensure all droplets are converted to particles with a solid shell, the particles can be collected.

Any suitable collection method can be used. For example, the particles can be collected by centrifugation. The particles may be collected by filtration. The particles may be collected by evaporation of the liquid medium in which they are initially prepared, e.g. by evaporation of the extraction phase. Those skilled in the art will appreciate that the particles can be prepared and isolated on a commercially relevant scale. In some embodiments at least 10¹, 10², 10³, 10⁴, 10⁵, 10⁶, 10⁷, 10⁸ or 10⁹ particles are prepared in accordance with the disclosed methods. The collected particles may be optionally washed and/or redispersed, for example using an aqueous carrier solution as defined herein, such as water.

The particles can be used after several washes, by centrifugation and change of supernatant. FIG. 8 shows particles which were collected in this manner, the left-hand side showing the particles in the open container, and the right hand side showing the particles after centrifugation washes.

The particles may be formulated into a pharmaceutical composition comprising said particles and one or more pharmaceutically acceptable excipient, diluent, or adjuvant.

The change in height of the main channel 10 improves the formation of the droplets as follows.

By way of comparison, FIG. 9 shows a comparative example of use of a flow chip 1 in which there is no change in height of the main channel 10. In this case, there is compression of the droplets of the solvent phase in the extraction phase between the surfaces of the main channel 10 that are opposed in the height direction. As a result and as shown in FIG. 9, the flow along the extraction section 19 the flow is deformed, the surfaces are wetted with the potential for later clogging or production disturbance. FIG. 9 also shows trailing of the core towards the tail of the droplet of the solvent phase, resulting in unloading of the particles.

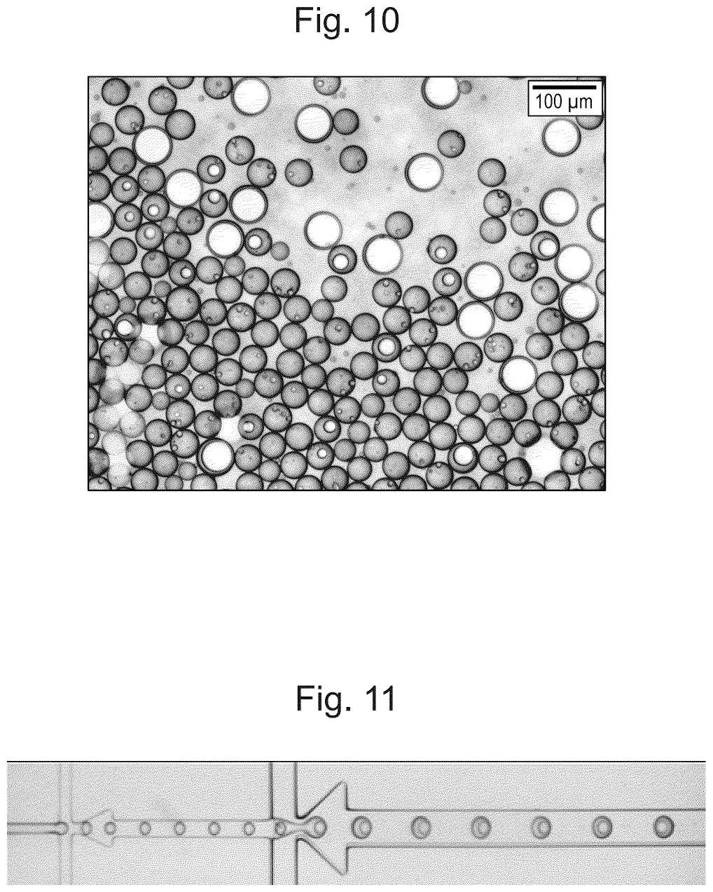

An example of the particles prepared in this comparative example are shown in FIG. 10. As can be seen, the particles form an unloaded and irregular double emulsion. There are many empty polymeric drops, so the content of the pharmaceutical agent is reduced.

In contrast, FIG. 9 shows an example of use of a flow chip 1 in which there is no change in height of the main channel 10 at the location shown in FIG. 2a where the transfer section 13 opens into the extraction phase intersection section 18. The effect of the change in height of the main channel 10 is to remove the compression occurring in the comparative example. This prevents dragging of the droplet of aqueous solution towards the tail of the outer droplet of the solvent phase. It also limits wetting of the transfer section, as can be seen in FIG. 11. Thus, the double emulsions remain stable, and can be collected without losing their core content of the pharmaceutical agent.

In addition, the fact that a double emulsion can be formed in the jetting regime seems also due to the succession of constrictions and expansions that will trigger the double emulsion formation when a droplet passes through this geometry.

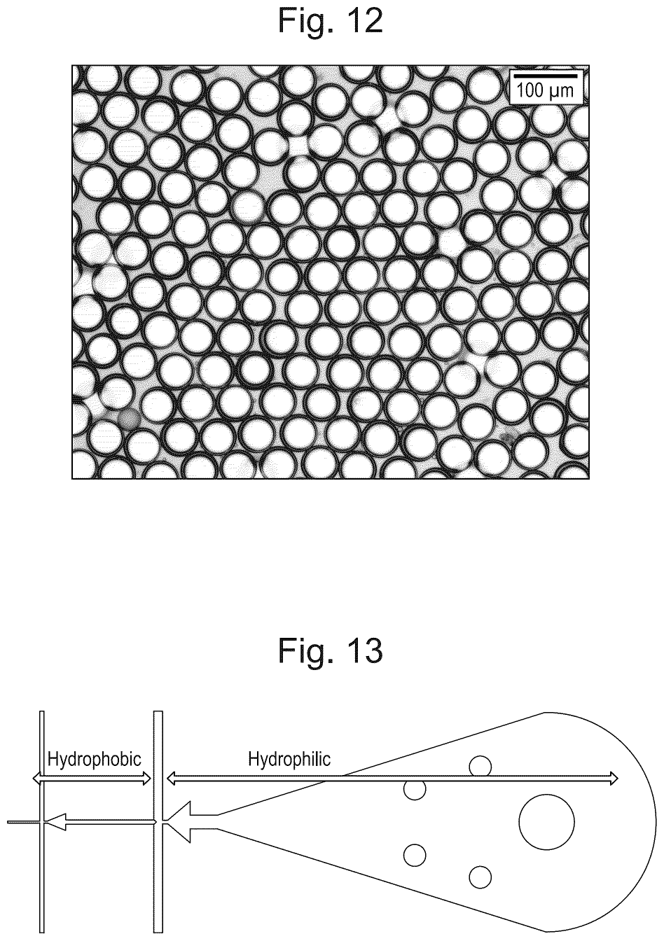

An example of the particles prepared in the example of FIG. 11 is shown in FIG. 12. As can be seen, the particles have good loading and mono-dispersity. Since the solvent phase comprises an extractible solvent to enable formation of solid shells, the potential for clogging which occurs when the extraction starts in the flow chip 1 is reduced. By preserving the particles of the solvent phase from touching the surfaces of the extraction section 19, and giving more space for the droplets of solvent phase to flow in the extraction phase, the risks of disturbing the production process are reduced.

Locating the change in height of the main channel 10 downstream of the location where the transfer section 13 opens into the extraction phase intersection section 18, as in the flow chip 1 shown in FIGS. 3a to 3c, has also been tested and the results show that it produces a similar effect of preserving the newly formed double emulsion.

However, locating the change in height of the main channel 10 at the location where the transfer section 13 opens into the extraction phase intersection section 18 helps to slow down the flow of the solvent phase, which facilitates formation of the particles of solvent phase when the inertial forces are too high (i.e. flows are too high). Thus, for some regimes, this is the preferable design.

The dimensions of the flow chip 1 shown in FIG. 1 are given as follows, together with non-limitative preferred ranges. In particular, the dimensions are such that the channels in the flow chip are not capillary, i.e. the flow chip 1 is not a capillary co-flow microfluidic device. In all the following ranges, the upper and lower limits may be applied independently.
Aqueous phase inlet section 11:
   Width: 20 μm (preferably in a range from 5 μm to 200 μm)
   Height: 50 μm (preferably in a range from 5 μm to 250 μm)
Solvent phase inlet channels 30:
   Width: 40 μm (preferably in a range from 5 μm to 500 μm)
   Height: 50 μm (preferably in a range from 5 μm to 250 μm)
Neck section 15 of solvent nozzle section 14:
   Width: 15 μm (preferably in a range from 5 μm to 200 μm)
   Length: 30 μm (preferably in a range from 10 μm to 200 μm)
Expansion section 16 of solvent nozzle section 14:
   Length: 170 μm (preferably in a range from 100 μm to 500 μm)
   Width: 150 μm (preferably in a range from 100 μm to 500 μm)
   Angle (with respect to axis of main channel 10): 22° (preferably in a range from 10° to 90°)
Flow section 17 of transfer section 13:
   Width: 40 μm (preferably in a range from 5 μm to 500 μm)
Transfer section 17:
   Length: 1 mm (preferably in a range from 0.1 mm to 5 mm)
Main channel 10 downstream of the change in height:
   Height: 50 μm (preferably in a range from 5 μm to 250 μm)
Main channel 10 upstream of the change in height:
   Height: 100 μm (preferably in a range from 20 μm to 500 μm)
Change in height of main channel:
   50 μm (preferably in a range from 15 μm to 250 μm)
Line along which change in height of main channel occurs, in the case where it protrudes downstream:
   Extent along the main channel 10: 25 μm (preferably in a range from 0 μm to 100 μm)
Extraction phase inlet channels 40:
   Width: 80 μm (preferably in a range from 20 μm to 500 μm)
   Height is the same as the main channel 10, so:
      100 μm (preferably in a range from 20 μm to 500 μm) when the change in height of the main channel 10 is at the location where the transfer section 13 opens into the extraction phase intersection section 18
      50 μm (preferably in a range from 5 μm to 250 μm) when the change in height of the main channel 10 is downstream of the location where the transfer section 13 opens into the extraction phase intersection section 18
Neck section 21 of extraction nozzle section 20:
   Width: 30 μm (preferably in a range from 10 μm to 200 μm)
   Length: 50 μm (preferably in a range from 10 μm to 200 μm)
Expansion section 22 of extraction nozzle section 20:
   Length: 200 μm (preferably in a range from 100 μm to 1000 μm)
   Width: 380 μm (preferably in a range from 100 μm to 1000 μm)
   Angle (with respect to axis of main channel 10): 41° (preferably in a range from 10° to 90°)
Flow section 23 of extraction section 19:
   Width: 120 μm (preferably in a range from 10 μm to 200 μm)
   Length: 250 μm (preferably in a range from 100 μm to 1000 μm)
Output section 24:
   Length: 4 mm (preferably in a range from 1 mm to 10 mm)
   Maximum width: 2 mm (preferably in a range from 1 mm to 10 mm)
   Angle (with respect to axis of main channel 10): 17° (preferably in a range from 5° to 90°)

The aqueous phase, solvent phase and extraction phase may be input to the flow chip 1 using a pressure-based system that enables the fluids to be injected at a constant pressure from respective reservoirs, or by using a flow-based system that enables to be injected at a constant flow rate, for example syringe pumps.

Flow rates may be measured by taking optical measurements from within the flow chip 1, measured with before input of the phases into the flow chip 1 with flow sensors, such as thermal or mass flow sensors, placed between reservoirs for the various phases and the flow chip 1, or calculated from the pressure and dimensions of the channels in the flow chip 1.

To maximise production rates, the highest flow rates possible need to be used while maintaining the ability to generate droplets at the solvent phase intersection section 12 and the extraction phase intersection section 18. Desirably to support the microfluidic emulsification process, the flows are laminar (using a relatively low Reynold number Re, for example less than 2000) and dripping and jetting emulsification process are possible (using a relatively low Capillary number (Ca), and Weber number (We). This may be selected by controlling the combination of flow viscosities, interfacial tensions, and flow rates.

Criteria applied to select the flow rates in dependence on the size of cores of aqueous solution and the outer shell obtained after the production are as follows.

Increasing the ratio of the flow rate of the aqueous phase to the flow rate of the solvent phase increases the size of the droplet of the aqueous phase and hence the resultant core of the aqueous phase.

Increasing the ratio of the flow rate of the solvent phase to the flow rate of the extraction phase increases the size of the droplet of the solvent phase and hence the shell of polymer.

Increasing the ratio of the flow rate of the aqueous phase to the flow rate of the extraction phase increases the probability of encapsulating two or more droplets of the aqueous phase inside one droplet of the solvent phase, which is undesirable.

The flow rate is set above a minimum level for the fluids to flow through the flow chip 1 and the continuous phases at each intersection to exert a sufficient shear stress to form emulsions.

The flow rate is set above a maximum level that creates a continuous and stable jet of the phases without emulsification. The threshold estimation depends on the properties of the fluids that changes depending on the particle formulation (different API, API buffer, polymer, polymer molecular weight, etc. . . . ), but may be experimentally derived for any combination of chemistries.

Some non-limitative but preferred flow rates for the flow chip shown in FIG. 1 are as follows:
Flow rate of aqueous phase: 0.25 nL/s to 30 μL/s
Flow rate of solvent phase: 2.5 nL/s to 150 μL/s
Flow rate of extraction phase: 12 nL/s to 3 mL/s

The flow chip 1 may be manufactured by any suitable process.

A preferred option is to manufacture the flow chip 1 by multilayer moulding. Such a process may use moulds produced by multi-layer soft lithography (for example available from Micrux microfluidix, Spain).

The multi-layer soft lithography process is known and similar to the single layer process. Briefly, as for the single layer soft lithography, a layer of negative photoresist (SU-8) is spin coated on a silicon wafer. It is then selectively exposed to UV with a mask containing the features with the first height. Then another layer of negative photoresist is spin coated and exposed to UV through another mask containing only the designs with the extended height. The mould is developed to form the corresponding pattern as a positive mould. This positive mould is treated with trimethylchlorosilane to prevent the sticking of the material of the flow chip 1 (for example PDMS). Then, the uncured material is poured onto the mask and cured, to engrave the channels in a solid block of the material of the flow chip 1 (negative pattern). Inlets and Outlets are punched in the flow chip 1 and the design is sealed by bonding the block with engraved channels to a bottom layer of the material of the flow chip 1, for example using O₂ plasma cleaner.

The flow chip may be made from any suitable material. Typically the flow chip comprises a body comprising or consisting of a mouldable material. Exemplary materials include polymers such as PDMS (polydimethylsiloxane), PMMA (polymethylmethacrylate), polycarbonate (PC), or cyclic olefin copolymer (COC); and glass. When the flow chip comprises a body comprising or consisting of glass the glass may be treated e.g. in order to control the chemistry of the solvent-accessible surface. For example, glass surfaces may be treated with silanes in order to control the hydrophobicity of the surface. Silane modification of surfaces such as glass is well known in the art and silane reagents are commercially available, e.g. from Gelest. Hydrophobic silanes include methyl silanes, linear or branched alkyl silanes, aromatic silanes, dialkyl silanes, etc. Fluorinated silanes may be used to further increase hydrophobicity. Hydrophilicity can be increased by modifying a glass surface with a silane reagent comprising one or more functionalised hydrocarbon groups, being functionalised with hydrophilic functional groups. For example, hydroxy or amino functionalised alkanes can be used. Polymeric surfaces can be modified similarly. For example, PDMS is inherently hydrophobic, but can be treated with hydrophilic polymers e.g. with polyvinyl alcohol in order to increase its hydrophilicity. For example, a solution of 1% of polyvinyl alcohol (PVA) in water may be introduced into the channels of a chip after its production to increase the hydrophilicity of the selected areas (other areas may be protected by injection of air); e.g. see Trantidou, T., Elani, Y., Parsons, E. et al. Hydrophilic surface modification of PDMS for droplet microfluidics using a simple, quick, and robust method via PVA deposition, Microsyst Nanoeng 3, 16091 (2017).

As those skilled in the art will appreciate, the body material is typically chosen not to react with (e.g. swell or dissolve when in the presence of) the solvents used in the disclosed methods, and in particular when in contact with the non-aqueous solvent. Accordingly, the material from which the body of the flow chip is constructed typically does not dissolve in and/or swell when contacted with an organic solvent as described herein. For example, PDMS is compatible with solvents such as dimethylcarbonate.

In some embodiments the different portions of the chip beneficially can have different hydrophilicities. For example, as shown in FIG. 13, surfaces of main channel 10 of the flow chip 1 upstream of the extraction phase intersection section 18 are typically hydrophobic (e.g. are manufactured from a hydrophobic material or are treated to be hydrophobic). This can be beneficial as it may prevent the droplet of the pharmaceutical agent in aqueous solvent produced as described herein from wetting the walls of the channels after formation at the solvent phase intersection section 12.

Typically, the extraction solvent is hydrophilic, the surfaces of the extraction phase inlet channels 40 are hydrophilic, and the surfaces of main channel of 10 the flow chip 1 in the extraction phase intersection section 18 and downstream thereof are hydrophilic. This can be beneficial as it may prevent the outer droplet of the non-aqueous solvent (which encapsulates the inner droplet of the aqueous solvent and pharmaceutical agent) from wetting the walls of the channels after formation at the extraction phase intersection section 18.

The flow chip can be produced by any suitable methods for forming three-dimensional structures from solid materials. When the body of the flow chip is made from a polymer moulding (e.g. soft lithography moulding) is a suitable method. Alternatively, the flow chip can be made by hot embossing (e.g. by hot embossing a polymeric sheet). When the body of the flow chip is made from glass, etching is a suitable method. Different portions of the flow cell can be treated with different reagents by using gas (e.g. air) bubbles to isolate regions of the flow cell which should not be treated from the fluid pathway of the cell.

### Aqueous Phase

As explained above, the aqueous phase which is entrained within the solvent phase in accordance with the methods of the present disclosure comprises a pharmaceutical agent and an aqueous solvent.

The aqueous solvent of the aqueous phase may comprise deionised and/or ultrapure water (e.g having a resistivity of at least 10 MΩ cm, such as at least 15 MΩ cm e.g. at least 18 MΩ cm) before addition of any further components e.g. buffer salts.

The aqueous solvent of the aqueous phase typically has a pH of from about 4 to about 10. More usually the aqueous solvent of the aqueous phase has a pH of from about 5 to about 9, such as from about 6 to about 8.

The aqueous solvent of the aqueous phase may optionally comprise one or more buffer salts. Such buffer salts when present are comprised in the aqueous solvent of the aqueous phase. For some applications buffers are not required and the aqueous phase may omit any buffering agent.

When used, preferred buffer salts include Tris; phosphate; citric acid/Na₂HPO₄; citric acid/sodium citrate; sodium acetate/acetic acid; Na₂HPO₄/NaH₂PO₄; imidazole (glyoxaline)/HCl; sodium carbonate/sodium bicarbonate; ammonium carbonate/ammonium bicarbonate; MES; Bis-Tris; ADA; aces; PIPES; MOPSO; Bis-Tris Propane; BES; MOPS; TES; HEPES; DIPSO; MOBS; TAPSO; Trizma; HEPPSO; POPSO; TEA; EPPS; Tricine; Gly-Gly; Bicine; HEPBS; TAPS; AMPD; TABS; AMPSO; CHES; CAPSO; AMP; CAPS and CABS. Further salts include antacid salts such as Mg(OH)₂ and MgCO₃, which are typically immiscible and remain in suspension in the aqueous phase. Preferably, the buffer is pharmaceutically acceptable. Pharmaceutically acceptable buffers include phosphate/phosphoric acid, citrate/citric acid, acetate/acetic acid, histidine, lactate/lactic acid, trometamine, gluconic acid/gluconate, aspartic acid/aspartate, tartaric acid/tartarate, succinic acid/succinate, malic acid/malate, fumaric acid/fumarate and α-ketoglutaric acid/α-ketoglutarate. Most often the buffer salt is selected from phosphate, citrate and acetate.

Selection of appropriate buffers for a desired pH is routine to those skilled in the art, and guidance is available at e.g. http://www.sigmaaldrich.com/life-science/core-bioreagents/biological-buffers/learning-center/buffer-reference-center.html. Buffer salts are preferably used at concentrations of from 1 mM to 1 M, preferably from 1 mM to 100 mM such as about 10 mM to about 50 mM in solution.

A preferred aqueous solvent for use in the aqueous phase of the present disclosure is phosphate buffered saline (PBS). In some embodiments PBS comprises NaCl and/or KCl, buffered to about 7.4 using phosphate buffer. For example, PBS may comprise 137 mM NaCl, 2.7 mM KCl, 8 mM Na₂HPO₄, and 2 mM KH₂PO₄.

In some embodiments the aqueous phase or the aqueous solvent of the aqueous phase comprises one or more gelling agents. This can be beneficial to produce particles in which the aqueous core of the particle is semi-solid, e.g. in the form of a hydrogel. When such gelling agents are absent or are present only at low concentration then the aqueous core may be liquid. Typically, the composition of the aqueous phase is controlled such that the aqueous core is liquid.

When the aqueous phase comprises a gelling agent, any suitable gelling agent may be used. Typically, the gelling agent is pharmaceutically acceptable. Gelling agents that can be used in accordance with the disclosure include tragacanth, pectin, xanathan gum, gellan gum, guar gum, gelatin, starch, carbomers, alginates, carrageenan, chitosan, poloxamer, poly(ethylene) oxide, linear or branched poly(ethylene) glycols (PEG) (e.g. chemically modified poly(ethylene) glycol modified with e.g. acrylate, norbornene, vinyl sulfone, maleimide, and/or thiol functionalities), polycarbophil, gelatin, celluloses (e.g. hydroxypropyl celluylose (HPC), carboxymethylcellulose (CMC), hydroxymethyl cellulose, hydroxypropylmethyl cellulose, hypromellose, hydroxyethyl cellulose (HEC), methylcellulose, etc), povidone, polyvinyl alcohol clays, polymeric gelling agents such as carbomers (e.g. carbomer 934P, carbomer 940, carbomer 941, etc), bentonite, etc.

Typically, when present, a gelling agent is used a concentration of from about 0.1 to about 20% w/v in the aqueous phase, such as at a concentration of from about 0.5% to about 10% w/v, e.g. from about 1% to about 5% w/v. Such concentrations are appropriate to prepare particles in which the aqueous core is a semi-solid.

The aqueous phase may comprise one or more stabilisers such as trehalose and L-lysine.

The aqueous phase may comprise the pharmaceutical agent in solution or suspension. Most usually the aqueous phase comprises the pharmaceutical agent in solution.

Any suitable pharmaceutical agent may be used. Pharmaceutical agents which may be incorporated in the aqueous phase are described in more detail herein.

Typically the pharmaceutical agent is a hydrophilic agent. Typically the pharmaceutical agent is water-soluble. Typically the pharmaceutical agent is soluble in a buffered salt solution having a pH of from about 4 to about 10.

### Solvent Phase

As explained above, the solvent phase entrains the aqueous phase in accordance with the methods of the present disclosure, and comprises a polymer and a non-aqueous solvent. The non-aqueous solvent is capable of dissolving the biodegradable polymer(s) used for the polymeric shell of the particles of the disclosure. The solvent phase thus gives rise to the polymeric shell of the particles produced in accordance with the disclosed methods.

Typically, the non-aqueous solvent is an organic solvent.

Typically, the non-aqueous solvent is biodegradable. An example of a biodegradable solvent is dimethylcarbonate (DMC), which is hydrolysed by water in vitro and metabolised in vivo to methanol and carbon dioxide. DMC is considered to be a green reagent.

Typically, the non-aqueous solvent is capable of dissolving polymers such as aliphatic polyesters, aromatic copolyesters, polyurethanes, polycarbonates, polyamides, poly(ester-amide) s, polyanhydrides, polysaccharides, and blends thereof or copolymers thereof. More typically, the non-aqueous solvent is a solvent for aliphatic polyesters and polyanhydrides.

Typically, the non-aqueous solvent is a solvent for polymers such as poly(lactic-co-glycolic acid) (PLGA), polylactic acid (PLA), polyglycolic acid (PGA), polycaprolactone (PCL), poly(butylene succinate), poly(p-dioxanone) (PPDO), poly(hydroxybutyrate) (PHB), poly(butylene adipate-co-terephtalate) (PBAT), chitosan, cellulose, hyaluronic acid, and blends thereof and copolymers thereof. More typically, the non-aqueous solvent is capable of dissolving poly(lactic-co-glycolic acid) (PLGA), polylactic acid (PLA), polycaprolactone (PCL), and polyanhydrides and blends thereof and copolymers thereof. Still more typically, the non-aqueous solvent is capable of dissolving poly(lactic-co-glycolic acid) (PLGA), polylactic acid (PLA) and polycaprolactone (PCL) and blends thereof and copolymers thereof. Most typically, the non-aqueous solvent is capable of dissolving poly(lactic-co-glycolic acid) (PLGA), polylactic acid (PLA) and polycaprolactone (PCL). For example, the non-aqueous solvent may be capable of dissolving (i.e. acting as a solvent for) PLGA polymers comprising a blend of lactic acid to glycolic acid (L:G ratio) of from 0:100 to 100:0, preferably from 10:90 to 90:10, such as from 25:75 to 75:25, e.g. from 40:60 to 60:40.

Typically, the non-aqueous solvent is not a solvent for the material that the apparatus described herein is made from. This prevents the solvent from affecting the structural integrity of the apparatus during operation of the methods of the disclosure. For example, the non-aqueous solvent is typically not capable of dissolving materials such as PDMS, glass, PMMA, polycarbonate, cyclic olefin copolymer, etc. Most typically the non-aqueous solvent does not dissolve PDMS.

Typically, the non-aqueous solvent is immiscible or at most partially miscible with water and thus is typically immiscible or at most partially miscible with the aqueous phase and/or the extraction phase as described herein. Typically, the non-aqueous solvent has a solubility in water of at most 50 g/100 mL, e.g. at most 30 g/100 mL, such as at most 20 g/100 mL, e.g. at most 15 g/100 mL, more preferably at most 10 g/100 mL or less. For example, DMC has a solubility of 13.9 g/100 mL.

Typically, the non-aqueous solvent has a melting point of less than about 30° C., e.g. less than about 25° C., such as less than 20° C. e.g. less than 15° C., more preferably less than 10° C. This ensures that the solvent is not solid under typical operation temperatures. Typically, the non-aqueous solvent has a boiling point of at least about 50° C., such as at least 60° C., e.g. at least 70° C., e.g. at least 80° C., e.g. at least 90° C. or more. This ensures that the solvent is liquid under typical operation temperatures. For example, DMC has a melting point of about 2-4° C. and a boiling point of about 90° C.

Typically, the non-aqueous solvent is extractable into an aqueous solvent. By extracting the non-aqueous solvent into an aqueous solvent the polymer dissolved therein solidifies forming a solid homogeneous shell around the aqueous core of the particle. Alternatively or additional, the non-aqueous solvent may be removable by evaporation.

Exemplary solvents for use in accordance with the present disclosure include dimethyl carbonate (DMC), dichloromethane (DCM), toluene and/or chloroform. Other solvents include n-hexane, diethyl ether, benzene, n-butanol, butyl acetate, carbon tetrachloride, cyclohexane, 1,2-dichloroethane, ethyl acetate, heptane, methyl-t-butyl ether, methyl ethyl ketone, pentane, and dicholoroethylene. More typically, the solvent used is dimethyl carbonate (DMC).

It is within the capacity of one skilled in the art to select an appropriate solvent for a desired polymer to be comprised in the shell of the particles produced herein: this is an operational parameter which can be determined by the skilled user.

Typically, the concentration of polymer in the non-aqueous solvent is high. Typically, the concentration is as high as possible whilst being capable of being processed by the device and in accordance with the methods disclosed herein. The use of high polymer concentrations can be useful to produce particles having thick shells, which in turn can improve the stability of the particles produced in the disclosed methods, e.g. in storage and/or once administered to a subject (e.g. to delay release of the aqueous core). Any suitable concentration can be used in the disclosure. For example, the concentration of the polymer in the non-aqueous solvent may range from about 0.5% to about 25% w/v (weight to volume), e.g. from about 5% to about 20% w/v, for example from about 8% to about 18% w/v such as from about 10 to about 15% w/v e.g. about 12.5% w/v. Typically, higher concentrations are useful with polymers of lower molecular weight, and lower concentrations are useful with polymers of higher molecular weight. For example, a polymer (e.g. PLGA) having a molecular weight of from about 5 kDa to about 20 kDa (e.g. from about 7 kDa to about 17 kDa) may be used at a concentration of from about 15% to about 20% w/v, e.g about 17.5% w/v. A polymer (e.g. PLGA) having a molecular weight of from about 20 kDa to about 40 kDa (e.g. from about 24 kDa to about 38 kDa) may be used at a concentration of from about 10% to about 15% w/v, e.g about 13% w/v. A polymer (e.g. PLGA) having a molecular weight of from about 40 kDa to about 55 kDa (e.g. from about 38 kDa to about 54 kDa) may be used at a concentration of from about 8% to about 12% w/v, e.g about 10.5% w/v. A polymer (e.g. PLGA) having a molecular weight of from about 55 kDa to about 70 kDa (e.g. from about 54 kDa to about 69 kDa) may be used at a concentration of from about 5% to about 10% w/v, e.g about 8.5% w/v.

Typically, the solvent phase comprises one or more biodegradable polymers. Any suitable biodegradable polymer can be used in accordance with the invention. The use of biodegradable polymers is highly advantageous as it prevents build-up of non-biodegradable polymers in the body when particles produced in accordance with the disclosed methods are administered to a subject, which can be toxic or harmful upon accumulation.

Typically, the one or more biodegradable polymers are capable of biodegrading once administered to a subject as described herein in a timeframe of between about 1 day and about 1 year, such as from about 1 week to about 6 months, e.g. from about 2 weeks to about 4 months (e.g. from about 2 weeks to about 10, 11, 12, 13 or 14 weeks) e.g. from about 2 weeks to about 2 months. It is straightforward for one of skill in the art to determine the timeframe for biodegradability of a polymer.

Typically, a biodegradable polymer suitable for use in accordance with the disclosure has a melting temperature (Tm) of from about 20 to about 200° C., e.g. from about 50 to about 150° C. This can facilitate maintaining compositions comprising the polymer in a sterile form.

Typically, biodegradable polymers suitable for use in accordance with the present disclosure are soluble in organic solvents such as dimethyl carbonate (DMC), dichloromethane (DCM), toluene and/or chloroform. Usually biodegradable polymers suitable for use in accordance with the present disclosure are soluble in dimethyl carbonate (DMC). This can facilitate facile manufacturing in accordance with the methods and devices disclosed herein.

Typically, the methods and resulting particles provided herein comprise just one type of polymer, or comprise at most a mixture of two or three polymers. More typically, just one type of polymer is used. Use of a single type of polymer can facilitate facile manufacturing in accordance with the methods and devices disclosed herein. Use of a mix or blend or polymers or of a copolymer can allow for improved tuneability of the release profile of the particles generated in accordance with the disclosed methods.

Typically, biodegradable polymers suitable for use in accordance with the present disclosure are selected from aliphatic polyesters, aromatic copolyesters, polyurethanes, polycarbonates, polyamides, poly(ester-amide) s, polyanhydrides, polysaccharides, and blends thereof or copolymers thereof.

More typically, biodegradable polymers suitable for use in accordance with the present disclosure are selected from aliphatic polyesters and polyanhydrides.

Typically, biodegradable polymers suitable for use in accordance with the present disclosure are selected from poly(lactic-co-glycolic acid) (PLGA), polylactic acid (PLA), polyglycolic acid (PGA), polycaprolactone (PCL), poly(butylene succinate), poly(p-dioxanone) (PPDO), poly(hydroxybutyrate) (PHB), poly(butylene adipate-co-terephtalate) (PBAT), chitosan, cellulose, hyaluronic acid, and blends thereof and copolymers thereof.

More typically, biodegradable polymers suitable for use in accordance with the present disclosure are selected from poly(lactic-co-glycolic acid) (PLGA), polylactic acid (PLA), polycaprolactone (PCL), and polyanhydrides and blends thereof and copolymers thereof. Still more typically, biodegradable polymers suitable for use in accordance with the present disclosure are selected from poly(lactic-co-glycolic acid) (PLGA), polylactic acid (PLA) and polycaprolactone (PCL) and blends thereof and copolymers thereof. Most typically, biodegradable polymers suitable for use in accordance with the present disclosure are selected from poly(lactic-co-glycolic acid) (PLGA), polylactic acid (PLA) and polycaprolactone (PCL).

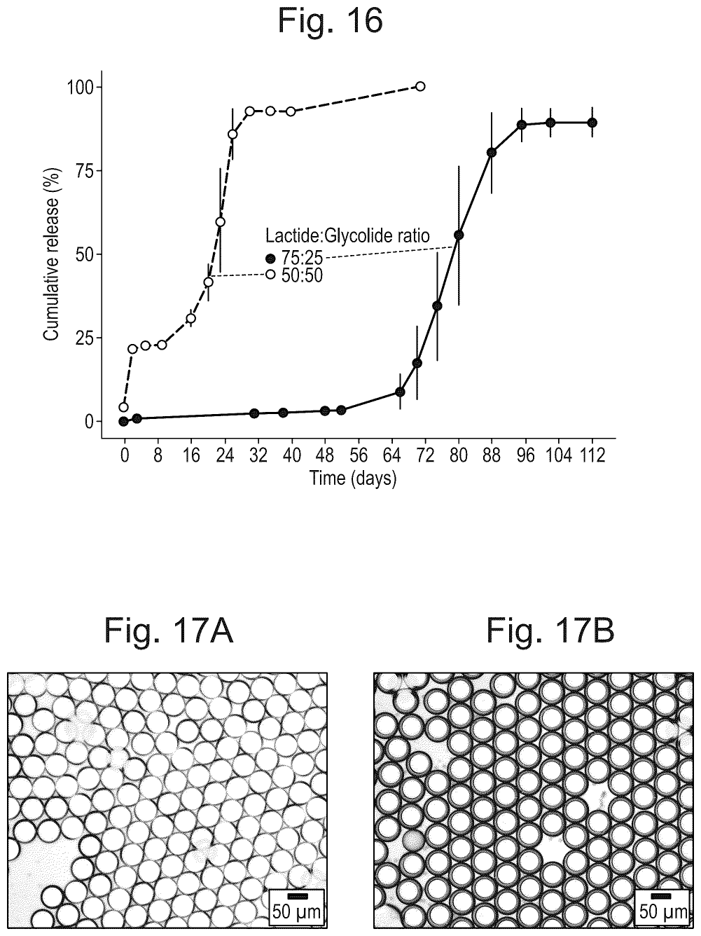

More preferably, the biodegradation polyester is poly(lactic-co-glycolic acid) (PLGA), or a blend or copolymer thereof. Preferably the PLGA polymer comprises a blend of lactic acid to glycolic acid (L:G ratio) of from 0:100 to 100:0, preferably from 10:90 to 90:10, such as from 25:75 to 75:25, e.g. from 40:60 to 60:40. Without being bound by theory, it is believed that release of the liquid core from the solid shell of the particles of the present disclosure may be retarded when PLGA polymers are used having higher L:G ratios. Accordingly, in some embodiments the L:G ratio is from 50:50 to 100:0, such as from 60:40 to 90:10, e.g. from about 70:30 to about 80:20 e.g. about 75:25. Accordingly, those skilled in the art can control the release profile of the particles of the present disclosure by controlling the L:G ratio of a PLGA polymer used to form the polymeric shell of the particles. This is described in example 4 and FIG. 16.

Typically, biodegradable polymers suitable for use in accordance with the present disclosure have an average molecular weight (e.g. a weight-average molecule weight) of from about 1 kDa to about 250 kDa, e.g. from about 5 kDa to about 100 kDa e.g. from about 10 kDa to about 50 kDa. The inventors have found that the release profile of the particles produced in accordance with the disclosed methods can be tuned by controlling the average molecular weight of the polymer used to produce the polymeric shell. Typically, the use of a biodegradable polymer having a higher average molecular weight leads to a longer delay before release of the pharmaceutical agent from the liquid core of the polymer. Delay in this context may be delay in vivo following administration of a composition comprising the particles to a subject, or may be determined or measured in vitro.

For example, the use of PLGA having an average molecular weight of from about 5 to 20 kDa may result in particles which release the core contents after about 15 to 30 days when measured in vitro. The use of PLGA having an average molecular weight of from about 20 to 40 kDa may result in particles which release the core contents after about 16 to 35 days when measured in vitro. The use of PLGA having an average molecular weight of from about 40 to 55 kDa may result in particles which release the core contents after about 20 to 40 days when measured in vitro. The use of PLGA having an average molecular weight of from about 55 to 70 kDa may result in particles which release the core contents after about 25 to 45 days when measured in vitro.

By way of a particularly preferred example, the solvent phase may thus comprise a non-aqueous solvent which is DMC, and a polymer which is PLGA having an average molecular weight of from about 5 to 20 kDa, preferably in a concentration of from about 5% to about 20% w/v, such as from about 12.5% to about 17.5% w/v.

### Extraction Phase

As explained herein, the disclosed methods comprise forming droplets comprising the aqueous phase encapsulated by the solvent phase in the extraction phase; and extracting the non-aqueous solvent of the solvent phase thereby producing particles.

The extraction phase comprises an extraction solvent. Any suitable extraction solvent may be used. A suitable extraction solvent is a solvent capable of extracting the non-aqueous solvent from the solvent phase of the droplet emulsion as discussed above.

Typically, the extraction solvent is non-organic. Typically, the extraction solvent is aqueous, although other polar solvents such as ionic liquids can also be used.

Typically, the extraction solvent is physiologically acceptable. This is beneficial as it means that trace amounts of the solvent may be retained in the final particle product (e.g. as a medium in which the particles are maintained) and may be administered to a subject without harm.

The extraction solvent may comprise deionised and/or ultrapure water (e.g having a resistivity of at least 10 MΩ cm, such as at least 15 MΩ cm e.g. at least 18 MΩcm) before addition of any further components e.g. buffer salts.

The extraction solvent typically has a pH of from about 4 to about 10. More usually the extraction solvent has a pH of from about 5 to about 9, such as from about 6 to about 8.

The extraction solvent may optionally comprise one or more buffer salts. For some applications buffers are not required and the extraction solvent may omit any buffering agent. When used, preferred buffer salts include Tris; phosphate; citric acid/Na₂HPO₄; citric acid/sodium citrate; sodium acetate/acetic acid; Na₂HPO₄/NaH₂PO₄; imidazole (glyoxaline)/HCl; sodium carbonate/sodium bicarbonate; ammonium carbonate/ammonium bicarbonate; MES; Bis-Tris; ADA; aces; PIPES; MOPSO; Bis-Tris Propane; BES; MOPS; TES; HEPES; DIPSO; MOBS; TAPSO; Trizma; HEPPSO; POPSO; TEA; EPPS; Tricine; Gly-Gly; Bicine; HEPBS; TAPS; AMPD; TABS; AMPSO; CHES; CAPSO; AMP; CAPS and CABS. Preferably, the buffer is pharmaceutically acceptable.

Pharmaceutically acceptable buffers include phosphate/phosphoric acid, citrate/citric acid, acetate/acetic acid, histidine, lactate/lactic acid, trometamine, gluconic acid/gluconate, aspartic acid/aspartate, tartaric acid/tartarate, succinic acid/succinate, malic acid/malate, fumaric acid/fumarate and α-ketoglutaric acid/α-ketoglutarate. Most often the buffer salt is selected from phosphate, citrate and acetate.

Selection of appropriate buffers for a desired pH is routine to those skilled in the art, and guidance is available at e.g. http://www.sigmaaldrich.com/life-science/core-bioreagents/biological-buffers/learning-center/buffer-reference-center.html. Buffer salts are preferably used at concentrations of from 1 mM to 1 M, preferably from 1 mM to 100 mM such as about 10 mM to about 50 mM in solution.

A preferred extraction solvent is phosphate buffered saline (PBS). In some embodiments PBS comprises NaCl and/or KCl, buffered to about 7.4 using phosphate buffer. For example, PBS may comprise 137 mM NaCl, 2.7 mM KCl, 8 mM Na₂HPO₄, and 2 mM KH₂PO₄.

The extraction solvent may comprise a surfactant. A surfactant may be used to assist in preventing the coalescence droplets without perturbing the emulsification process.

Any suitable surfactant may be used. Typically the surfactant is water-soluble and physiologically acceptable. The use of physiologically acceptable surfactants is advantageous as it can in some embodiments avoid the need to remove all traces of the surfactant from the particles prior to their administration to a subject.

Typically, the surfactant is selected from anionic surfactants, cationic surfactants, amphoteric surfactants and non-ionic surfactants. Anionic surfactants are typically organic salts which dissociate at high pH to form a long-chain anion with surface activity. Anionic surfactants typically contain carboxylate, sulfonate, phosphate or sulfate groups. Examples include alkyl sulfates such as ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), and sodium myreth sulfate. Other anionic surfactants include docusate (dioctyl sodium sulfosuccinate), perfluorooctanesulfonate (PFOS), perfluorobutanesulfonate, alkyl-aryl ether phosphates, alkyl ether phosphates and carboxylates such as sodium stearate, sodium lauroyl sarcosinate, perfluorononanoate, and perfluorooctanoate (PFOA or PFO).

Cationic surfactants are typically positively charged substances that also generally have antimicrobial properties. Examples include phosphatidylcholine, octenidine dihydrochloride, permanently charged quaternary ammonium salts: cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB).

Amphoteric surfactants exhibit both anionic and cationic dissociations and include betaines e.g. sulfobetaine and natural substances such as amino acids and phospholipids. Other examples include sulfonates, as in the sultaines CHAPS (3-[(3-cholamidopropyl) dimethylammonio]-1-propanesulfonate) and cocamidopropyl hydroxysultaine, betaines such as cocamidopropyl betaine have a carboxylate with the ammonium, and phospholipids such as phosphatidylserine, phosphatidylethanolamine, phosphatidylcholine, and sphingomyelins. Lauryldimethylamine oxide and myristamine oxide are two further common examples.

Non-ionic surfactants include esters such as Polyoxyethylene sorbitan fatty acid esters (e.g. Polysorbate, Tween®), Polyoxyethylene 15 hydroxy stearate (e.g. Macrogol 15 hydroxy stearate, Solutol HS15®), Polyoxyethylene castor oil derivatives (e.g Cremophor® EL, ELP, RH 40), Polyoxyethylene stearates (e.g. Myrj®), Sorbitan fatty acid esters (e.g. Span®), Polyoxyethylene alkyl ethers (e.g. Brij®), and Polyoxyethylene nonylphenol ethers (e.g. Nonoxynol®)

Further examples include ethoxylates (e.g. fatty alcohol ethoxylates, narrow-range ethoxylate, octaethylene glycol monododecyl ether, pentaethylene glycol monododecyl ether); alkylphenol ethoxylates (APEs or APEOs) (e.g. nonoxynols and Triton X-100); fatty acid ethoxylates; ethoxylated amines and/or fatty acid amides (e.g. polyethoxylated tallow amine; cocamide monoethanolamine; cocamide diethanolamine); terminally blocked ethoxylates such as poloxamers; fatty acid esters of polyhydroxy compounds; fatty acid esters of glycerol (e.g. glycerol monostearate, glycerol monolaurate); fatty acid esters of sorbitol (e.g. Spans such as sorbitan monolaurate, sorbitan monostearate and sorbitan tristearate; Tweens such as Tween 20, Tween 40, Tween 60, Tween 80); Fatty acid esters of sucrose′ and Alkyl polyglucosides (e.g. decyl glucoside, lauryl glucoside, octyl glucoside).

A particularly suitable surfactant for use in the present disclosure is polyvinyl alcohol (PVA). Typically, the PVA may have a molecular weight of from about 2000 to about 20,000 g/mol, such as from about 5,000 to about 15,000 g/mol, e.g. from about 8,000 to about 12,000 g/mol, e.g. from about 9,000 to about 10,000 g/mol. Typically the PVA may be about 50 to about 99% hydrolyzed, such as from about 60 to about 90% hydrolyzed, e.g. from about 70 to about 85% hydrolyzed such as about 80% hydrolyzed.

The surfactant may be used at a concentration in the extraction solvent of from about 0.01% to about 20% w/v, e.g from about 0.1% to about 10% w/v, e.g. from about 1% to about 5% w/v such as about 3% w/v.

In an exemplary embodiment, the surfactant may be PVA having a molecular weight of about 9,000-10,000 g/mol; about 80% hydrolyzed; at a concentration of about 3% w/v in the extraction solvent. The extraction solvent may for example be PBS as defined herein.

Further components may be incorporated in the extraction solvent. For example, viscoenhancers such as glycerol, poly(ethylene) glycol, carboxymethyl cellulose, xanthan gum, etc can be incorporated. Any of the gelling agents disclosed in the context of the aqueous phase can typically be included in the extraction solvent, usually at low concentrations below their gelling concentration.

Osmolarity regulators such as salts may be further included. Examples include NaCl, KCl, MgCl₂, phosphates, etc. In general, any of the buffer salts disclosed herein may be used to regulate the osmolarity of the extraction solvent. This can be useful to match the osmolarity of the aqueous core of the particles.

### Particles

It will be appreciated from the above discussion that the particles produced in accordance with the present disclosure may be contrasted against particles produced by conventional double emulsion procedures, such as by sonication of an aqueous solution of a pharmaceutical agent in an organic polymer solution to form a water-in-oil (W/O) emulsion followed by the further combination and homogenisation of such an emulsion in a further aqueous solution to form a water-in-oil-in-water (W/O/W) double emulsion. As set out in the examples, particles formed by such methods are typically not monodisperse, spherical, and/or robust and typically do not have the well-defined aqueous core encapsulated by a solid polymeric shell, which characterises the particles of the present disclosure.

As will be apparent, the aqueous core is derived from the aqueous phase discussed herein. Accordingly, the composition of the aqueous core of the particles provided herein may be controlled by controlling the composition of the aqueous phase used in said apparatus and methods, when such particles are produced using the apparatus and/or methods disclosed herein. In some embodiments the composition of the aqueous core the same as the composition of the aqueous phase. In some embodiments the components of the aqueous phase are identical to the components of the aqueous core of the particles.

Accordingly, the aqueous core may be liquid, or semi-solid in the form of a hydrogel. When the aqueous core is a gel the aqueous core typically comprises one or more of the gelling agents described in more detail herein in the context of the aqueous phase. Typically, the aqueous core is liquid.

The aqueous core may comprise the pharmaceutical agent in solution or suspension. Most usually the aqueous core comprises the pharmaceutical agent in solution. Pharmaceutical agents are described in more detail herein.

The aqueous core typically has a pH of from about 4 to about 10 and may comprise one or more buffer salts as discussed in more detail herein, particularly as discussed in the context of the aqueous phase.

As will be apparent from the above discussion, the methods provided herein yield homogeneous particles in which the aqueous core is well defined.

Typically, the mean diameter of the aqueous core of the particles of the present disclosure is from about 1 μm to about 300 μm. More typically, the mean diameter of the core of the particles of the present disclosure is from about 3 μm to about 150 μm, e.g. from about 10 μm to about 100 μm, such as from about 30 μm to about 70 μm e.g. from about 40 μm to about 55 μm. Many methods are available for measurement of the diameter of the core of liquid/shell particles, for example SEM and optical (e.g. fluorescence) microscopy.

Typically, the aqueous core is highly spherical. This is defined in more detail herein. Preferably, the smallest diameter of the aqueous core is at least 70% of the largest diameter of the aqueous core. More typically, the smallest diameter of the aqueous core may be at least 90% of the largest diameter of the aqueous core, such as at least 95%, at least 96%, at least 97%, at least 98%, at least 99%, at least 99.9%, at least 99.99% or at least 99.999% of the largest diameter of the aqueous core. Preferably, the smallest diameter of the aqueous core is indistinguishable from the largest diameter of the aqueous core when measured e.g. by scanning electron microscopy (SEM) or optical microscopy (e.g. fluorescence microscopy).

The polymeric shell is derived from the polymer(s) in the solvent phase discussed above. Accordingly, the composition of the polymeric shell of the particles provided herein may be controlled by controlling the composition of the solvent phase used in the disclosed methods, when such particles are produced using such methods. In some embodiments the composition of the polymeric shell may be the same as the non-solvent components of the solvent phase. In some embodiments the non-solvent components of the solvent phase are identical to the components of the polymeric shell of the particles.

For example, the particle may comprise a solid polymeric shell comprising one or more biodegradable polymers selected from poly(lactic-co-glycolic acid) (PLGA), polylactic acid (PLA), polyglycolic acid (PGA), polycaprolactone (PCL), poly(butylene succinate), poly(p-dioxanone) (PPDO), poly(hydroxybutyrate) (PHB), poly(butylene adipate-co-terephtalate) (PBAT), chitosan, cellulose, hyaluronic acid, and blends thereof and copolymers thereof, for example, PLGA. The L:G ratio of the PLGA may be chosen as discussed above. For example, the particle may comprise a solid polymeric shell comprising PLGA having an average molecular weight of from about 5 to about 100 kDa. The molecular weight can be chosen or determined according to the desired release profile of the particles, as described above.

Typically, the particles produced in accordance with the disclosure have a polymeric shell having a thickness of from about 0.1 μm to about 100 μm, for example from about 1 μm to about 50 μm, such as from about 3 μm to about 20 μm e.g. from about 5 to about 15 μm, such as about 10 μm. The thickness of the polymeric shell is controllable as disclosed herein (e.g. by controlling the concentration of the polymer in the solvent phase and/or the flow rates used in the disclosed methods, as discussed above) and allows the release profile of the particle to be precisely tuned. The thickness of a polymeric shell of a polymer can be readily determined, e.g. by SEM or TEM, or by optical (e.g. fluorescence) microscopy.

Typically, the polymeric shell of the particles of the present disclosure is highly uniform. Preferably, the thickness of the thinnest part of the polymeric shell is at least 70% of the thickness of the thickest part of the polymeric shell. More typically, the thickness of the thinnest part of the polymeric shell may be at least 90% of the thickness of the thickest part of the polymeric shell, such as at least 95%, at least 96%, at least 97%, at least 98%, at least 99%, at least 99.9%, at least 99.99% or at least 99.999% of the thickness of the thickest part of the polymeric shell. Preferably, the thickness of the thinnest part of the polymeric shell is indistinguishable from the thickness of the thickest part of the polymeric shell when measured e.g. by scanning electron microscopy (SEM) or optical microscopy (e.g. fluorescence microscopy).

It is within the capacity of one skilled in the art to modify the thickness of the shell according to the application intended for the particle: this is an operational parameter which can be determined by the skilled user.

The particles of the present disclosure are spherical, uniform, and robust.

Typically, the mean diameter the particles of the present disclosure is from about 5 μm to about 500 μm. More typically, the mean diameter the particles of the present disclosure is from about 10 μm to about 250 μm, such as from about 20 μm to about 150 μm, e.g. from about 40 μm to about 100 μm, such as from about 50 μm to about 80 μm e.g. from about 55 μm to about 70 μm.

Many methods are available for measurement of particle diameter, for example SEM. dynamic light scattering and optical (e.g. fluorescence) microscopy.

The particles of the present disclosure are highly spherical. Those skilled in the art will appreciate that particles made by previously known methods typically deviate significantly from sphericity.

A mathematically perfect sphere may be considered as having a single diameter regardless of the angle, orientation or plane of the vector defining that diameter. In practice, physical objects inevitably deviate from mathematically perfect sphericity. Accordingly, a parameter for sphericity of the particles of the present disclosure can be defined by comparing the smallest diameter that can be measured for the particle to the largest diameter that can be measured.

Preferably, the smallest diameter of the particle is at least 70% of the largest diameter of the particle. More typically, the smallest diameter of the particle may be at least 90% of the largest diameter of the particle, such as at least 95%, at least 96%, at least 97%, at least 98%, at least 99%, at least 99.9%, at least 99.99% or at least 99.999% of the largest diameter of the particle. Preferably, the smallest diameter of the particle is indistinguishable from the largest diameter of the particle when measured e.g. by scanning electron microscopy (SEM) or optical (e.g. fluorescence) microscopy.

The particles of the present disclosure are typically monodisperse. Typically the polydispersity index (PDI) of the particles of the invention, or of a composition comprising the particles of the invention, is 0.3 or less, preferably 0.2 or less, more preferably 0.1 or less.

Dispersity in a sample of particles of the present disclosure can also be defined in terms of the coefficient of variation of the mean diameters of the particles in the sample (where the coefficient of variation, CV, is defined as CV (%)={standard deviation of diameters}/{mean of diameters}×100). Typically, said coefficient of variation is between about 0.1% and 20%, such as between about 1% and 15%, e.g. from about 4% to about 10%.

Typically, the particles of the present disclosure have a well-defined release profile.

The release profile is typically sigmoidal in nature, as discussed in the examples. Thus, the release of the aqueous core from the particles arises in three stages: (i) an initial plateau timeframe in which there is very little (if any) release of the aqueous core from the particles, constituting an initial “delay” phase; (ii) a release stage wherein the aqueous core is released and (iii) a later plateau following release of at least the majority of the aqueous cores from the particles in the sample.

The timescales of each of these three stages—and in particular stages (i) and (ii)-can be controlled by the user of the disclosed methods: they are parameters that can be determined by selection of e.g. the size and composition of the aqueous phase which corresponds to the aqueous core of the particles once produced; the thickness and composition of the polymeric shell; and the nature of the therapeutic agent (if any) encapsulated in the core. It is within the capacity of one skilled in the art to modify the components of the particles in order to arrive at desired release kinetics. For example:
Typically, increasing the molecular weight of the polymer comprised in the solvent phase and thus comprised in the polymeric shell of the resultant particles increases the timeframe of the initial plateau region and thus delays release of the aqueous core from the polymeric shell.
Typically, increasing the thickness of the polymeric shell was surprisingly shown to usually reduce the timeframe of the initial plateau region and thus speed up release of the aqueous core from the polymeric shell. Without being bound by theory, the inventors consider that this may arise as thinner shells do not degrade by autocatalysis.
Typically, altering the composition of the polymer comprised in the solvent phase and thus comprised in the polymeric shell of the resultant particles alters the timeframe of the initial plateau region and thus rate of release of the aqueous core from the polymeric shell. For example, when a PLGA polymer is used as described herein, increasing the L:G ratio typically increases the timeframe of the initial delay plateau and thus delays release of the aqueous core from the polymeric shell.

Typically, the initial plateau region may last for between about 1 day to about 1 year, such as from about 1 week to about 6 months, e.g. from about 2 weeks to about 4 months (e.g. from about 2 weeks to about 10, 11, 12, 13 or 14 weeks), e.g. from about 2 weeks to about 2 months. Typically, the release stage has a duration of from about 1 hour to about 3 weeks, such as from about 1 day to about 5 days such as about 2-3 days. The final plateau region is typically indefinite in length; it may be for example at least 1 day, at least 1 week; at least 1 month; at least one year or more.

### Pharmaceutical Agents

As discussed above, the aqueous phase and thus the aqueous core of the provided particles comprise a pharmaceutical agent. Additional pharmaceutical agents may be present in a pharmaceutical composition comprising the particles and one or more further components. A pharmaceutical agent may be present in the core of the particles and in a pharmaceutical composition comprising the particles. A pharmaceutical agent may be comprised in the core of the particles and a second pharmaceutical agent may be comprised in a pharmaceutical composition comprising the particles.

Examples of pharmaceutical agents which may be comprised in the aqueous phase and thereby contained in the core of the particles of the present disclosure and/or in a composition comprising such particles include: proteins and other biomacromolecules, water miscible drugs, virus-like particles, viruses, nanometer-sized particles, therapeutic bacteria, therapeutic cells, polysaccharides, etc.

Examples of pharmaceutical agents which may be contained in the core of the particles of the present disclosure and/or in a composition comprising such particles include: immunogenic agents, analgesics, antibiotics, anti-thrombotic drugs (such as t-PA), antidepressants, anticancer drugs such as chemotherapy drugs including combinations thereof (e.g. 5-fluorouracil, folinic acid (FA) or salts thereof, 6-mercaptopurine, cytarabine, gemcitabine, oxaliplatin, methotrexate, actinomycin-D, bleomycin, daunorubicin, doxorubicin, docetaxel, estramustine, paclitaxel, vinblastine, etoposide, irinotecan, teniposide, topotecan, prednisone, methylprednisolone, dexamethasone and combinations of two or more of these chemotherapy drugs), antiepileptics, anti-inflammatory drugs, antipsychotic agents, antivirals, sedatives, steroids, antidiabetics, cardiovascular drugs, and drugs for pain management, treatment of skin conditions and treatment of brain diseases. In some embodiments the pharmaceutical agent is an immunogenic agent. An immunogenic agent may be used as a vaccine, e.g. a vaccine for use in prevention or treatment (typically prevention) of a disorder or condition herein.

A pharmaceutical agent used in accordance with the present disclosure may comprise or consist of a therapeutic saccharide such as D-mannose, L-frucose, D-galactose, N-acetyl-D-glucosamine, sialic acid, N-acetyl-D-mannosamine, ribitol, etc. Dextran molecules are further examples of therapeutic saccharides.

The particles of the present disclosure and compositions comprising such particles may also be used to deliver diagnostic agents including contrast agents and radioactive or tracer molecules. Particular examples of useful contrast agents are MRI contrast agents such as a Gd-based MRI contrast agents, or fluoroscopy contrast agents. Accordingly, such agents may also be comprised in the aqueous phase and thereby in the aqueous core.

The particles of the present disclosure and compositions comprising such particles are particularly useful for delivery of vaccines. The particles of the present disclosure and compositions comprising such particles are especially useful for delivery of vaccines for the prevention (prophylaxis) of disorders described herein. Examples of suitable compositions of polypeptide fragments and methods of administration are provided in Esseku and Adeyeye (2011) and Van den Mooter G. (2006). Vaccine and immunotherapy composition preparation is generally described in Vaccine Design (“The subunit and adjuvant approach” (eds Powell M. F. & Newman M. J. (1995) Plenum Press New York).

A pharmaceutical agent used in accordance with the present disclosure may comprise or consist of a non-immunomodulatory polypeptide (or non-immunomodulatory protein) is a polypeptide (or protein) which does not give rise to an immune response on administration. Typically, such a polypeptide or protein is one which occurs naturally within the subject. An example of a non-immunomodulatory protein in the case of a human subject is human serum albumin. More generally, human blood proteins are examples of non-immunomodulatory proteins, including insulin, globulin and haemoglobin.

A pharmaceutical agent used in accordance with the present disclosure may comprise or consist of an immunomodulatory polypeptide (or immunomodulatory protein). An immunomodulatory polypeptide (or immunomodulatory protein) is a polypeptide (or protein) which induces an immune response on administration to a subject. Typically, the subject is a human and the protein is one which induces an immune response on administration to a human subject. Typically, administration of an immunomodulatory polypeptide (or immunomodulatory protein) causes the subject to raise antibodies against the polypeptide (or protein), although alternative immune responses may arise as well as or instead of raising antibodies.

The immunomodulatory polypeptide may be, for example, an immune checkpoint modulator, an antibody, a vaccine antigen, an adjuvant or an anti-inflammatory polypeptide. Further examples of immunomodulatory polypeptides useful in the present invention include macrophage colony stimulating factor (M-CSF), tumour necrosis factor (TNF. e.g. TNF-alpha), granulocyte macrophage colony stimulating factor (GM-CSF) and interferons.

Examples of suitable immune checkpoint modulators include anti-PDL-1 (aPDL1), anti-PD1 (aPD1), anti-CTLA4 (aCTLA4), bi specific T-cell engagers (BiTE), cytokines and chemokines such as interleukins (e.g. IL-1, IL-2, IL-4, IL-5, IL-6, IL-7, IL-10, IL-12, etc.).

Examples of suitable antibodies include aPDL1; aPD1; aCTLA4; BiTE antibodies; anti growth factor antibodies that also trigger antibody dependent cellular cytotoxicity (ADCC) such as anti-EGFR antibodies (e.g. cetuximab and Herceptin); antibodies having anti-inflammatory effect such as anti-TNF (adalimumab, Humira); and JAK-inhibitor antibodies.

Examples of suitable antigens include vaccine antigens. For example, at least one pathogenic antigen protein may be used. The pathogenic antigen protein may be a full length protein or a fragment thereof. Examples of suitable antigen proteins include fragments of viral proteins or of parasitic proteins. The antigen may from a pathogen selected from Cal09 (flu), a coronavirus (e.g. Beta-coronaviridae or SARS-COV-2), hepatitis B, Bordatella pertussis (whooping cough), Streptococcus pneumoniae, a meningococcus bacterium, human papilloma virus (HPV), or Plasmodium sporozoites (malaria parasite).

The antigen protein may be a viral spike protein, preferably a spike protein of the SARS-COV-2 protein (e.g. SARS-COV-2, S1 subunit protein (RBD)). In another aspect, the antigen is a circumsporozoite protein (CSP), a secreted surface protein of the sporozoite parasite. In a further aspect, the antigen is HepB surface antigen protein (HBsAg). In a further aspect, the antigen is associated with the influenza virus and is selected from haemagglutinin and/or an influenza neuraminidase. In another aspect, the antigen is filamentous haemagglutinin (pertussis). In another aspect, the antigen is pneumococcal surface protein A (PspA). In another aspect, the antigen is selected from Neisserial adhesin A (NadA), Neisserial Heparin Binding Antigen (NHBA) and/or factor H binding protein (fHbp). In another aspect, the antigen is an HPV-16 E6/E7 fusion protein. Fragments or genetically modified versions of any of these proteins may also be used.

A pharmaceutical agent used in accordance with the present disclosure may comprise or consist of a nucleic acid vaccine. In some embodiments, the nucleic acid vaccine is a DNA vaccine. In some embodiments, DNA vaccines, or gene vaccines, comprise a plasmid with a promoter and appropriate transcription and translation control elements and a nucleic acid sequence encoding one or more polypeptides of the disclosure. In some embodiments, the plasmids also include sequences to enhance, for example, expression levels, intracellular targeting, or proteasomal processing. In some embodiments, DNA vaccines comprise a viral vector containing a nucleic acid sequence encoding one or more polypeptides. In additional aspects, the compositions disclosed herein comprise one or more nucleic acids encoding peptides determined to have immunoreactivity with a biological sample. For example, in some embodiments, the compositions comprise one or more nucleotide sequences encoding 1, 2, 3, 4, 5, 6, 7, 8, 9, 10, 11, 12, 13, 14, 15, 16, 17, 18, 19, 20, or more peptides comprising a fragment that is a T cell epitope capable of binding to at least three HLA class I molecules and/or at least three or four HLA class II molecules of a patient. In some embodiments, the peptides are derived from an antigen that is expressed in cancer. In some embodiments the DNA or gene vaccine also encodes immunomodulatory molecules to manipulate the resulting immune responses, such as enhancing the potency of the vaccine, stimulating the immune system or reducing immunosuppression. Strategies for enhancing the immunogenicity of DNA or gene vaccines include encoding of xenogeneic versions of antigens, fusion of antigens to molecules that activate T cells or trigger associative recognition, priming with DNA vectors followed by boosting with viral vector, and utilization of immunomodulatory molecules.

A pharmaceutical agent used in accordance with the present disclosure may comprise or consist of an RNA vaccine. In some embodiments, the RNA is non-replicating mRNA or virally derived, self-amplifying RNA. In some embodiments, the non-replicating mRNA encodes the peptides disclosed herein and contains 5′ and 3′ untranslated regions (UTRs). In some embodiments, the virally derived, self-amplifying RNA encodes not only the peptides disclosed herein but also the viral replication machinery that enables intracellular RNA amplification and abundant protein expression. In some embodiments, the RNA is directly introduced into the individual. In some embodiments, the RNA is chemically synthesized or transcribed in vitro. In some embodiments, the mRNA is produced from a linear DNA template using a T7, a T3, or an Sp6 phage RNA polymerase, and the resulting product contains an open reading frame that encodes the peptides disclosed herein, flanking UTRs, a 5′ cap, and a poly(A) tail. In some embodiments, various versions of 5′ caps are added during or after the transcription reaction using a vaccinia virus capping enzyme or by incorporating synthetic cap or anti-reverse cap analogues. In some embodiments, an optimal length of the poly(A) tail is added to mRNA either directly from the encoding DNA template or by using poly(A) polymerase. The RNA may encode one or more peptides comprising a fragment that is a T cell epitope capable of binding to at least three HLA class I and/or at least three or four HLA class II molecules of a patient. In some embodiments, the fragments are derived from an antigen that is expressed in cancer. In some embodiments, the RNA includes signals to enhance stability and translation. In some embodiments, the RNA also includes unnatural nucleotides to increase the half-life or modified nucleosides to change the immunostimulatory profile.

A pharmaceutical agent used in accordance with the present disclosure may comprise or consist of one or more cells. Therapeutic administration of cells may be used for stem, progenitor, or mature cell engraftment, differentiation, and long-term replacement of damaged tissue. In this paradigm multipotent or unipotent cells differentiate into a specific cell type in the lab or after reaching the site of injury (via local or systemic administration). These cells then integrate into the site of injury, replacing damaged tissue, and thus facilitate improved function of the organ or tissue. An example of this is the use of cells to replace cardiomyocytes after myocardial infarction, to facilitate angiogenesis in ischemic limb disease, or to produce cartilage in intervertebral disc degeneration. Further therapeutic uses of cells include administration of cells that have the capacity to release soluble factors such as cytokines, chemokines, and growth factors which act in a paracrine or endocrine manner. Examples of this include cells that secrete factors which facilitate angiogenesis, anti-inflammation, and anti-apoptosis. Adherent stromal cells or mature endothelial cells may be used to treat peripheral artery disease and arteriovenous access complications. Cells administered in accordance with the present disclosure may be allogenic, autologous or xenogenic. Examples of suitable cell types include Human embryonic stem cells, Neural stem cells, Mesenchymal stem cells, Hematopoietic stem cells, and Differentiated or mature cells.

A pharmaceutical agent used in accordance with the present disclosure may comprise or consist of a therapeutic microorganism such as a bacterium. Examples include the use of tumor homing bacteria that are able to target cancerous cells in the body. Some common tumor homing bacteria include Salmonella, Clostridium, Bifidobacterium, Listeria, and Streptococcus. Salmonella bacteria typically kill tumour cells by uncontrolled bacterial multiplication that can lead to the bursting of cancerous cells, and Salmonella-colonized tumours typically secrete anti-tumour IL-1β. S. Typhimurium flagellin is believed to increase both innate and adaptive immunity (nonspecific and specific defence mechanisms) of the bacteria by stimulating NK cells (Natural Killer cells) to produce interferon-γ (IFN-γ), an important cytokine (regulatory protein) for this immunity. Listeria is believed to inhibit tumors through NADPH oxidase mediated production (nicotinamide adenine dinucleotide phosphate oxidase) of ROS (reactive oxygen species) which is a cell signaling process that activates CD8+ T cells (cells that kill cancerous tissue) which target primary tumors. Furthermore, Clostridium, S. Typhimurium, and Listeria are believed to produce exotoxins (e.g. phospholipases, hemolysins, lipases) that damage the membrane structure and the cellular functions of the tumor using apoptosis or autophagy which is programmed cell death. Salmonella, Clostridium, and Listeria infections may also promote tumor elimination by increasing cytokines and chemokines that regulate infected sites. Mycobacterium Bovis, also known as Bacillus Calmette-Guérin (BCG), is a confirmed treatment for bladder cancer.

An exemplary pharmaceutical agent used in accordance with the present disclosure is R21. R21 is a novel pre-erythrocytic candidate malaria vaccine which includes HBsAg fused to the C-terminus and central repeats (Asn-Ala-Asn-Pro [NANP]) of the CSP (circumsporozoite protein), which self-assemble into virus-like particles in yeast. R21 lacks the excess HBsAg found in other malaria vaccine candidates such as RTS,S. R21 comprises only fusion protein moieties, in contrast to RTS,S, which comprises 20% with the remaining 80% being HBsAg monomers expressed alone, thereby likely diminishing CSP coverage of the virus-like particle surface. The R21 pre-erythrocytic malaria vaccine candidate was developed at the University of Oxford (Oxford, UK) and is currently manufactured by the Serum Institute of India (Pune, India).

Sometimes, the particles of the present disclosure and/or a composition comprising such particles comprises an adjuvant. When an adjuvant is present it may be present in the core of the particles (e.g. it may be present in the aqueous phase in the disclosed methods) and/or it may be present in a pharmaceutical composition comprising the particles and one or more further components. An adjuvant may be present in the core of the particles and in a pharmaceutical composition comprising the particles. A first adjuvant may be comprised in the core of the particles and a second adjuvant may be comprised in a pharmaceutical composition comprising the particles. As will be apparent to those skilled in the art, when an adjuvant is present in the core of a particle then it will have been present in the aqueous phase discussed above.

The term “adjuvant” as used herein refers to a substance which is used to enhance immunomodulation. Examples of adjuvants include: aluminum salts such as alum, aluminum hydroxide or aluminum phosphate, calcium salts (e.g. calcium phosphate hydroxide), iron salts, zinc salts, an insoluble suspension of acylated tyrosine, acylated sugars, cationically or anionically derivatised saccharides, polyphosphazenes, biodegradable microspheres, lipid nanoparticles, monophosphoryl lipid A (MPL), lipopolysaccharides, lipid A derivatives (e.g. of reduced toxicity), 3-O-deacylated MPL [3D-MPL], detergents, e.g. quil A, Saponin, QS21, Freund's Complete Adjuvant, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, oils, e.g. squalene, mineral oils e.g. paraffin oil, food-based oils e.g. adjuvant 65 (derived from peanut oil), bacterial products, e.g. killed bacteria selected from Bordatella pertussis, Mycobacterium bovis, toxoids, bioadhesives and mucoadhesives, microparticles, liposomes, polyoxyethylene ether formulations, polyoxyethylene ester formulations, muramyl peptides or imidazoquinolone compounds (e.g. imiquamod and its homologues), human immunomodulators suitable for use as adjuvants including cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte and macrophage colony stimulating factor (GM-CSF). An exemplary adjuvant used herein is a liposome-based adjuvant with saponin QS21 and a TLR-4 agonist integrated into the liposomal bilayer, which is suitable for use with the R21 vaccine composition described in Example 9.

Sometimes, the particles of the present disclosure and/or a composition comprising such particles comprises a cytokine. When a cytokine is present it may be present in the core of the particles (e.g. it may be present in the aqueous phase in the disclosed methods) and/or it may be present in a pharmaceutical composition comprising the particles and one or more further components. A cytokine may be present in the core of the particles and in a pharmaceutical composition comprising the particles. A first cytokine may be comprised in the core of the particles and a second cytokine may be comprised in a pharmaceutical composition comprising the particles. As will be apparent to those skilled in the art, when a cytokine is present in the core of a particle then it will have been present in the aqueous phase discussed above. Cytokines are particularly useful in embodiments of the present disclosure wherein the disclosed particles and compositions comprising them are used in the context of vaccinations, as cytokines may enhance the immunogenicity of the particles or compositions.

A cytokine may be selected from the group consisting of a transforming growth factor (TGF) such as but not limited to TGF-α and TGF-β; insulin-like growth factor-I and/or insulin-like growth factor-II; erythropoietin (EPO); an osteoinductive factor; an interferon such as but not limited to interferon-.α, -β, and -γ; a colony stimulating factor (CSF) such as but not limited to macrophage-CSF (M-CSF); granulocyte-macrophage-CSF (GM-CSF); and granulocyte-CSF (G-CSF). In some embodiments, the cytokine is selected from the group consisting of nerve growth factors such as NGF-β; platelet-growth factor; a transforming growth factor (TGF) such as but not limited to TGF-α, and TGF-β; insulin-like growth factor-I and insulin-like growth factor-II; erythropoietin (EPO); an osteoinductive factor; an interferon (IFN) such as but not limited to IFN-α, IFN-β, and IFN-γ; a colony stimulating factor (CSF) such as macrophage-CSF (M-CSF); granulocyte-macrophage-CSF (GM-CSF); and granulocyte-CSF (G-CSF); an interleukin (II) such as but not limited to IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18; LIF; kit-ligand or FLT-3; angiostatin; thrombospondin; endostatin; a tumor necrosis factor (TNF); and LT.

Typically, an adjuvant or cytokine can be added in an amount of about 0.01 mg to about 10 mg per dose, preferably in an amount of about 0.2 mg to about 5 mg per dose. Alternatively, the adjuvant or cytokine may be at a concentration of about 0.01 to 50%, preferably at a concentration of about 2% to 30%.

Further therapeutic agents which may be comprised in the particles of the present disclosure and/or in a composition comprising such particles; or which may be administered in combination with the particles of the present disclosure and/or in a composition comprising such particles, include: checkpoint blockade therapy/checkpoint inhibitors, co-stimulatory antibodies, cytotoxic or non-cytotoxic chemotherapy and/or radiotherapy, targeted therapy or monoclonal antibody therapy. It has been demonstrated that chemotherapy sensitizes tumors to be killed by tumor specific cytotoxic T cells induced by vaccination (Ramakrishnan et al. J Clin Invest. 2010; 120 (4): 1111-1124). Examples of chemotherapy agents include alkylating agents including nitrogen mustards such as mechlorethamine (HN2), cyclophosphamide, ifosfamide, melphalan (L-sarcolysin) and chlorambucil; anthracyclines; epothilones; nitrosoureas such as carmustine (BCNU), lomustine (CCNU), semustine (methyl-CCNU) and streptozocin (streptozotocin); triazenes such as decarbazine (DTIC; dimethyltriazenoimidazole-carboxamide; ethylenimines/methylmelamines such as hexamethylmelamine, thiotepa; alkyl sulfonates such as busulfan; Antimetabolites including folic acid analogues such as methotrexate (amethopterin); alkylating agents, antimetabolites, pyrimidine analogs such as fluorouracil (5-fluorouracil; 5-FU), floxuridine (fluorodeoxyuridine; FUdR) and cytarabine (cytosine arabinoside); purine analogues and related inhibitors such as mercaptopurine (6-mercaptopurine; 6-MP), thioguanine (6-thioguanine; TG) and pentostatin (2′-deoxycoformycin); epipodophylotoxins; enzymes such as L-asparaginase; biological response modifiers such as IFNα, IL-2, G-CSF and GM-CSF; platinum coordination complexes such as cisplatin (cis-DDP), oxaliplatin and carboplatin; anthracenediones such as mitoxantrone and anthracycline; substituted urea such as hydroxyurea; methylhydrazine derivatives including procarbazine (N-methylhydrazine, MIH) and procarbazine; adrenocortical suppressants such as mitotane (o.p′-DDD) and aminoglutethimide; taxol and analogues/derivatives; hormones/hormonal therapy and agonists/antagonists including adrenocorticosteroid antagonists such as prednisone and equivalents, dexamethasone and aminoglutethimide, progestin such as hydroxyprogesterone caproate, medroxyprogesterone acetate and megestrol acetate, estrogen such as diethylstilbestrol and ethinyl estradiol equivalents, antiestrogen such as tamoxifen, androgens including testosterone propionate and fluoxymesterone/equivalents, antiandrogens such as flutamide, gonadotropin-releasing hormone analogs and leuprolide and non-steroidal antiandrogens such as flutamide; natural products including vinca alkaloids such as vinblastine (VLB) and vincristine, epipodophyllotoxins such as etoposide and teniposide, antibiotics such as dactinomycin (actinomycin D), daunorubicin (daunomycin; rubidomycin), doxorubicin, bleomycin, plicamycin (mithramycin) and mitomycin (mitomycin C), enzymes such as L-asparaginase, and biological response modifiers such as interferon alphenomes.

### Compositions

The particles of the present disclosure may be provided in the form of a pharmaceutical composition, the pharmaceutical composition comprising a particle of the present disclosure and one or more pharmaceutically acceptable excipient, diluent or adjuvant.

Preferred pharmaceutical compositions are sterile and pyrogen free.

When the pharmaceutical compositions provided herein comprise one or more optically active components (e.g. one or more optically active pharmaceutical agents), such components (e.g. the pharmaceutical agents) are typically in the form of a substantially pure optical isomer. For the avoidance of doubt, said components may be administered in the form of a solvate.

Typically, the composition contains up to 85 wt % of the particles of the disclosure. More typically, it contains up to 50 wt % of the particles of the disclosure, for example up to 40 wt %, up to 30 wt %, up to 20 wt %, or up to 10 wt % of the particles of the disclosure.

In some embodiments the composition comprises a physiologically acceptable medium in which the particles may be administered to a subject. The medium may comprise or consist of water (e.g. deionised and/or ultrapure water). The medium may have a pH of from about 4 to about 10. More usually the medium has a pH of from about 5 to about 9, such as from about 6 to about 8. The medium may comprise one or more buffer salts. Buffer salts are often present with one or more additional pharmaceutically acceptable salts in order that the resulting composition is isotonic e.g. with human blood plasma. Pharmaceutically acceptable buffers include phosphate/phosphoric acid, citrate/citric acid, acetate/acetic acid, histidine, lactate/lactic acid, trometamine, gluconic acid/gluconate, aspartic acid/aspartate, tartaric acid/tartarate, succinic acid/succinate, malic acid/malate, fumaric acid/fumarate and α-ketoglutaric acid/α-ketoglutarate. Most often the buffer salt is selected from phosphate, citrate and acetate. A preferred medium is phosphate buffered saline (PBS). In some embodiments PBS comprises NaCl and/or KCl, buffered to about 7.4 using phosphate buffer. For example, PBS may comprise 137 mM NaCl, 2.7 mM KCl, 8 mM Na₂HPO₄, and 2 mM KH₂PO₄.

As explained above, the particles of the present disclosure may be used to deliver any suitable therapeutic agent. As will be apparent, one or more therapeutic agents are typically comprised in the aqueous core of the particles disclosed herein. When formulated into a composition comprising further components in addition to said particles, one or more further pharmaceutical agents may be comprised in the medium in which the particles are present. Any of the pharmaceutical agents disclosed herein may be used in either the aqueous core of the particles and/or the medium of a composition comprising such particles.

The particles of the present disclosure and compositions comprising them may be useful for delivery of vaccines. Therefore provided herein is a vaccine composition comprising a plurality of particles as disclosed herein, wherein said particles comprises a pharmaceutical agent which is a first immunogenic agent.

The delayed release nature of the particles means that they are suitable for use in prime/boost vaccinations.

In this context those skilled in the art will appreciate that, as discussed above, the goal of vaccination is to elicit long-lasting immune memory, in order to mediate protection from infection, or at least to prevent disease in case of exposure to the pathogen. Multiple immunizations are required for most vaccine strategies, to induce efficient protection.

Many vaccines require multiple doses to induce long-lasting protective immunity in a high frequency of vaccines, and to ensure strong both individual and herd immunity. Repetitive immunogenic stimulations not only increase the intensity and durability of adaptive immunity, but also influence its quality.

The terms “primary doses,” may be used interchangeably with “boosts,” particularly when multiple vaccine injections are close in time to each other. Typically, a very first vaccine dose activates naïve T and B cells, which undergo proliferation, contraction and a differentiation program to develop into primary memory T cells. As soon as the second vaccine dose is administered, when the primary effector response has started to contract, it can be called a boost. Repeated administrations using the very same vaccine, which are called homologous prime/boost, have proven to be very effective for augmenting humoral responses. However, they appeared to be relatively less efficient at enhancing cellular immunity, likely because prior immunity to the vaccine tends to impair robust Ag presentation and the generation of appropriate inflammatory signals for T cells. In contrast, the sequential administration of vaccines using different Ag delivery systems is called heterologous prime/boost. It has proven to be effective at generating high levels of memory T cells in preclinical studies and clinical trials.

Several vaccine parameters are known to influence adaptive immune responses, including notably the number of immunizations, the delay between them, and the delivery sequence of different recombinant vaccine vectors. The delayed release nature of the particles of the present disclosure means that compositions comprising such particles can be ideally used to deliver prime/boost benefits in a single administration, with the prime dose arising from a therapeutic agent in the bulk composition and the boost arising from a therapeutic agent (either the same or different) in the aqueous core of the particles.

Accordingly, further provided therefore is a prime/boost vaccine composition comprising:
(i) a plurality of particles as disclosed herein, wherein said particles comprises a pharmaceutical agent which is a first immunogenic agent; and
(ii) a second immunogenic agent;
   wherein the first immunogenic agent and the second immunogenic agent are the same or different. When the two immunogenic agents are the same the prime/boost vaccination composition may be referred to as homologous prime/boost vaccination composition. When the two immunogenic agents are different the prime/boost vaccination composition may be referred to as heterologous prime/boost vaccination composition.

The particles of the disclosure and compositions comprising such particles may be administered in a variety of dosage forms. Thus, they can be administered orally, for example as tablets, troches, lozenges, aqueous or oily suspensions, dispersible powders or granules. They may also be administered parenterally, whether subcutaneously, intravenously, intramuscularly, intrasternally, transdermally or by infusion techniques. The compound, composition or combination may also be administered as a suppository. Preferably, the compound, composition or combination is administered via parenteral administration, in particular via intravenous administration.

The particles of the disclosure and compositions comprising such particles are typically formulated for administration with a pharmaceutically acceptable carrier or diluent.

For example, solid oral forms may contain diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

The particles of the disclosure and compositions comprising such particles may be formulated for pulmonary administration. For example, the particles of the disclosure and compositions comprising such particles may be formulated for inhaled (aerosolised) administration as a solution or suspension. The particles of the disclosure and compositions comprising such particles may be administered by a metered dose inhaler (MDI) or a nebulizer such as an electronic or jet nebulizer. Alternatively, the particles of the disclosure and compositions comprising such particles may be formulated for inhaled administration as a powdered drug; such formulations may be administered from a dry powder inhaler (DPI).

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. Suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection may contain as carrier, for example, sterile water or preferably they may be in the form of sterile, aqueous, isotonic saline solutions. Compositions for injection (e.g. i.v. administration) may contain excipients for increasing the solubility of component compounds. Suitable excipients include cyclodextrins such as captisol. Pharmaceutical compositions suitable for delivery by needleless injection, for example, transdermally, may also be used.

### Therapeutic Efficacy

The particles of the disclosure and compositions comprising such particles are particularly advantageous in the medical setting.

The particles of the disclosure and compositions comprising such particles are therapeutically useful. Accordingly, provided herein are particles of the disclosure and compositions comprising such particles for use in medicine. The present invention further provides particles of the disclosure and compositions comprising such particles, for use in treating the human or animal body. The present invention further provides particles of the disclosure and compositions comprising such particles, for use in preventing disease in the human or animal body.

The particles of the disclosure and compositions comprising such particles may be administered in combination with one or more further pharmaceutically active agents as described herein. The active agents may each be provided in a single formulation or one or more of them may be separately formulated. Where separately formulated, the two or more agents may be administered simultaneously or separately. Active agents which are separately formulated may be provided in the form of a kit, optionally together with instructions for their administration.

In some cases the particles of the disclosure and compositions comprising such particles are used in a method of vaccination or a method of providing immunotherapy. As used herein, “immunotherapy” is the treatment or prevention of a disease or condition by inducing or enhancing an immune response in an individual. In certain embodiments, immunotherapy refers to a therapy that comprises the administration of one or more drugs to an individual to elicit T cell responses. T cell responses may be cytotoxic T cell response against cells that display tumor associated antigens (TAAs), tumor specific antigens (TSAs) or cancer testis antigens (CTAs) on their cell surface. In another embodiment, immunotherapy refers to a therapy that comprises the administration or expression of polypeptides to an individual to elicit a T helper response to provide co-stimulation to cytotoxic T cells that recognize and kill diseased cells. In still another embodiment, immunotherapy refers to a therapy that comprises administration of one or more drugs to an individual that re-activate existing T cells to kill target cells.

In some instances, immunotherapy may be used to treat tumours. In other instances, immunotherapy may be used to treat intracellular pathogen-based diseases or disorders. In some cases the disclosure relates to the treatment of cancer or any specific type of cancer described herein. In some other cases the disclosure relates to the treatment of a viral, bacterial, fungal or parasitic infection, or any other disease or condition that may be treated by immunotherapy.

The particles of the present disclosure may be used to deliver vaccine therapies to address (e.g. to prevent or treat, typically to prevent) any suitable disorder. Exemplary disorders that can be addressed by vaccines include cholera, COVID-19, dengue fever, diphtheria, ebola, Haemophilus influenzae type b, hepatitis A, hepatitis B, hepatitis E, human papillomavirus infection, influenza, Japanese encephalitis, malaria, measles, meningococcal disease, mumps, pneumococcal disease, pertussis, poliomyelitis, rabies, rotavirus gastroenteritis, rubella, tetanus, tick-borne encephalitis, tuberculosis, typhoid fever, varicella, yellow fever, shingles (Herpes Zoster), Bordetella, canine distemper, canine influenza, canine parvovirus, chlamydia, feline calicivirus, feline distemper, feline leukemia, feline viral rhinotracheitis, leptospirosis and lyme disease. In some preferred embodiments the particles of the present disclosure may be used to deliver a vaccine for the prevention or treatment of malaria.

Accordingly, provided herein are particles of the disclosure and compositions comprising such particles for use in treating or preventing a disease in a subject in need thereof, wherein said use comprises administering said particles or said composition to said subject, thereby inducing an immunogenic response in said subject. Also provided is a method of inducing an immunogenic response in a subject in need thereof, said method comprising administering to said subject a therapeutically effective amount of one or more particles of the disclosure and/or one or more compositions comprising such particles. Further provided are the use of particles of the disclosure and compositions comprising such particles in the manufacture of a medicament for inducing an immunogenic response in a subject. In some embodiments said particles and compositions are for use in treating or preventing a disorder described herein. In some embodiments said particles and compositions are for use in treating or preventing a disorder described herein, wherein said use comprises administering said particles and compositions as a prime/boost vaccine. In some embodiments said particles and compositions are for use in preventing a disorder described herein, wherein said use comprises administering said particles and compositions as a prime/boost vaccine. In some embodiments said disorder is malaria.

Further provided are particles of the disclosure and compositions comprising such particles for use in a method of vaccination, said use comprising administering to a subject in need thereof an effective amount of said particles and/or said composition. Also provided is a method of vaccination, said method comprising administering to a subject in need thereof an effective amount of one or more particles of the disclosure and/or compositions comprising such particles. Further provided is the use of particles of the disclosure and/or compositions comprising such particles in the manufacture of a vaccine or in the manufacture of a medicament to be administered as a vaccine. In some embodiments an effective amount is a therapeutically effective amount of said particles and/or said composition to treat a pathological condition such as a condition disclosed herein. In some embodiments an effective amount is a prophylactically effective amount of said particles and/or said composition to prevent a pathological condition such as a condition disclosed herein.

As explained above, compositions comprising the particles of the present disclosure are ideal for use in prime/boost vaccinations. Accordingly, further provided are compositions comprising (i) particles of the present disclosure, wherein said particles comprise a first immunogenic agent; and (ii) a second immunogenic agent; for use in a method of prime/boost vaccination, said use comprising administering to a subject in need thereof an effective amount of said composition. In some embodiments said particles comprise a therapeutic agent suitable for use as a vaccine as provided herein, such as a therapeutic agent suitable for use as a vaccine for treating or preventing (typically preventing) malaria. In some embodiments said therapeutic agent comprises a vaccine antigen. In some embodiments the therapeutic agent comprises a pre-erythrocytic malaria vaccine.

Also provided is a method of prime/boost vaccination, said method comprising administering to a subject in need thereof an effective amount of a composition comprising (i) particles of the present disclosure, wherein said particles comprise a first immunogenic agent; and (ii) a second immunogenic agent.

Further provided is the use of a composition comprising (i) particles of the present disclosure, wherein said particles comprise a first immunogenic agent; and (ii) a second immunogenic agent in the manufacture of a medicament to be administered as a prime/boost vaccine. In such uses and methods the first and second immunogenic agents may be the same or different. When the two immunogenic agents are the same the prime/boost vaccination method may be referred to as homologous prime/boost vaccination. When the two immunogenic agents are different the prime/boost vaccination method may be referred to as heterologous prime/boost vaccination.

Those skilled in the art will appreciate that the term “prime/boost” as used herein does not necessarily require that the particles provided herein are administered multiple times, although this is not excluded. Rather, the delayed release properties of particles provided herein means that a prime/boost regimen can be provided by administering a single composition comprising both an initial therapeutic agent and particles of the present invention comprising a secondary therapeutic agent, which may be the same or different to the initial therapeutic agent. The initial therapeutic agent provides an initial therapeutic effect (“prime”) and the secondary therapeutic agent following release from the particles provides the secondary therapeutic effect (“boost”). For example, a therapeutic agent such as a vaccine agent can be provided in an aqueous suspension and a further vaccine agent which may be the same or different can be provided in particles suspended in the aqueous suspension. Such a composition and its therapeutic uses is described in Example 9 and constitutes a single-dose prime/boost strategy for effective therapy. Example 9 describes an exemplary embodiment using R21 vaccine against malaria as but the invention is not limited accordingly and those skilled in the art will appreciate that other vaccines could be used for the same or other disorders, according to the requirements of the user of the invention.

Further provided are particles of the disclosure and compositions comprising such particles for use in a method of immunotherapy, said use comprising administering to a subject in need thereof an effective amount of said particles and/or said composition. Also provided is a method of immunotherapy, said method comprising administering to a subject in need thereof an effective amount of one or more particles of the disclosure and/or compositions comprising such particles. Further provided is the use of particles of the disclosure and/or compositions comprising such particles in the manufacture of an immunotherapy medicament. In some embodiments an effective amount is a therapeutically effective amount of said particles and/or said composition to treat a pathological condition such as a condition disclosed herein. In some embodiments an effective amount is a prophylactically effective amount of said particles and/or said composition to prevent a pathological condition such as a condition disclosed herein.

In one aspect, the subject to be treated is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

The disclosed particles and/or compositions may be administered to the subject in order to prevent the onset or reoccurrence of one or more symptoms of a pathological condition. This is prophylaxis. In this embodiment, the subject can be asymptomatic. A prophylactically effective amount of the particles and/or compositions is administered to such a subject. A prophylactically effective amount is an amount which prevents the onset of one or more symptoms of the pathological condition.

The disclosed particles and/or compositions may be administered to the subject in order to treat one or more symptoms of a pathological condition. In this embodiment, the subject is typically symptomatic. A therapeutically effective amount of the particles and/or compositions is administered to such a subject. A therapeutically effective amount is an amount effective to ameliorate one or more symptoms of the disorder.

A therapeutically or prophylactically effective amount of the compound of the invention is administered to a subject. The dose may be determined according to various parameters, especially according to the compound used; the age, weight and condition of the subject to be treated; the route of administration; and the required regimen. A physician will be able to determine the required route of administration and dosage for any particular subject. A typical daily dose is from about 0.01 to 100 mg per kg, preferably from about 0.1 mg/kg to 50 mg/kg. e.g. from about 1 to 10 mg/kg of body weight, according to the activity of the specific agent, the age, weight and conditions of the subject to be treated, the type and severity of the disease and the frequency and route of administration. Preferably, daily dosage levels are from 70 mg to 3.5 g. Sometimes, higher dosages are required, such as from about 0.01 to about 250 mg per kg. e.g. from about 0.1 mg/kg to about 200 mg/kg, e.g. from about 1 to about 150 mg/kg of body weight, according to the activity of the specific agent, the age, weight and conditions of the subject to be treated, the type and severity of the disease and the frequency and route of administration. Such preferred daily dosage levels may be e.g. from 70 mg to 8 g. Higher dosages may be particularly suitable if the agent is administered to the subject multiple times per day, such as 2, 3 or 4 times per day, e.g. 4 times daily. A suitable daily dosage may be from 70 mg to 8 g per day administered in 2, 3 or 4 separate dosages.

### Further Aspects of the Disclosure

Also provided herein is an aqueous-core polymeric-shell particle obtainable by carrying out a method disclosed herein. In some embodiments the particle is a particle as described in more detail herein; e.g. the particle may comprise an aqueous core as described herein; may comprise a polymeric shell as described herein; may be comprised in a composition as described herein; and/or may be for use in a medicine as described in more detail herein.

Also provided herein is a population of aqueous-core polymeric-shell particles. The particles in the population are characterised inter alia by their homogeneity. In some embodiments the disclosure thus provides a homogeneous population comprising a plurality of aqueous-core polymeric-shell particles; wherein at least 90%, at least 95%, at least 96%, at least 97%, at least 98%, at least 99%, at least 99.5%, at least 99.9% or at least 99.99% of the particles in the population, preferably at least 99.999% or at least 99.9999% of the particles in the population are characterised as comprising:
a single, spherical, aqueous core volume having a mean diameter of from about 1 μm to about 300 μm (e.g. from about 3 μm to about 150 μm, e.g. from about 10 μm to about 100 μm, such as from about 30 μm to about 70 μm e.g. from about 40 μm to about 55 μm) and wherein the smallest diameter of the aqueous core is at least 70% (e.g. at least 90%, at least 95%, at least 96%, at least 97%, at least 98%, at least 99%, at least 99.9%, at least 99.99% or at least 99.999%) of the largest diameter of the aqueous core; and
a biodegradable polymeric shell having a thickness of from about 0.1 μm to about 100 μm (e.g. from about 1 μm to about 50 μm, such as from about 3 μm to about 20 μm e.g. from about 5 to about 15 μm, such as about 10 μm) and wherein the thickness of the thinnest part of the polymeric shell is at least 70% (e.g. at least 90%, at least 95%, at least 96%, at least 97%, at least 98%, at least 99%, at least 99.9%, at least 99.99% or at least 99.999%) of the thickness of the thickest part of the polymeric shell.

In some embodiments the particles in the population each comprise a buffered aqueous core comprising one or more pharmaceutical agents as defined herein; and/or the biodegradable polymer in the shell comprises poly(lactic-co-glycolic acid) (PLGA) having an average molecular weight of from about 1 kDa to about 250 kDa (e.g. from about 5 kDa to about 100 kDa e.g. from about 10 kDa to about 50 kDa) and comprising a ratio of lactic acid to glycolic acid (L:G ratio) of from 60:40 to 90:10 (e.g. from about 70:30 to about 80:20 e.g. about 75:25.

As explained above, previously known methods are typically not capable of providing such homogeneous populations as the methods used to produce known particles are not capable of such high degrees of precision and uniformity. Without being bound by theory, the inventors believe that the improvements in the homogencity of populations of particles achievable in accordance with the present disclosure may lead to beneficial effects in terms of providing for precisely controllable release of a pharmaceutical agent from the population of particles e.g. when administered in vivo as described herein. Accordingly, the population of particles as provided herein may be used in a therapeutic method as described herein, such as in the treatment or prevention of one or more pathological conditions as described herein.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

This example describes the production of particles in accordance with the present disclosure.

Particles shown in FIG. 8 were produced using a microfluidic chip design as described herein. The aqueous, solvent and aqueous extraction phases inlet widths were respectively 20, 40 and 80 μm. The heights of the channels are 50 μm until the aqueous extraction intersection and 100 μm after. The design was made using a CAD software (AutoCAD, Autodesk) and a positive mould was created from this design using soft lithography techniques provided by an external supplier (Micrux technologies). Briefly, a negative photoresist was spin coated on a silicon wafer, baked and selectively exposed to UV using a mask containing the corresponding design of the chip. The wafer was then developed by chemical treatment to keep the positive structures of the chip on it. After further baking, the mould was silanized with trimethylchlorosilane, to reduce the stickiness of polydimethylsiloxane (PDMS).

Once produced, a 10:1 weight mix of PDMS prepolymer and curing agent (Sylgard 184) was poured onto the positive mould, and the PDMS was cured overnight at 60° C. in an oven, before being peeled off the mould, resulting in the design being engraved inside the PDMS block (negative print). Inlets and outlets were punched using a 18G blunt needle (BD) and the chip was sealed with another slab of PDMS using 02 Plasma cleaner treatment (HPT-200, Henniker) as a surface activation. Immediately after bonding, a coating solution of 1% of polyvinyl alcohol (PVA) (87-90% hydrolysed, 30-70 kDa molecular weight-MW) was injected through the extraction solvent inlet to coat the second intersection of the design and downstream, spatially restrained by injection of air from the solvent and aqueous core inlets. The coating solution was flowing for 5 min, the chip was then dried with air and cured on a hot plate at 115° C. This cycle was repeated 3 times to ensure successful hydrophilic coating in the correct channels. The chip was then kept in an oven at 60° C. for 2 days before being used for microfluidic production.

The microfluidic production resulting in particles shown in FIG. 8 was conducted using chips manufactured as described above. Flows were injected in the chip using a pressure controller (OB1 controller, Elveflow) and the production was monitored using an inverted microscope (DMi8, Leica) and a high-speed camera (Nac MEMRECAM). The aqueous core, solvent and extraction flows were injected at initial pressures of 50 mbar, 120 mbar and 280 mbar respectively and then adjusted visually to achieve a correct throughput and size of emulsification. The composition of the flows was the following:
Inner Aqueous Core: 50 mg/mL Dextran TRITC (40 kDa MW) in PBS (pH 7.4, 1X)
Solvent with Polymer: 17.5% w/v PLGA (7-17 kDa MW. 50:50 L:G) in DMC.
Aqueous Extraction Phase: 3% w/v PVA (9-10 kDA. 80% hydrolysed) in DPBS
   (TRICT=tetramethylrhodamine; PBS=phosphate buffered saline; DMC=dimethyl carbonate; DPBS=Dulbecco's phosphate buffered saline)

The resulting double emulsions were collected in 10 mL of aqueous extraction phase in a glass petri dish (FIG. 8, left). The shells were hardened by extracting the solvent in large volumes of aqueous extraction phase in glass vials, rotating at room temperature for 24 hrs, resulting in a solid PLGA shell, liquid dextran loaded aqueous cores particles (FIG. 8, right). Images were taken with an inverted fluorescent microscope (Eclipse TiE, Nikon) overlaying the brightfield image with the signal from TRITC imaging.

The resulting particle production displayed a high monodispersity and regularity in their core-shell geometry suitable for medical applications such as the delayed delivery of pharmaceutical ingredient.

### Example 2

This example demonstrates that the release profile of particles produced in accordance with the present disclosure can be controlled by controlling the polymer used to encapsulate the aqueous core.

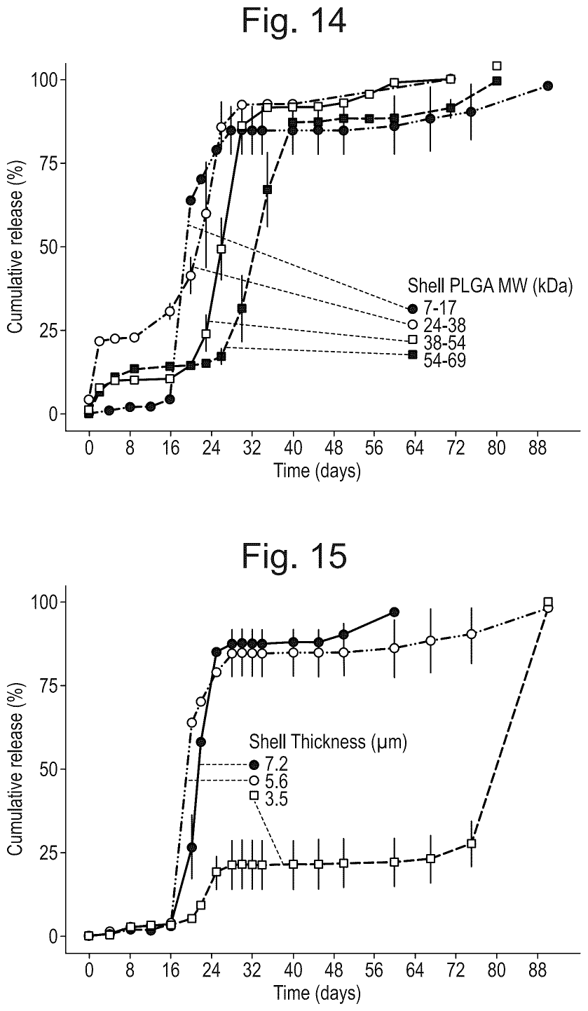

Particles with polymeric shells of different molecular weight (MW) were produced as described in Example 1, only adjusting the flow composition of the solvent phase to change the MW of the polymer. The following compositions of solvent phase were used:
17.5% w/v PLGA (7-17 kDa MW. 50:50 L:G) in DMC (FIG. 14, data in red in original colour image),
12.5% w/v PLGA (24-38 kDa MW. 50:50 L:G) in DMC (FIG. 14, data in blue in original colour image).
10.5% w/v PLGA (38-54 kDa MW, 50:50 L:G) in DMC (FIG. 14, data in yellow in original colour image),
8.5% w/v PLGA (54-69 kDa MW, 50:50 L:G) in DMC (FIG. 14, data in green in original colour image).
   while the other compositions of aqueous core and extraction phase remained unchanged. Batches of about 100 000 particles were made for each formulation (one batch per MW).

An in vitro model was used to evaluate the release profile of these different types of particle batches. Thus, for each MW formulation, particles were divided in 3 replicates of equal amount of particles and suspended in 2 mL of PBS (pH 7.4, 1X) in LoBind Eppendorf tubes. The tubes were incubated at 37° C. protected from light, at constant rotation of 20 rpm.

At regular time intervals particles were sedimented gently by centrifugation (30 rcf. 1 min) and 1 mL of supernatant was collected to measure the amount of dextran-TRITC released, using spectrometry (520/560 nm ex/em, Fluostar Omega, BMG Labtech). The supernatant was replenished with fresh PBS. Release curves were made by normalising the released amount of dextran-TRITC from the aqueous cores at a given time point against the total amount encapsulated originally. The resulting cumulative curves for each formulation are shown in FIG. 14 (mean of the 3 replicates, and error bar representing standard deviation).

As illustrated by FIG. 14, the in vitro delay of release from batches of particles is influenced by the MW of the polymer constituting the particles shells. A higher MW increases the delay before the release of the core content (dextran-TRITC in this example). Thus, increasing the MW from 7-17 kDa (FIG. 14, red) to 54-69 kDa (FIG. 14, green), results in an increase in the delay of about 2.5 weeks.

This example thus demonstrates that the release profile of particles produced in accordance with the present disclosure can be controlled by controlling the properties of the polymer used to encapsulate the aqueous core.

### Example 3

This example demonstrates that the release profile of particles produced in accordance with the present disclosure can be controlled by controlling the thickness of the polymeric shell used to encapsulate the aqueous core.

Particles with different shell thicknesses were produced as described in Example 1, only adjusting the injection pressure to control the relative sizes of the droplets. The solvent phase was identical to Example 1 (17.5% w/v PLGA 7-17 kDa MW, 50:50 L:G in DMC). The cores were loaded with dextran-TRITC (40 kDa MW) as the fluorescent payload model.

Injection pressures were adjusted from 40 to 50 mbar for the aqueous core phase and 100 to 120 mbar for the solvent phase to produce 3 particles batches of different shell thicknesses.

Shell thickness was measured by fluorescent imaging (36 images, Eclipse TiE. Nikon) and processed with a custom MATLAB script to measure the average shell thickness of each batch.

The release from each batch was evaluated in vitro following the protocol described in Example 2, with regular sampling of the released fluorescent dextran-TRITC.

As illustrated in FIG. 15, particles with 5.6 and 7.2 μm shell thickness (FIG. 15. red and orange in original colour image) released at the same time between 16 and 24 days, while thin shell particles of 3.5 μm shell thickness (FIG. 15, yellow in original colour image) displayed a small release around the same time but remained loaded for much longer. Without being bound by theory, the inventors believe that the difference may be due to a higher autocatalytic degradation occurring in particles with thicker shells, while thin shell ones do not keep the by-products of the degradation for long enough to reach high degrees of autocatalytic degradation and thus need longer times to release their payload.

This example thus demonstrates that the release profile of particles produced in accordance with the present disclosure can be controlled by controlling the thickness of the polymeric shell used to encapsulate the aqueous core.

### Example 4

This example demonstrates that the release profile of particles produced in accordance with the present disclosure in which the polymer is PLGA can be controlled by controlling the Lactide to Glycolide (L:G ratio) content of the PLGA polymer.

Particles with PLGA polymeric shells of different Lactide to Glycolide ratios (L:G) were produced as described in Example 1, only adjusting the flow composition of the solvent phase to change the L:G of the polymer. The following compositions of solvent phase were used:
12.5% w/v PLGA (24-38 kDA MW, 50:50 L:G) in DMC (FIG. 14, light blue).
12.5% w/v PLGA (24-38 kDA MW. 75:25 L:G) in DMC (FIG. 14, dark blue), while the other compositions of aqueous core and extraction phase remained unchanged. Batches of about 100 000 particles were made for each formulation (one batch per MW).

The release from each batch was evaluated in vitro following the protocol described in Example 2, with regular sampling of the fluorescent dextran-TRITC released.

As illustrated by FIG. 16, the in vitro delay of release from batches of particles is influenced by the L:G ratio of the polymer constituting the particles shells. A higher L:G ratio increases the delay before the release of the core content (dextran-TRITC in this example). Thus, increasing the L:G ratio from 50:50 (FIG. 14, light blue) to 75:25 (FIG. 14, dark blue) while keeping the MW constant at 24-38 kDA, results in a significant increase in the delay of about 8 weeks.

This example thus demonstrates that the release profile of particles produced in accordance with the present disclosure can be controlled by controlling the properties of the polymer used to encapsulate the aqueous core, e.g. by controlling the L:G ratio of a PLGA polymer.

### Example 5

This example demonstrates that the thickness of particles produced in accordance with the present disclosure can be controlled by controlling the injection pressures of the aqueous phase and solvent phase.

Particles with different shell thicknesses were produced as described in Example 1 and 3, by adjusting the injection pressures to control the relative sizes of the droplets. The solvent phase was identical to Example 1 (17.5% w/v PLGA 7-17 kDa MW, 50:50 L:G in DMC). The cores were loaded with dextran-TRITC (40 kDa MW) as the fluorescent payload model.

Injection pressures were adjusted to around 50 mbar and 100 mbar for the aqueous core phase and the solvent phase respectively (FIG. 17A.) or to 40 mbar and 120 mbar for the aqueous core phase and the solvent phase respectively (FIG. 17B). The extraction phase injection pressure was kept at 280 mbar. Small adjustments were made manually to the injection pressures as a result of visual monitoring using the high-speed camera.

As illustrated in FIG. 17, modifying the injection pressure ratios of the aqueous core phase and the solvent phase changes the thickness of the particles. By increasing the aqueous to solvent pressure ratio, thus increasing the aqueous to solvent flow rate ratios, particles with thinner shells (FIG. 17A) are produced. Conversely, particles with thicker shells (FIG. 17B) are produced with lower aqueous to solvent pressure ratios.

This example thus demonstrates that the release profile of particles produced in accordance with the present disclosure can be controlled by controlling the injection pressures, and thus the flow-rate ratios, of the aqueous phase and solvent phase in accordance with the methods of the present disclosure.

### Example 6

This example demonstrates that immune responses generated by in vivo administration of immunogenic agents formulated in particles in accordance with the present invention are increased by the robust nature of the particles.

Particles loaded with ovalbumin were produced as described in Example 1, adjusting the flows composition as follows:
Inner Aqueous Core: 20 mg/mL Ovalbumin (OVA) in PBS (pH 7.4, 1X)
Solvent with Polymer: 17.5% w/v PLGA (7-17 kDa MW. 50:50 L:G) in DMC
Aqueous Extraction Phase: 3% w/v PVA (9-10 kDA. 80% hydrolysed) in DPBS

The particles were solidified as described in Example 1. The amount of encapsulated OVA is measured by breaking a known amount of particles using a homogenizer and measuring the protein content by microBCA (ThermoFisher).

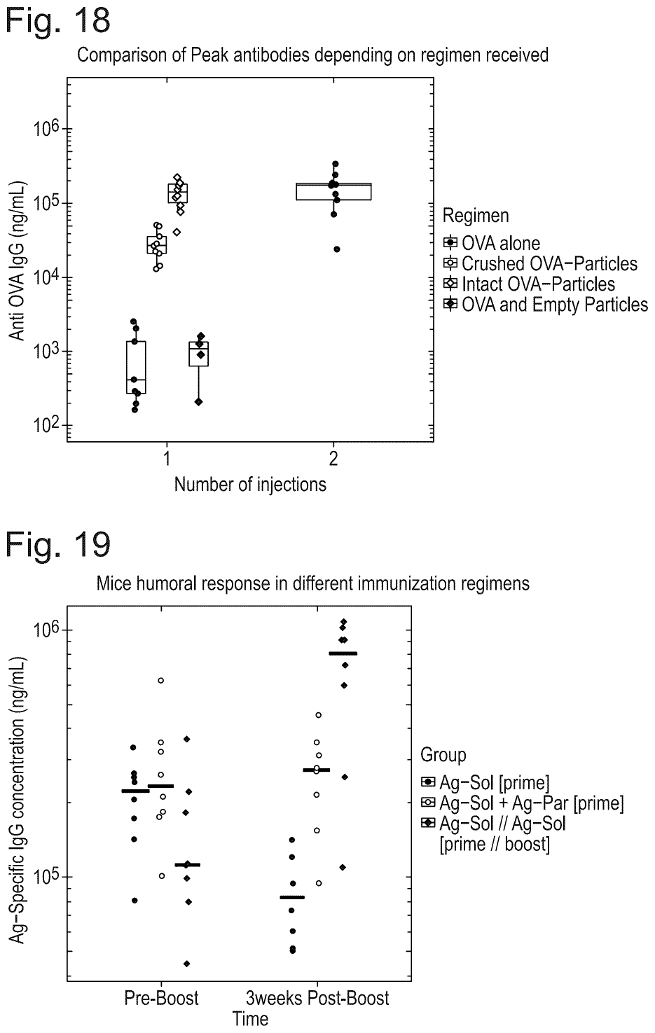

In vivo experiments were conducted in 4 groups of 10 female Balb/c mice, receiving doses of 30 μg of OVA per mouse in different regimens:
Group 1 (FIG. 18, green in original colour image):
   Prime OVA dose alone, booster OVA dose 23 weeks later.
Group 2 (FIG. 18, purple in original colour image):
   Prime OVA dose encapsulated in particles produced as described above and broken before injection.
Group 3 (FIG. 18, blue in original colour image):
   Prime OVA dose encapsulated in intact particles produced as described above.
Group 4 (FIG. 18, red in original colour image):
   Prime OVA dose admixed with empty PLGA capsules.

Weekly blood samples were taken from each mouse and OVA specific IgG antibody titers measured by ELISA. The peak antibody titre for each formulation is plotted in FIG. 18. As can be seen, an enhancement of the humoral immune response (OVA specific IgG antibody response) was observed with encapsulated OVA (in blue) due to the delayed release. During injection, due to the syringe pressure some particles are likely to have broken releasing a fraction of the vaccine dose, with the remaining capsules reclassing later. When comparing to OVA alone (in green) the level of antigen-specific antibody (Ab) titre from the intact OVA-particles is 100-fold higher and similar to a 2 injections regimen of OVA alone (blue to green comparison). The increase of Ab titers is not due to an adjuvant effect of the particles themselves as the regimen of OVA with empty particles (in red) was similar to OVA alone. Crushed OVA-particles (in purple) induced a higher response. Without being bound by theory, the inventors believe that this may be because PLGA debris could be internalised by immune cells enhancing the inflammation. Overall, a single immunisation with encapsulated OVA provides the highest Ab titer with the minimum number of injections.

### Example 7

This example describes the immunization of mice with malaria vaccines administered intramuscularly with R21 either in a soluble form or encapsulated inside particles according to the present disclosure.

To illustrate the ability of the particles to generate an immune response with a different antigen, R21 was used in this example. R21 is a virus like particle vaccine against malaria developed by the Jenner Institute in Oxford University.

Particles loaded with R21 were produced as described in Example 1, adjusting the flows composition as follows:
Inner Aqueous Core: 0.36 mg/mL R21 in PBS (pH 7.4, 1X)
Solvent with Polymer: 17.5% w/v PLGA (7-17 kDa MW, 50:50 L:G) in DMC
Aqueous Extraction Phase: 3% w/v PVA (9-10 kDA, 80% hydrolysed) in DPBS

The particles were solidified as described in Example 1. The amount of encapsulated R21 is measured by breaking a known amount of particles using a homogenizer and measuring the protein content by microBCA (ThermoFischer).

8 mice were immunized intramuscularly with lug of R21 (noted as Antigen-Ag) either in a soluble form (Ag-Sol) or encapsulated inside the particles (Ag-Par) produced as described above. As shown in FIG. 19, mice in blue (in original colour image) received a single prime injection of Ag-Sol, mice in green (in original colour image) received a double prime/boost injection regimen of Ag-Sol, and mice in orange (in original colour image) received a single prime injection of Ag-Sol and Ag-Par.

By three weeks after the booster in the relevant group, the Ab titers for the prime only Ag-Sol group (blue in original colour image) had decreased significantly while the mice receiving Ad-Par (orange in original colour image) maintained the same level of R21-specific Ab. This demonstrates the advantage of a prime-boost regimen achievable in accordance with the present invention.

Ab titres in the prime/boost regimen (green in original colour image) were higher; however, without being bound by theory, the inventors believe this may possibly be due to the presence of an adjuvant in the booster dose (no adjuvant was encapsulated in the particles).

### Example 8

This example demonstrates that the release profile of homogeneous particles produced in accordance with the present disclosure is improved relative to heterogeneous particles produced by conventional methods (batch emulsification by vortexing), with both improved delay and sharper release being observed.

Homogeneous particles were produced using microfluidics as described in Example 1, with the following flow phase compositions:
Inner Aqueous Core: 50 mg/mL Dextran TRITC (40 kDa MW) in PBS (pH 7.4, 1X)
Solvent with Polymer: 17.5% w/v PLGA (7-17 kDa MW, 50:50 L:G) in DMC.
Aqueous Extraction Phase: 3% w/v PVA (9-10 kDA. 80% hydrolysed) in DPBS

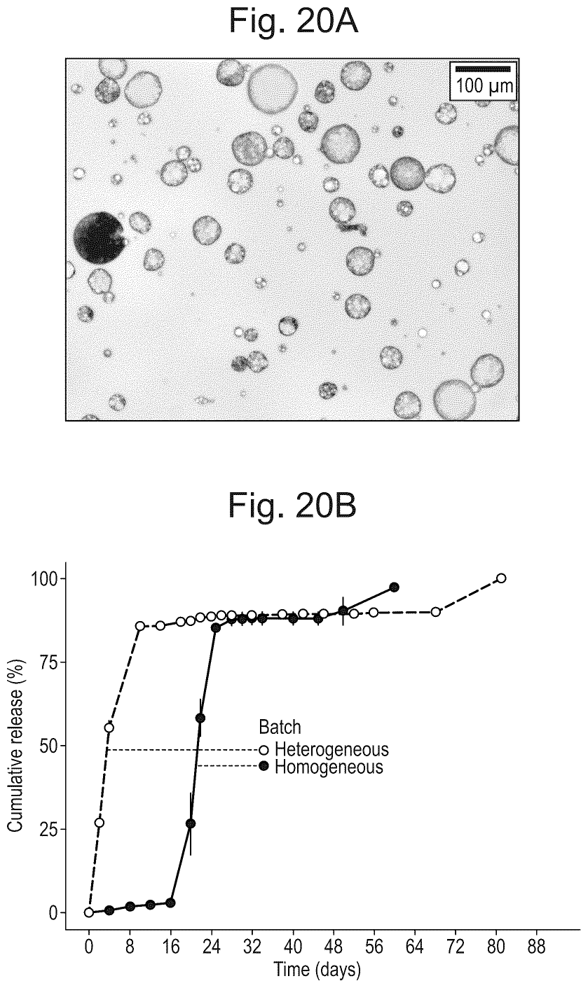

Heterogeneous particles were produced by vortexing using the same flow phase compositions. Firstly, 100 μL of aqueous phase was added to 500u L of solvent polymer phase and vortexed at 3000 rpm for 1 min in a 2 mL LoBind Eppendorf. This mix was added to 10 mL of aqueous extraction phase in a glass vial and vortexed at 2500 rpm for 1 min. Particles were left inside a large volume of aqueous extraction phase for 24 hrs to extract the solvent and harden the particles, resulting in the heterogeneous population shown in FIG. 20A.

The release from each batch was evaluated in vitro following the same protocol described in Example 2, with regular sampling of the fluorescent dextran-TRITC released.

As illustrated in FIG. 20B, heterogeneous particle batches produced by vortexing (FIG. 20B, blue in original colour image) are rapidly releasing the majority of their payload over the first 10 days without any delay compared to homogeneous particles produced by microfluidics (FIG. 20B, red in original colour image). Thus, homogeneous particles are more suited to be used in the delivery of a booster dose in the context of vaccination due to their ability to delay their release.

This experiment demonstrates that whereas current malaria vaccination requires a multiple injections regimen, encapsulation using particles as disclosed herein reduces the need for multiple injections.

### Example 9

The ability of R21 (see Example 7) to elicit immunogenicity after being encapsulated and injected in vivo was assessed by immunising mice with the following regimens:
Prime: a single injection of R21 alone (suspended) in PBS
Particle: a single injection of R21 alone encapsulated in about 50,000 LCSS particles made with 7-17 kDa MW, 50:50 L:G PLGA R502, also referred to herein as Short formulation
Prime+Particle: a single injection of R21 alone in PBS combined with R21 encapsulated in about 50,000 LCSS Short formulation particles.
Prime/Boost: two injections of R21 alone in PBS separated by 3 weeks (titres only measured at week 5, the previously observed antibody peak titre)

Mice receiving the Particle or Prime+Particle regimens were injected with ˜50,000 particles to match the soluble priming dose of R21 (1 μg), each particle containing only a fraction of the total dose.

BALB/c mice (n=8 per group) were immunised with four different regimens by intramuscular injection. Injections were performed using 100 μL Hamilton glass syringes sterilised with ethanol and 25G needles. Injection volume was 50 μL with 0.5% carboxymethylcellulose in PBS as a viscoenhancer agent to prevent LCSS particles aggregation in the syringe. Immunogenicity was monitored by weekly blood sampling followed by IgG quantification by indirect ELISA using a portion of the C-term fragment (pool of peptides covering 67 amino acids, “Cterm”) as the antigen. Comparisons were performed on the peak antibody titre observed from the weekly monitoring for each mouse, and a threshold of 0.5-fold change in antibody titre was used to determine non-inferiority between groups.

R21 Immunogenicity is Preserved when Encapsulated Inside Particles

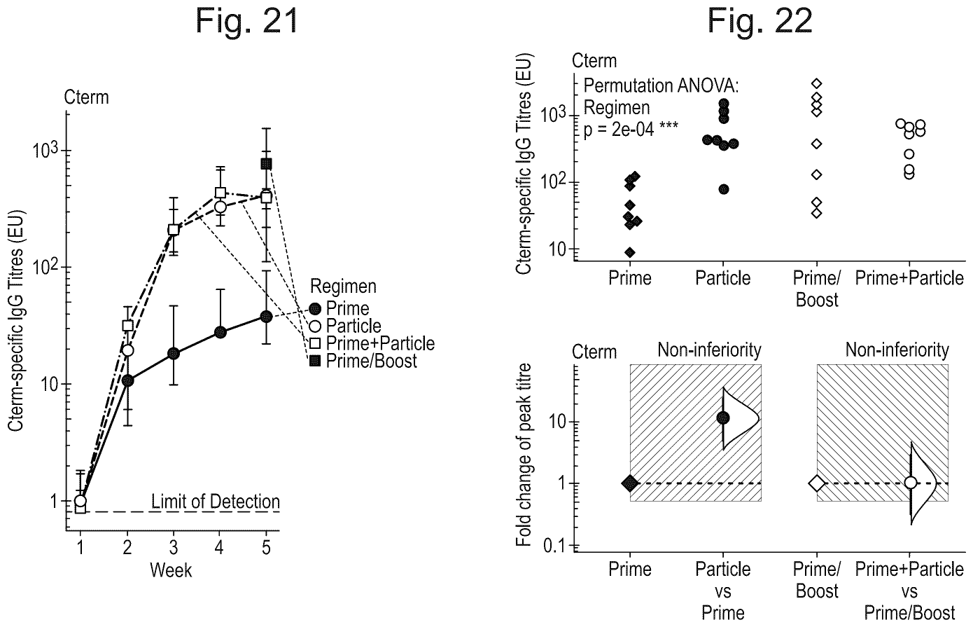

The Particle and Prime+Particle regimens were compared to the Prime and Prime/Boost control regimens. The immune response kinetic profiles are shown in FIG. 21. The profiles did not indicate any qualitative negative impact of the encapsulation process on the immune response. On the contrary, the response to the Cterm fragment appeared to be enhanced by the encapsulation when comparing the Prime regimen to the Particle regimen.

The observed maximum IgG peak for each formulation and the fold change of the mean peak log titres between dose-matched soluble and encapsulated formulations are shown in FIG. 22. Estimated geometric means and confidence intervals for the four regimens are reported in Table 1. Different regimens induced significantly different levels of Cterm specific IgG, as tested by permutation ANOVA (NANP p=0.0016, Cterm p=0.0002). The encapsulation of R21 resulted in 11-fold higher (95% CI 4.8-25, p<0.05) levels of Cterm-specific IgG titres compared to the corresponding prime only control. In contrast, there was no sufficient evidence that the Prime+Particle regimen was non-inferior to the Prime/Boost regimen, with fold changes of 1 (95% CI 0.3-3.3, p>0.05) for the Cterm response.

TABLE 1
Geometric mean estimates of peak antibody
titres for unadjuvanted R21 regimens.

| | Cterm specific IgG titres |
|---|---|
| Regimen | Geometric mean EU [95% CI] |
| Prime | 4.1 · 10¹ |
| | [2.2 · 10¹-7.1 · 10¹] |
| Particle | 4.7 · 10² |
| | [2.3 · 10²-7.9 · 10²] |
| Prime + Particle | 3.9 · 10² |
| | [2.2 · 10²-5.8 · 10²] |
| Prime/Boost | 4 · 10² |
| | [1.2 · 10¹-1.1 · 10³] |

Adjuvant Effect of the Adjuvant is Maintained when Co-Encapsulated Inside Particles

Protein antigens require the addition of adjuvants to increase their immunogenicity. In this study, an adjuvant, a liposome-based adjuvant with saponin QS21 and a TLR-4 agonist integrated into the liposomal bilayer, was used. To assess retention of its adjuvant effect when co-encapsulated with R21 in LCSS particles, immune responses from the above Prime and Particle regimens were compared to immune responses to similar regimens, with the inclusion of the adjuvant. These are designated as Prime-Adj and Particle-Adj, respectively. For the Particle-Adj regimen the same Short formulation was used as for the Particle regimen.

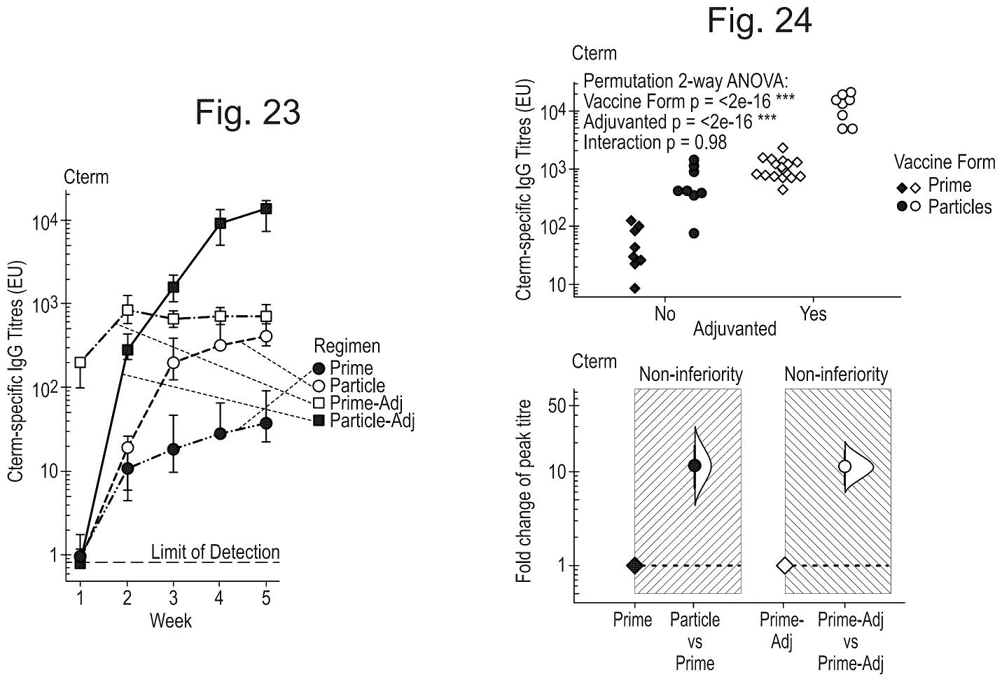

Mice receiving the Particle-Adj regimen were injected with ˜100,000 LCSS particles to match the soluble priming dose of R21 in the Prime-Adj regimen (total of 2 μg). BALB/c mice were immunised with different regimens (Prime-Adj n=16, otherwise n=8) by intramuscular injection. Injections were performed using 100 μL Hamilton glass syringes sterilised with ethanol and 25G needles. Injection volume was 50 μL with 0.5% carboxymethylcellulose in PBS as a viscoenhancer agent to prevent particle aggregation in the syringe. As the adjuvant was supplied in a fixed concentration, and to avoid injection of excessive number of particles that would result in needle blockage, the amount of adjuvant co-encapsulated with R21 was lower than the amount given in the unencapsulated regimen. Thus, mice in the Particle-Adj regimen received 1/7^{th} of the standard adjuvant dose. The resulting immune response kinetic profiles are shown in FIG. 23.

The addition of the adjuvant provided an increase in the immune response against both epitopes in the Prime regimen from day 7 post-immunisation. Co-encapsulation of adjuvant and R21 in LCSS particles resulted in a delay of the immune response against both epitopes compared to the Prime-Adj regimen.

The observed maximum IgG peak for each formulation and the fold change of the mean peak log titres between dose-matched soluble and encapsulated formulations are shown in FIG. 24. Estimated geometric means and confidence intervals for the different regimens are reported in Table 2. The addition of the adjuvant induced significantly different levels of Cterm specific IgG across encapsulated and nonencapsulated formulations, as tested by permutation ANOVA (NANP p<2×10⁻¹⁶, Cterm p<2×10⁻¹⁶). Encapsulation significantly affected the Cterm-specific response (p<2×10⁻¹⁶) across adjuvanted and nonadjuvanted formulations. Interactions between the effect of the encapsulation and the addition of adjuvant were not significant. Encapsulation of R21 with the adjuvant increased immunogenicity compared to nonencapsulated controls, with a 11-fold change (95% CI 7-17, p<0.05) in Cterm-specific IgG titres.

TABLE 2
Geometric mean estimates of peak antibody
titres for the adjuvanted R21 regimens.

| | Cterm specific IgG titres |
|---|---|
| Regimen | Geometric mean EU [95% CI] |
| Prime-Adj | 1 · 10³ |
| | [8.2 · 10²-1.2 · 10³] |
| Particle-Adj | 1.1 · 10⁴ |
| | [7.1 · 10³-1.6 · 10⁴] |

In the subsequent experiments, when co-encapsulated in Particle regimens, the adjuvant was used at 1/7^{th} of the full dose.

### Different Particle Formulations Affect Immunogenicity and Immune Response Delay

In order to investigate the effect of encapsulation on immunogenicity and delay of the immune response with different particle formulations, data from Prime-Adj and Particle-Adj (Short formulation) regimens were combined with that from two additional formulations of particles co-encapsulating R21 and the fractional dose ( 1/7^{th}) of adjuvant. The immune response was monitored by weekly quantification of antibody titres. A summary of the regimens for each mouse and their designation in the figures is provided below:
Short: a single injection of R21 and fractional adjuvant dose encapsulated in LCSS particles made of 7-17 kDa Mw, 50:50 L:G PLGA R502 with in vitro observed delay of 21 days.
Medium: a single injection of R21 and fractional adjuvant dose encapsulated in LCSS particles made of 54-69 kDa Mw, 50:50 L:G PLGA R505 with in vitro observed delay of 33 days.
Long: a single injection of R21 and fractional adjuvant dose encapsulated in LCSS particles made of 24-38 kDa Mw, 75:25 L:G PLGA R753S with in vitro observed delay of 77 days.
Prime: a single injection of R21 and full standard adjuvant dose in PBS.

Mice receiving Particle regimens were injected with ˜100,000 LCSS particles to match the soluble priming dose of R21 (2 μg here). BALB/c mice were immunised with different regimens (Prime n=16, other regimens n=8) by intramuscular injection. Injections were performed using 100 μL Hamilton glass syringes sterilised with ethanol and 25G needles. Injection volume was 50 μL with 0.5% carboxymethylcellulose in PBS as a viscoenhancer agent to prevent particles aggregation in the syringe.

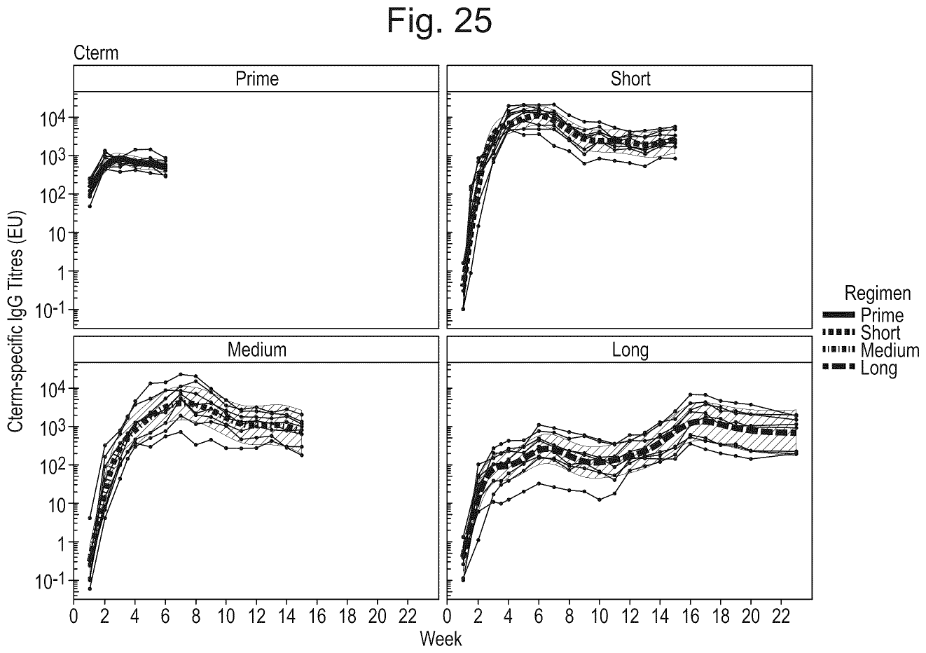

The longitudinal immune response kinetics profiles of the NANP and Cterm-specific responses are shown in FIG. 25 with corresponding generalised additive model (GAM) fits performed on the log-transformed data.

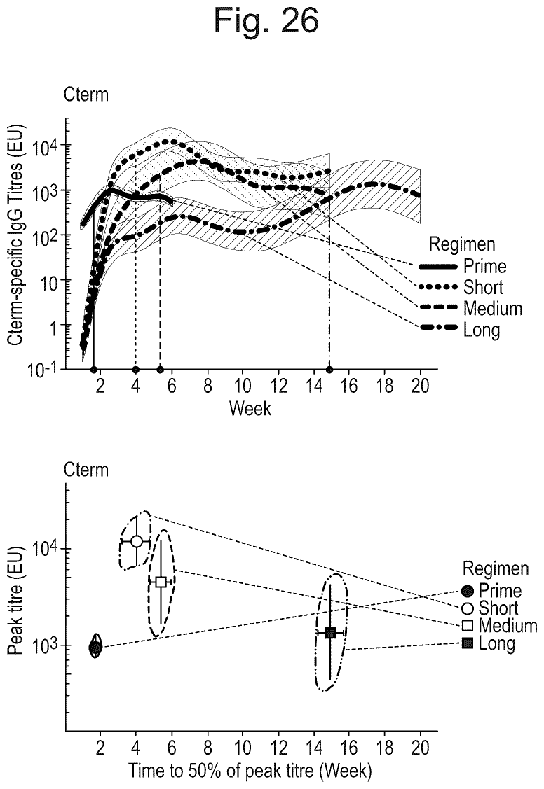

The peak antibody titre and time to 50% of the peak titre, as estimated from the fit, are summarised with their 95% CI in Table 3 and shown in FIG. 26. The time taken to reach 50% of the peak titre (indicated by vertical bars) was chosen as representative of the immune response delay, as 0.5-fold change was decided as the threshold for non-inferiority.

TABLE 3
Estimates of peak antibody titres and time to 50% peak titre
for NANP-specific and Cterm-specific responses for the different
particle formulations and the unencapsulated control.

| | Peak^{a} | Time to 50% Peak^{b} |
|---|---|---|
| Regimen | EU [95% CI] | Weeks [95% CI] |
| Prime | 9.5 · 10² | 1.7 |
| | [7.6 · 10²-1.2 · 10³] | [1.6-1.8] |
| Short | 1.2 · 10⁴ | 4 |
| | [6.8 · 10³-2.1 · 10⁴] | [3.2-4.6] |
| Medium | 4.4 · 10³ | 5.3 |
| | [1.7 · 10³-1.2 · 10⁴] | [4.8-6] |
| Long | 1.7 · 10³ | 15 |
| | [4.4 · 10²-4.2 · 10³] | [14-16] |

^{a}Geometric mean.
^{b}Mean.

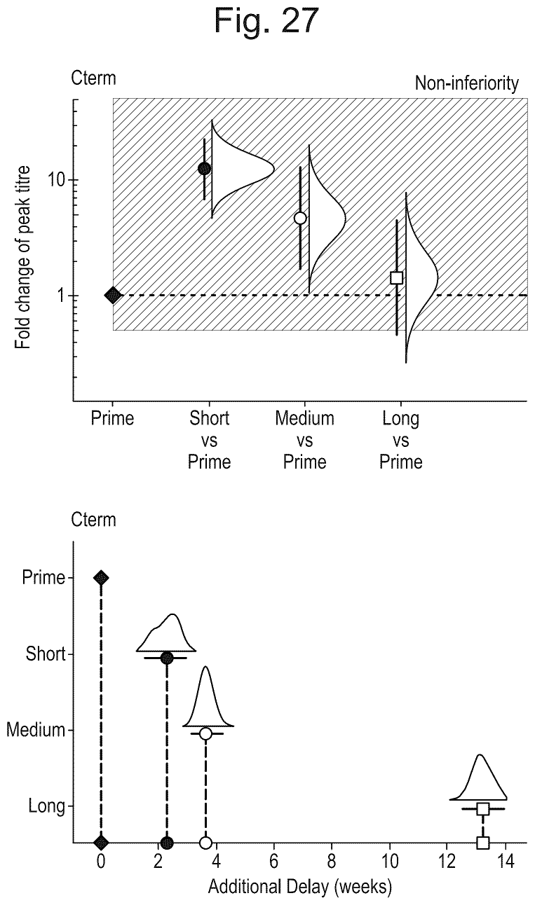

As illustrated in FIG. 27, the effect of encapsulation of R21 with adjuvant was tested for non-inferior immunogenicity (by comparison of the antibody peak titres of the encapsulated regimens to the Prime regimen, with a 0.5-fold threshold for non-inferiority) and a delay in onset of the immune response, indicating lag time in the release of the booster dose. The difference in immune response delay between the different particle formulations was then estimated.

In the Short formulation, encapsulation increased immunogenicity compared to the non-encapsulated control, 12-fold higher (95% CI 6.8-23, p<0.05) Cterm-specific peak antibody titres. The encapsulation in Short particles extended the delay in the onset of immunogenicity compared to the nonencapsulated control, increasing the time to 50% of the peak titre 2.3 weeks (95% CI 1.5-3, p<0.05) for the Cterm-specific antibody titres (FIG. 27). In the Medium formulation, encapsulation led to increased Cterm-specific immunogenicity, with 4.6-fold higher (95% CI 1.7-13, p<0.05) Cterm-specific peak antibody titres. The encapsulation in Medium particles extended the delay in the immunogenicity compared to nonencapsulated control, increasing the time to 50% of the peak titre by 3.7 weeks (95% CI 3.1-4.3, p<0.05) for the Cterm-specific antibody titres. Finally, in the Long formulation, the encapsulation in Long particles provided a significant extension of delay in the immunogenicity compared to nonencapsulated control, increasing the time to 50% of the peak titre by 13 weeks (95% CI 13-14, p<0.05) for the Cterm-specific antibody titres.

The particle formulations induced significantly different delays in Cterm-specific immune response compared to each other. Thus, compared to the Short particle formulation, the Medium particle formulation, made with higher Mw PLGA, increased the time to 50% of peak titre by 1.4 weeks (95% CI 0.5-2.3, p<0.05) for the Cterm-specific response. Similarly, the Long particle formulation, manufactured with a higher L:G ratio PLGA increased the time to 50% of peak titre by 9.5 weeks (95% CI 8.7-10, p<0.05) for the Cterm-specific response compared to the Medium particle formulation.

### Prime+Particle Formulation Provides Superior Immunogenicity to Prime Alone

The R21 vaccine in combination with the adjuvant has demonstrated high immunogenicity and protection in malaria challenge studies when given in a multi-injection regimen. To evaluate the potential benefit of co-injected prime and encapsulated doses (designated as the “Prime+PAR” regimen) over the single matched dose injection (designated as “Prime” regimen), both Short and Medium particle formulations were evaluated in parallel studies. For each study, BALB/c mice were injected with 3 different regimens, and immunogenicity and efficacy in a malaria challenge model were compared.

The regimens were as presented below:
Prime: a single injection of R21 and adjuvant in PBS, matching the total amount received in the Prime+PAR regimens (2 full doses of R21, 1+ 1/7^{th} dose of adjuvant),
Prime/Boost: a prime injection of R21 and adjuvant in PBS, and a booster injection of R21 and adjuvant in PBS. Booster was given two weeks after the prime for the Short study, and 3 weeks after prime for the Medium study.
Prime+Short PAR/Prime+Medium PAR: a single injection of R21 and adjuvant in PBS, and R21 with a fractional dose ( 1/7^{th}) of adjuvant co-encapsulated in either Short or Medium formulation LCSS particles,

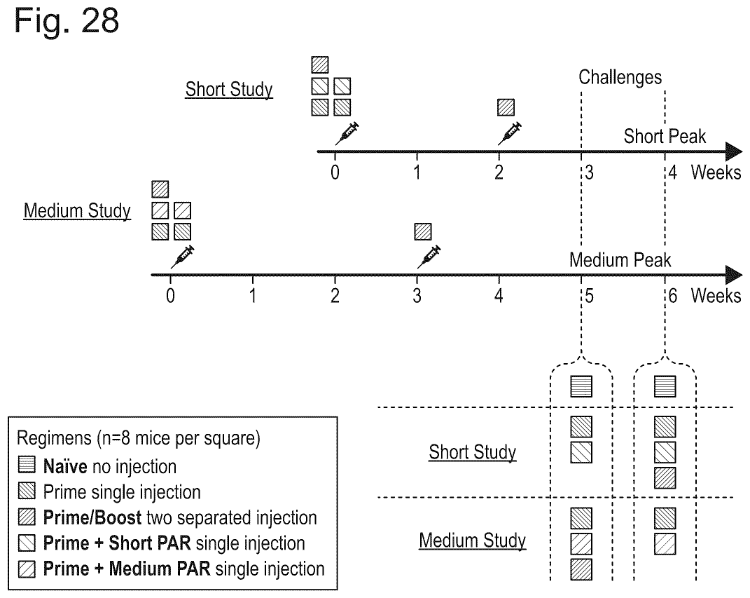

Weekly blood samples were taken to follow the immune response until the malaria challenge session. Experimental design is illustrated in FIG. 28.

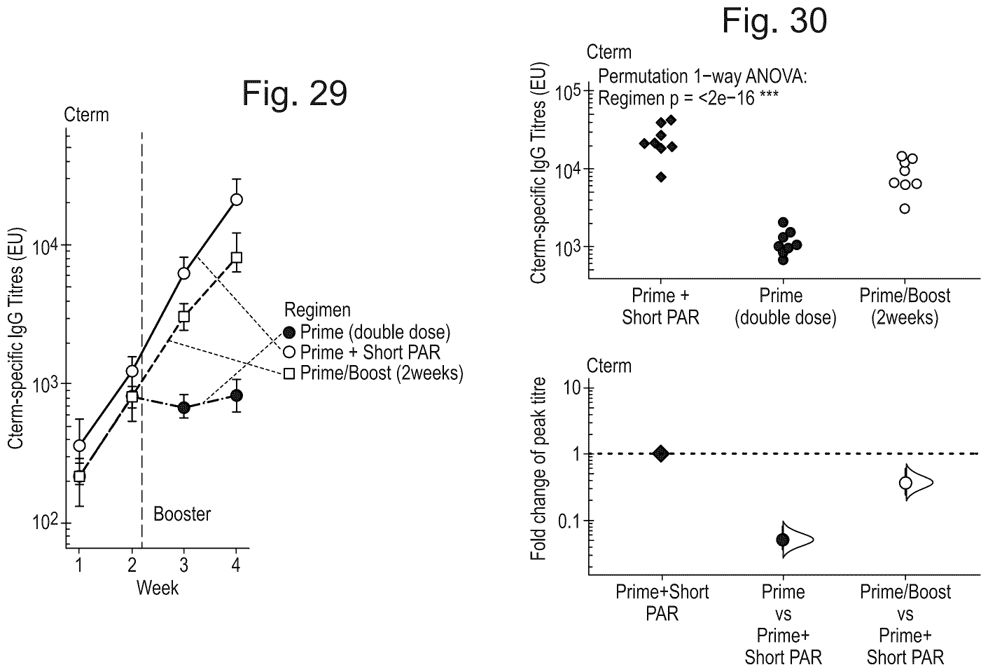

As illustrated in FIG. 29, all three regimens in the Short study had similar Cterm immune response for the first 2 weeks. As expected, the booster dose produced a rise in antibody titres for the Prime/Boost regimen which was similar to the Prime+Short PAR regimen for the Cterm-specific antibody titres. Cterm-specific responses in the Prime regimen reached a plateau after week 3. The observed maximum IgG peak for each regimen and the fold change of the mean peak log titres between encapsulated regimen and nonencapsulated controls are shown in FIG. 30. Estimated geometric means and confidence intervals for the three regimens are reported in Table 4. The Prime+Short PAR resulted in higher Cterm-specific immunogenicity compared to both the Prime, with 20-fold higher (95% CI, 12-28, p<0.05) antibody titres, and the Prime/Boost regimen, with 2.7-fold higher (95% CI, 1.7-4.3, p<0.05) antibody titres.

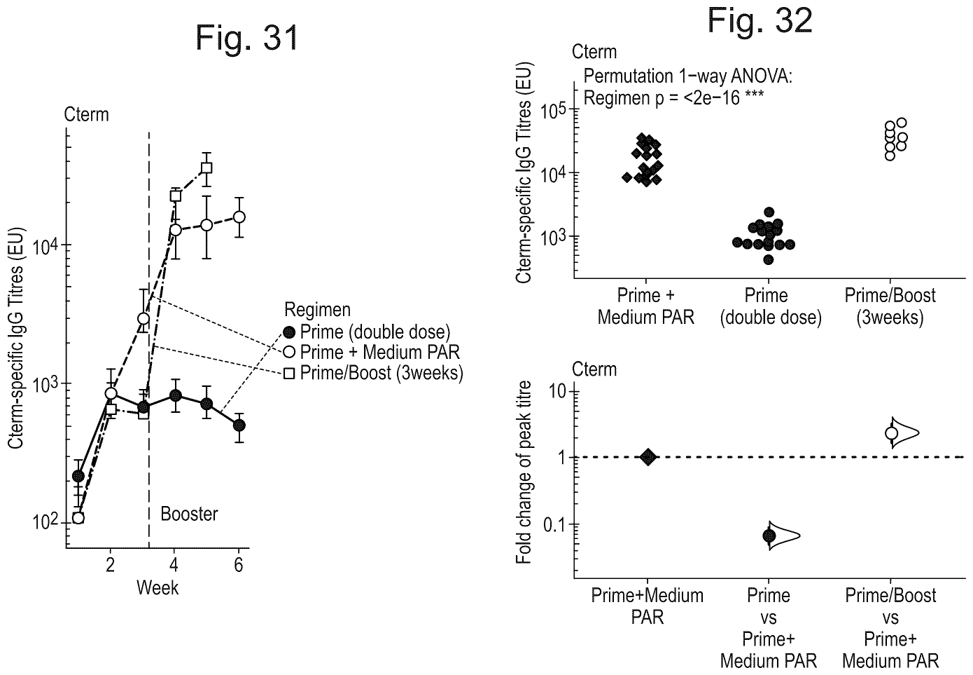

The Prime+Medium PAR regimen generated a stronger immune response than the Prime regimen against the Cterm epitopes (FIG. 31). The kinetics however diverged after week 2, with titres in the Prime+Medium PAR group continuing to rise while those in the Prime regimen reached a plateau. The plateau eventually reached by the Prime+Medium PAR regimen was between those for the Prime and Prime/Boost groups. The observed maximum IgG peak for each regimen and the fold change of the mean peak log titres between encapsulated regimen and nonencapsulated controls are reported in FIG. 32. Estimated geometric means and confidence intervals for the three regimens are reported in Table 4. The Prime+Medium PAR induced higher immunogenicity against Cterm epitopes compared to Prime only, with 2.6-fold higher (95% CI, 1.6-4.2, p<0.05) NANP-specific, and 15-fold higher (95% CI, 11-21, p<0.05) Cterm-specific antibody titres. In contrast, Cterm-specific immunogenicity was lower in comparison with the Prime/Boost regimen, with a 0.4-fold change (95% CI, 0.3-0.6, p<0.05) in antibody titres.

The estimated maximum observed peak titres for both the Short and Medium studies are summarised in Table 4 below.

TABLE 4
Geometric mean estimates of peak antibody titres
for Short and Medium formulations studies.

| | Short Study | Medium Study |
|---|---|---|
| Regimen | Cterm | Cterm |
| | EU [95% CI] | EU [95% CI] |
| Prime | 1.1 · 10³ | 1 · 10³ |
| | [9.1 · 10²-1.4 · 10³] | [8.3 · 10²-1.2 · 10³] |
| Prime + PAR | 2.2 · 104 | 1.5 · 10⁴ |
| | [1.5 · 10⁴-2.9 · 10⁴] | [1.2 · 10⁴-2 · 10⁴] |
| Prime/Boost | 8 · 10³ | 3.4 · 10⁴ |
| | [5.3 · 10³-1.1 · 10⁴] | [2.6 · 10⁴-4.4 · 10⁴] |

### Encapsulation Improves the Efficacy of a Single Vaccine Dose

For each study, the animals in the Prime and Prime+PAR regimens were split into two groups of 8 mice to be challenged at two timepoints: at week 3 and 4 post immunisation in the Short study, and at week 5 and 6 post immunisation in the Medium study, sharing the challenge sessions between studies. This corresponds to the estimated time intervals covering their respective peak antibody titres following particle release. Prime/Boost controls (n=8 per study) were only challenged once, 2 weeks after the boost. Naïve mice (n=8 per challenge session) were shared between the two studies. The challenge design is summarised in FIG. 28.

Malaria challenge was performed by IV injection of 1000 P. falciparum sporozoites. Development of parasitaemia was monitored from day 5 by assessing the presence and percentage of infected erythrocytes in peripheral blood using thin blood smears. Animals that reached 1% parasitaemia were culled. Animals that remained free from infection at day 12 were considered sterilely protected. The effect of regimen on the time to infection was analysed by Cox regression and hazard ratios were computed, adjusted for the challenge session. The hazard ratio for a regimen of interest represents the instantaneous relative risk of infection at any given time, relative to the Prime regimen.

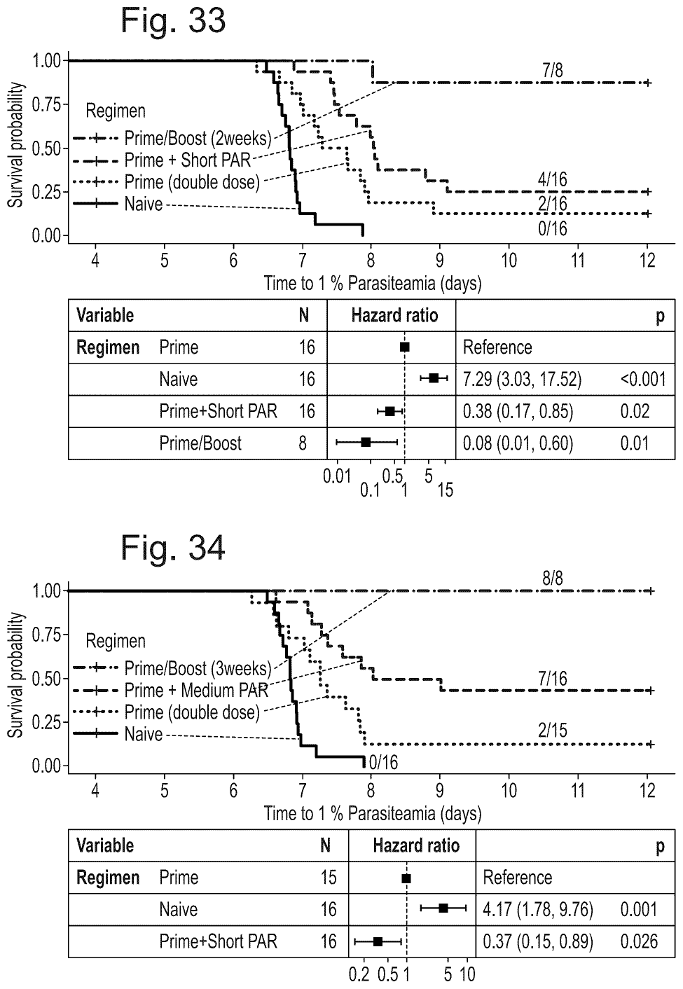

The single injection of the Prime+Short PAR formulation conferred 25% protection (4/16 mice) while in the Prime only group 12.5% (2/16 mice) survived, for the mice in this study. The Prime+Short PAR formulation was more effective at delaying infection compared to the Prime only (FIG. 33), with a hazard ratio of 0.4 (95% CI 0.2-0.9, p=0.02). The Prime/Boost regimen offered 87.5% protection (7/8 mice) and was also more effective than the Prime only, with a hazard ratio of 0.08 (95% CI 0.01-0.6, p=0.01).

The single injection of the Prime+Medium PAR formulation conferred 43.8% (7/16 mice) sterile protection against malaria in comparison with 13.3% (2/15 mice) for the Prime only, observed for the mice in this study. The Prime+Medium PAR formulation was also significantly more effective at delaying infection compared to Prime only (FIG. 34), with a hazard ratio of 0.4 (95% CI 0.2-0.9, p=0.026). The Prime/Boost regimen resulted in 100% sterile protection (8/8 mice). In the absence of infection in the Prime/Boost regimen, the hazard ratio could not be estimated.

### DISCUSSION

### R21 Remains Immunogenic Following Encapsulation

Short delay release formulation LCSS particles encapsulating the R21 vaccine resulted in non-inferior Cterm responses compared to the same dose of unencapsulated vaccine. This suggests that the microfluidic emulsification process and exposure to the physiological conditions in vivo following the injection of encapsulated R21 did not affect immunogenicity for either of the studied epitopes, demonstrating the utility of this technology with the malaria vaccine. Moreover, the response against the Cterm epitope was significantly higher in the Particle regimen compared to Prime, indicating that the kinetics of release and resulting delayed exposure to the booster vaccine dose increased the magnitude of the Cterm-specific immune response. The low immunogenicity of unadjuvanted R21 hindered the comparison of delays in generating immunogenicity and relevant peak titre differences, as all previous preclinical work with R21 includes use of adjuvants.

Using the adjuvant, co-encapsulation of R21 and a fractional dose of adjuvant in Short delay release formulation particles demonstrated the same ability to maintain or improve immunogenicity compared to unencapsulated adjuvanted vaccine. Cterm-specific response was significantly increased by more than 12-fold, despite the 7-fold lower adjuvant dose.

### Delay in the Cterm-Specific Response can be Tuned by the LCSS Particle Formulation

Overall, the immunogenicity results suggest that the Cterm-specific response increases with repeated exposure to the R21 vaccine, as reported previously. Indeed, mice that only received a single Prime injection had the lowest Cterm-specific antibody titres, while mice that also received a booster, demonstrated a significant increase in Cterm-specific immunogenicity (FIG. 22). In the case of LCSS particles, as observed in vitro, the kinetic of delivery of the encapsulated vaccine payload is the result of the cumulative release of each single particle. The individual releases happen after a lag slightly different for each particle resulting in a global release from all particles that can span from days to weeks depending on the LCSS particle formulation. Thus, multiple releases from different fractions of the total injected particles would result in multiple exposures to the vaccine in vivo, producing higher levels of Cterm-specific antibody response than the single exposure at a discrete time, as in Prime only regimens. Cterm immunogenicity monitoring can thus be used to detect delays in the vaccine release, with the timing of the antibody peak titre indicating the end of exposure to the vaccine. Short, Medium and Long formulations of LCSS particles demonstrated the ability to induce significant delays in Cterm-specific peak response compared to a Prime only regimen, ranging from 2 additional weeks for the Short formulation, to 13 weeks for the Long formulation. The delay was consistent with the order of release observed in vitro (Short delay <Medium delay <Long delay) and expected from the PLGA composition of each formulation (increased Mw for Medium formulation, increased L:G for Long formulation), demonstrating the tuneability of this technology.

### The Addition of Encapsulated Booster to the Prime Regimen Increases Cterm-Specific Immune Responses

Different immunogenic patterns were observed when co-injecting a standard priming vaccine dose with Short or Medium particle formulations containing the booster vaccine with a fractional ( 1/7^{th}) dose of the adjuvant. The Prime+Medium PAR regimen induced 15-fold higher Cterm-specific antibody titres compared to Prime only matched dose regimens (FIG. 32). The Prime+Short PAR regimen induced only 20-fold higher Cterm-specific Ab titres than the Prime only (FIG. 30).

### Protection Against Malaria was Increased by the Addition of the Encapsulated Booster Dose to a Soluble Prime

Regardless of their different immunogenicity profiles, the two Prime+Particle (Short and Medium) regimens provided a significant hazard ratio reduction in the malaria challenge experiments compared to Prime only, showing that booster dose encapsulation can enhance protection against malaria in mice by more than 2.5-fold when administered as a single shot. The observed sterile protection of co-injected Prime and particle regimens was higher than Prime only, highlighting the additional benefit in efficacy provided by the particles in single injections. When Medium formulation particles were combined with a priming dose, 44% of the mice survived, a 2-fold higher protection than the Short formulation particles (25%) and a 3.3-fold higher protection than the Prime only (13%).

These results are the first demonstration of the benefit of an injectable delayed delivery system in a disease challenge model, with a clinically relevant vaccine, in the context of single-dose vaccine development.

### Definitions

It should be appreciated that “embodiments” of the disclosure can be specifically combined together unless the context indicates otherwise. The specific combinations of all disclosed embodiments (unless implied otherwise by the context) are further disclosed embodiments of the claimed invention.

All publications, patents and patent applications cited herein, whether supra or infra, are hereby incorporated by reference in their entirety.

Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art in connection with biological substances such as polynucleotide and polypeptides. Practitioners are particularly directed to “March's Advanced Organic Chemistry”. Wiley, 2020 for definitions and terms of art in connection with chemical substances, including synthetic reactions for their synthesis.

The skilled practitioner is also directed to Remington's Pharmaceutical Sciences, 20th Edition, 2000, pub. Lippincott, Williams & Wilkins for further information regarding techniques and protocols mentioned herein.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meichofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. A peptide can be made using recombinant techniques, e.g., through the expression of a recombinant or synthetic polynucleotide. A recombinantly produced peptide is typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypepties that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

The term “alkyl” refers to a linear or branched hydrocarbyl group of formula (CₙH₂ₙ₊₁). Examples include methyl, ethyl, n-propyl, iso-propyl, n-butyl, iso-butyl, sec-butyl, and tert-butyl.

The term “aryl” refers to a substituted or unsubstituted, monocyclic or fused polycyclic aromatic group typically containing from 6 to 10 carbon atoms in the ring portion. Examples include monocyclic groups such as phenyl and fused bicyclic groups such as naphthyl and indenyl. Phenyl(benzene) is typically preferred.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Hydrochloride salts and acetate salts are preferred, in particular hydrochloride salts.

Unless otherwise required by the context, the stercochemistry of compounds disclosed herein is not limited. In particular, the particles disclosed herein may comprise components containing one or more chiral centre. Such components may be used in enantiomerically or diastereoisomerically pure form, or in the form of a mixture of isomers. Further, for the avoidance of doubt, such components may be used in any tautomeric form. Typically, an active agent or substance described herein is at least 50%, preferably at least 60, 75%, 90% or 95% enantiomerically or diasteriomerically pure. Typically, a compound disclosed herein comprises by weight at least 60%, such as at least 75%, 90%, or 95% of a single enantiomer or diastereomer. Preferably, a disclosed compound is substantially optically pure.

1. A method of preparing particles comprising an aqueous core encapsulated by a polymer shell, the method using a flow chip having channels formed therein including a main channel, the method comprising:
flowing a solvent phase having droplets of the aqueous phase entrained therein through a transfer section of the main channel, wherein the aqueous phase comprises a pharmaceutical agent and an aqueous solvent, and the solvent phase comprises a polymer and a non-aqueous solvent;
flowing an extraction phase through extraction phase inlet channels in the flow chip, the extraction phase comprising an extraction solvent, wherein the extraction phase inlet channels are provided on opposite sides of the main channel, and the transfer section and the extraction phase inlet channels open into an extraction phase intersection section of the main channel, so that droplets of the solvent phase, which encapsulate droplets of the aqueous phase, are formed in the extraction phase; and
flowing the extraction phase having the droplets of the solvent phase entrained therein through an extraction section of the main channel extending from the extraction phase intersection section; and
extracting the non-aqueous solvent of the aqueous phase, so that the particles are formed with the aqueous core being formed by the droplets of the aqueous phase and the shell being formed by the polymer,
wherein the main channel has an increase in height at a location where the transfer section opens into the extraction phase intersection section or downstream thereof.

2. A method according to claim 1, wherein the main channel has an increase in height at a location where the transfer section opens into the extraction phase intersection section.

3. A method according to claim 1, wherein the main channel has an increase in height downstream of the location where the transfer section opens into the extraction phase intersection section.

4. A method according to any one of the preceding claims, wherein the main channel has a height, before the increase in height, in a range from 5 μm to 250 μm.

5. A method according to any one of the preceding claims, wherein the main channel has a height, after the increase in height, in a range from 20 μm to 500 μm.

6. A method according to any one of the preceding claims, wherein the increase in height of the main channel is at least 15 μm.

7. A method according to any one of the preceding claims, wherein the extraction phase inlet channels have a width in a range from 20 μm to 500 μm.

8. A method according to any one of the preceding claims, wherein the extraction section comprises an extraction nozzle section downstream of the extraction phase intersection section, the extraction nozzle section having an increase in width with distance from the extraction phase intersection section.

9. A method according to claim 8, wherein the extraction nozzle section comprises a neck section and an expansion section downstream of the neck section, the increase in width of the extraction nozzle section occurring in the expansion section.

10. A method according to claim 9, wherein:
the neck section of the extraction nozzle section has a width in a range from 10 μm to 200 μm;
the neck section of the extraction nozzle section has a length in a range from 10 μm to 200 μm;
the expansion section of the extraction nozzle section has a maximum width in a range from 100 μm to 1000 μm; and/or
the expansion section of the extraction nozzle section has a length in a range from 10 μm to 1000 μm.

11. A method according to any one of the preceding claims, wherein the flow rate of the extraction phase through the extraction phase inlet channels is greater than the flow rate of the solvent phase through the transfer section.

12. A method according to any one of the preceding claims, wherein the flow rate of the solvent phase through the transfer section is in a range from 2.5 nL/s to 150 μL/s.

13. A method according to any one of the preceding claims, wherein the flow rate of the extraction phase through the extraction phase inlet channels is in a range from 12 nL/s to 3 mL/s.

14. A method according to any one of the preceding claims, further comprising:
flowing the aqueous phase through an aqueous phase inlet section of the main channel; and
flowing a solvent phase through solvent phase inlet channels in the flow chip, wherein the solvent phase inlet channels are provided on opposite sides of the main channel, and the aqueous phase inlet section and the solvent phase inlet channels open into an solvent phase intersection section of the main channel, so that the droplets of the aqueous phase are formed in the solvent phase,
the transfer section extending from the solvent phase intersection section.

15. A method according to claim 14, wherein the transfer section comprises a solvent nozzle section downstream of the solvent phase intersection section, the solvent nozzle section having an increase in width with distance from the solvent phase intersection section.

16. A method according to claim 15, wherein the solvent nozzle section comprises a neck section and an expansion section downstream of the neck section, the increase in width occurring in the expansion section.

17. A method according to claim 16, wherein:
the neck section of the solvent nozzle section has a width in a range from 5 μm to 200 μm;
the neck section of the solvent nozzle section has a length in a range from 10 μm to 200 μm;
the expansion section of the solvent nozzle section has a maximum width in a range from 100 μm to 500 μm; and/or
the expansion section of the solvent nozzle section has a length in a range from 10 μm to 500 μm.

18. A method according to any one of claims 14 to 17, wherein the flow rate of solvent phase through solvent phase inlet channels is greater than the flow rate of aqueous phase through an aqueous phase inlet section.

19. A method according to any one of claims 14 to 18, wherein the flow rate of aqueous phase through an aqueous phase inlet section is in a range from 0.25 nL/s to 30 μL/s.

20. A method according to any one of the preceding claims, wherein surfaces of main channel of the flow chip upstream of the extraction phase intersection section are hydrophobic.

21. A method according to any one of the preceding claims, wherein
the extraction solvent is hydrophilic,
the surfaces of the extraction phase inlet channels are hydrophilic, and
the surfaces of main channel of the flow chip in the extraction phase intersection section and downstream thereof are hydrophilic.

22. A method according to any one of the preceding claims, wherein the channels have planar extent.

23. A method according to any one of the preceding claims, wherein the flow chip is formed by multilayer moulding.

24. A method according to any one of the preceding claims, wherein the flow chip comprises a body comprising or consisting of PDMS (polydimethylsiloxane), PMMA (polymethylmethacrylate), polycarbonate (PC), cyclic olefin copolymer (COC); or glass.

25. A method according to any one of the preceding claims, wherein the pharmaceutical agent is selected from immunogenic agents, analgesics, antibiotics, anti-thrombotic drugs, antidepressants, anticancer drugs, antiepileptics, anti-inflammatory drugs, antipsychotic agents, antivirals, sedatives, steroids, antidiabetics, cardiovascular drugs, and drugs for pain management, treatment of skin conditions and treatment of brain diseases;
preferably wherein said pharmaceutical agent is a vaccine agent.

26. A method according to any one of the preceding claims, wherein the aqueous solvent comprises an aqueous solution of from about pH 4 to about pH 10;
optionally wherein said aqueous solvent comprises one or more buffer salts and/or one or more gelling agents and/or one or more stabilizers.

27. A method according to any one of the preceding claims, wherein the polymer comprises or consists of one or more biodegradable polymers, wherein said one or more biodegradable polymers are selected from aliphatic polyesters, aromatic copolyesters, polyurethanes, polycarbonates, polyamides, poly(ester-amide) s, polyanhydrides, polysaccharides, and blends thereof or copolymers thereof, and
wherein preferably said one or more biodegradable polymers are selected from poly(lactic-co-glycolic acid) (PLGA), polylactic acid (PLA), polyglycolic acid (PGA), polycaprolactone (PCL), poly(butylene succinate), poly(p-dioxanone) (PPDO), poly(hydroxybutyrate) (PHB), poly(butylene adipate-co-terephtalate) (PBAT)), chitosan, cellulose, hyaluronic acid, and blends thereof and copolymers thereof.

28. A method according to any one of the preceding claims, wherein the non-aqueous solvent is selected from dimethyl carbonate (DMC), dichloromethane (DCM), toluene, chloroform, n-hexane, diethyl ether, benzene, n-butanol, butyl acetate, carbon tetrachloride, cyclohexane, 1,2-dichloroethane, ethyl acetate, heptane, methyl-t-butyl ether, methyl ethyl ketone, pentane, and dicholoroethylene, and mixtures thereof.

29. A method according to any one of the preceding claims, wherein the extraction solvent comprises an aqueous solution of from about pH 4 to about pH 10;
optionally wherein said extraction solvent comprises one or more buffer salts; one or more surfactants; one or more viscoenhancers and/or one or more osmolarity regulators.

30. A method according to any one of the preceding claims, wherein the non-aqueous solvent is extracted by evaporation or by liquid phase extraction into the aqueous solvent.

31. A method according to any one of the preceding claims, wherein the aqueous solvent and the non-aqueous solvent are immiscible.

32. A flow chip for preparing particles comprising an aqueous core encapsulated by a polymer shell, the flow chip having channels formed therein which comprise a main channel and extraction phase inlet channels provided on opposite sides of the main channel,
wherein the main channel comprises:
a transfer section;
an extraction phase intersection section, into which the transfer section and the extraction phase inlet channels open; and
an extraction section extending from the extraction phase intersection section,
wherein the main channel has an increase in height at a location where the transfer section opens into the extraction phase intersection section or downstream thereof.

33. A device according to claim 32, wherein
the channels further comprise solvent phase inlet channels on opposite sides of the main channel, and
the main channel further comprises:
an aqueous phase inlet section; and
an solvent phase intersection section, into which the aqueous phase inlet section and the solvent phase inlet channels open,
the transfer section extending from the solvent phase intersection section.

34. An aqueous-core polymeric-shell particle, comprising:
an aqueous core comprising a pharmaceutical agent dissolved or dispersed in an aqueous solvent; and
a homogeneous solid biodegradable polymeric shell encapsulating the aqueous core.

35. A particle according to claim 34, wherein:
the pharmaceutical agent is as defined in claim 25; and/or
the aqueous solvent is as defined in claim 26; and/or
the polymeric shell comprises or consists of one or more polymers as defined in claim 27.

36. A particle according to claim 34 or 35, wherein the diameter of said particle is from about 5 μm to about 500 μm; and/or wherein the polymeric shell has a thickness of from about 0.1 μm to about 100 μm;
wherein preferably the diameter of said particle is from about 20 μm to about 150 μm; and/or the polymeric shell has a thickness of from about 1 μm to about 20 μm.

37. A population of particles according to any one of claims 34 to 36, wherein at least 90% of the particles in the population are characterised as comprising:
a single, spherical, aqueous core volume having a mean diameter of from about 1 μm to about 300 μm and wherein the smallest diameter of the aqueous core is at least 70% of the largest diameter of the aqueous core; and
a biodegradable polymeric shell having a thickness of from about 0.1 μm to about 100 μm and wherein the thickness of the thinnest part of the polymeric shell is at least 70% of the thickness of the thickest part of the polymeric shell.

38. A pharmaceutical composition comprising a plurality of particles according to any one of claims 34 to 36 or a population of particles according to claim 37 and one or more pharmaceutically acceptable excipient, diluent, or adjuvant.

39. A pharmaceutical composition according to claim 38, wherein said composition comprises a further pharmaceutical agent.

40. A prime/boost vaccine composition comprising:
(i) a plurality of particles according to any one of claims 34 to 36 or a population of particles according to claim 37, wherein said particles each comprise a pharmaceutical agent which is a first immunogenic agent; and
(ii) a second immunogenic agent;
wherein the first immunogenic agent and the second immunogenic agent are the same or different.

41. A particle according to any one of claims 34 to 36, a population of particles according to claim 37 or a composition according to any one of claims 38 to 4 for use in in medicine.

42. A composition comprising a plurality of particles according to any one of claims 34 to 36 and optionally further comprising one or more further therapeutic agents for use in a method of vaccination, preferably prime/boost vaccination.
